(19) United States
(12) Patent Application Publication
(10) Pub. No.: US 2022/0152150 A1
(43) Pub. Date: May 19, 2022

(54) MESOPOROUS SILICA PARTICLES COMPOSITIONS FOR VIRAL DELIVERY

(71) Applicant: Novartis AG, Basel (CH)

(72) Inventors: Sandeep Tharian Koshy, Cambridge, MA (US); Stephen M. Canham, Cambridge, MA (US)

(21) Appl. No.: 17/433,812

(22) PCT Filed: Feb. 24, 2020
(86) PCT No.: PCT/US2020/019461

§ 371 Date: Aug. 25, 2021

Related U.S. Application Data
(60) Provisional application No. 62/810,260, filed on Feb. 25, 2019.

(51) Int. Cl. A61K 38/17; A61K 47/69; A61K 47/59; A61K 48/00; A61K 47/64; A61K 31/4745; C12N 5/0783; C12N 15/86

(52) U.S. Cl. CPC A61K 38/1774; A61K 47/6923; A61K 47/6901; A61K 47/59; A61K 48/0066; A61K 47/642; C12N 2501/2302; C12N 5/0636; C12N 15/86; C12N 2740/15043; C12N 2501/515; C12N 2501/51; C12N 2501/599; A61K 31/4745

(57) ABSTRACT

The present invention relates generally to the use of compositions including mesoporous silica particles that may be surface modified, for the delivery of viral vectors. In some embodiments, the viral vectors are used to transduce T cells to express a chimeric antigen receptor (CAR), to treat a subject having a disease, e.g., a disease associated with expression of a tumor antigen.

### RELATED APPLICATION

This application claims priority to U.S. Ser. No. 62/810,260 filed Feb. 25, 2019, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Feb. 20, 2020, is named N2067-7161WO_SL.txt and is 232,920 bytes in size.

### FIELD OF THE INVENTION

The present invention relates generally to the use of mesoporous silica compositions for delivery of viral vectors or a drug substance. In some embodiments, the viral vectors include a nucleotide sequence that is engineered to express a chimeric antigen receptor (CAR), to treat a subject having a disease, e.g., a disease associated with expression of a tumor antigen.

### BACKGROUND OF THE INVENTION

T cell adoptive transfer protocols show potential in a number of therapeutic applications, such as cancer, where CAR T cell therapies have recently been approved for the treatment of B cell malignancies. There is a need to deliver virus vectors or drug substances in a localized manner, and find efficient manufacturing processes.

### SUMMARY OF THE INVENTION

Contemplated herein is a composition, comprising a first population of mesoporous silica particles and a viral vector. In some embodiments, the viral vector is conjugated to first population of the mesoporous silica particles. In some embodiments, the viral vector is electrostatically or covalently conjugated to the first population of mesoporous silica particles. In some embodiments, the first population of mesoporous silica particles are surface modified. In some embodiments, the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH2-CH₂—)₁₋₂₅ linker. In some embodiments, the surface modification on the first population of mesoporous silica particles is a primary, secondary, tertiary, or quarternary amine. In some embodiments, the surface modification on the first population of mesoporous silica particles is a polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, the viral vector is a retrovirus, adenovirus, adeno-associated virus, herpes virus, or lentivirus. In some embodiments, the viral vector comprises an expression vector comprising a recombinant polynucleotide comprising an expression control sequence operatively linked to a nucleotide sequence to be expressed. In some embodiments, the nucleotide sequence encodes a chimeric antigen receptor (CAR), an engineered TCR, one or more cytokines, one or more chemokines, an shRNA to block an inhibitory molecule, or wherein the nucleotide sequence comprises an mRNA to induce expression of a protein. In some embodiments, the nucleotide sequence encodes a polypeptide engineered to target a tumor antigen. In some embodiments, the polypeptide targets a tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TESL LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof. In some embodiments, the protein is a CAR that comprises an antigen binding domain, a transmembrane domain, a costimulatory signaling region, and a signaling domain. In some embodiments, the signaling domain is a CD3 zeta signaling domain. In some embodiments, the composition further comprises a T cell stimulating compound or tumor antigen. In some embodiments, the T cell stimulating compound or the tumor antigen is conjugated to or adsorbed on the first population of mesoporous silica particles or a second population of mesoporous silica particles, and wherein the T-cell stimulating compound is IL-2, IL-15, anti-CD2 mAb, anti-CD3 mAb, anti-CD28 mAb, neo-antigen peptides peptides from shared antigens such as TRP2, gp100, tumor cell lysate, CD19, CD20, CD22, ROR1, mesothelin, CD33/IL3Ra, c-Met, PSMA, Glycolipid F77, EGFRvIII, GD-2, NY-ESO-1 TCR, MAGE A3 TCR, or combinations thereof. In some embodiments, the T cell stimulating compound or tumor antigen is conjugated to or adsorbed on the first population of mesoporous silica particles. In some embodiments in which the composition comprises the second population of mesoporous silica particles, the T cell stimulating compound or tumor antigen is conjugated to the second population of mesoporous silica particles or to a lipid envelope on the surface of the second population of mesoporous silica particles. In some embodiments, the composition further comprises a cytokine. In some embodiments, the cytokine is conjugated to or adsorbed on the first or second population of mesoporous silica particles. In some embodiments, the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof. In some embodiments, mesoporous silica particles comprise pores of between 2-50 nm in diameter. In some embodiments, the mesoporous silica particles have a surface area of at least about 100 m²/g. In some embodiments, the composition is suitable for injectable use. In some embodiments, the mesoporous silica particles are in the form of mesoporous silica rods.

Also contemplated herein is a method comprising: contacting T lymphocytes with a composition comprising a first population of mesoporous silica particles and a viral vector; wherein the viral vector comprises an expression vector comprising a recombinant polynucleotide comprising an expression control sequence operatively linked to a nucleotide sequence to be expressed. In some embodiments, the contacting occurs in vitro. In some embodiments, the T lymphocytes are activated before or after contacting with the first population of mesoporous silica particles. In some embodiments, the viral vector is conjugated to the first population of mesoporous silica particles. In some embodiments, the viral vector is electrostatically or covalently conjugated to the first population of mesoporous silica particles. In some embodiments, the first population of mesoporous silica particles are surface modified. In some embodiments, the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH₂—CH₂—)₁₋₂₅ linker. In some embodiments, the surface modification on the first population of mesoporous silica particles is a primary, secondary, tertiary, or quarternary amine. In some embodiments, the first population of mesoporous silica particles are surface modified with polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, the viral vector is a lentivirus, retrovirus, or adenovirus. In some embodiments, the nucleotide sequence encodes a chimeric antigen receptor (CAR). In some embodiments, the CAR is engineered to target a tumor antigen. In some embodiments, the T lymphocytes are activated by contacting the T lymphocytes with a T cell stimulating compound or tumor antigen. In some embodiments, the T cell stimulating compound or tumor antigen is conjugated to or adsorbed on the first population of mesoporous silica particles or a second population of mesoporous silica particles. In some embodiments, the T cell stimulating compound or tumor antigen is conjugated to or adsorbed on the first population of mesoporous silica particles. In some embodiments, the T cell stimulating compound or tumor antigen is conjugated directly to the second population of mesoporous silica particles or to a lipid envelope on the surface of the second population of mesoporous silica particles. In some embodiments, the method further comprises contacting the T lymphocytes with a cytokine. In some embodiments, the cytokine is in the medium or conjugated to or adsorbed on the first or second population of mesoporous silica particles. In some embodiments, the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof. In some embodiments, the mesoporous silica particles are in the form of mesoporous silica rods.

Also contemplated herein is a method of genetically transducing T lymphocytes with a recombinant polynucleotide in vivo, comprising: administering to a subject, having one or more T lymphocytes, a composition comprising a first population of mesoporous silica particles and a viral vector; wherein the viral vector comprises an expression vector comprising a recombinant polynucleotide comprising an expression control sequence operatively linked to a nucleotide sequence to be expressed, and wherein when the composition contacts one or more T lymphocytes, the T lymphocytes are genetically transduced with the recombinant polynucleotide. In some embodiments, the viral vector is conjugated to the first population of mesoporous silica particles. In some embodiments, the viral vector is electrostatically or covalently conjugated to the first population of mesoporous silica particles. In some embodiments, the first population of mesoporous silica particles are surface modified. In some embodiments, the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH₂—CH₂—)₁₋₂₅ linker. In some embodiments, the surface modification on the first population of mesoporous silica particles is a primary, secondary, tertiary, or quarternary amine. In some embodiments, the first population of mesoporous silica particles are surface modified with polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, the viral vector is a lentivirus, retrovirus, or adenovirus. In some embodiments, the nucleotide sequence encodes a chimeric antigen receptor (CAR). In some embodiments, the CAR is engineered to target a tumor antigen. In some embodiments, the composition further comprises a T cell stimulating compound or tumor antigen conjugated to or adsorbed on the first population of mesoporous silica particles or a second population of mesoporous silica particles. In some embodiments, the T cell stimulating compound or tumor antigen is conjugated to or adsorbed on the first population of mesoporous silica particles. In some embodiments, the composition comprises the second population of mesoporous silica particles, and wherein the T cell stimulating compound or tumor antigen is conjugated directly to the second population of mesoporous silica particles or to a lipid envelope on the surface of the second population of mesoporous silica particles. In some embodiments, the first or second population of mesoporous silica particles further comprises a cytokine conjugated to or adsorbed on the first or second population of mesoporous silica particles. In some embodiments, the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof. In some embodiments, the subject's T lymphocytes expand in vivo. In some embodiments, the mesoporous silica particles are in the form of mesoporous silica rods.

Also contemplated herein is a method of expanding a T lymphocyte population in vitro, comprising (a) contacting the T lymphocyte population with a composition comprising a first population of mesoporous silica particles and a viral vector to provide a transduced T lymphocyte population; and (b) contacting the transduced T lymphocyte population with a T cell stimulating compound or tumor antigen and optionally, a cytokine; wherein the viral vector comprises an expression vector comprising a recombinant polynucleotide comprising an expression control sequence operatively linked to a nucleotide sequence to be expressed. In some embodiments, the viral vector is conjugated to the first population of mesoporous silica particles. In some embodiments, the viral vector is electrostatically or covalently conjugated to the first population of mesoporous silica particles. In some embodiments, the first population of mesoporous silica particles are surface modified. In some embodiments, the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH₂—CH₂—)₁₋₂₅ linker. In some embodiments, the surface modification on the first population of mesoporous silica particles is a primary, secondary, tertiary, or quarternary amine. In some embodiments, the first population of mesoporous silica particles are surface modified with polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, the viral vector is a lentivirus, retrovirus, or adenovirus. In some embodiments, the nucleotide sequence encodes a chimeric antigen receptor (CAR). In some embodiments, the CAR is engineered to target a tumor antigen. In some embodiments, the T cell stimulating compound or tumor antigen is conjugated to or adsorbed on the first population of mesoporous silica particles or a second population of mesoporous silica particles, and wherein the T-cell stimulating compound or tumor antigen is IL-2, IL-15, anti-CD2 mAb, anti-CD3 mAb, anti-CD28 mAb, neo-antigen peptides, CD19, CD20, CD22, ROR1, mesothelin, CD33/IL3Ra, c-Met, PSMA, Glycolipid F77, EGFRvIII, GD-2, NY-ESO-1 TCR, MAGE A3 TCR, or combinations thereof. In some embodiments, the T cell stimulating compound or tumor antigen is conjugated to or adsorbed on the first population of mesoporous silica particles. In some embodiments comprising the second population of mesoporous silica particles, the T cell stimulating compound or tumor antigen is conjugated to the second population of mesoporous silica particles or to a lipid envelope on the surface of the second population of mesoporous silica particles. In some embodiments, the method further comprises: (c) contacting the T lymphocytes with a cytokine; wherein the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof. In some embodiments, the mesoporous silica particles are in the form of mesoporous silica rods.

Also contemplated herein is a method of treating a subject having a disease, disorder, or condition associated with an elevated expression of a tumor antigen, the method comprising: administering to the subject a composition comprising a first population of mesoporous silica particles and a viral vector, wherein the viral vector comprises a recombinant polynucleotide comprising an expression control sequence operatively linked to a nucleotide sequence that encodes a chimeric antigen receptor (CAR) that is engineered to target the tumor antigen, thereby treating the subject. In some embodiments, the viral vector is conjugated to the first population of mesoporous silica particles. In some embodiments, the viral vector is electrostatically or covalently conjugated to the first population of mesoporous silica particles. In some embodiments, the first population of mesoporous silica particles are surface modified. In some embodiments, the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH₂—CH₂—)₁₋₂₅ linker. In some embodiments, the surface modification on the first population of mesoporous silica particles is a primary, secondary, tertiary, or quarternary amine. In some embodiments, the first population of mesoporous silica particles are surface modified with polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, the viral vector is a lentivirus, retrovirus, or adenovirus. In some embodiments, the composition further comprises a T cell stimulating compound or tumor antigen conjugated to or adsorbed on the first population of mesoporous silica particles or a second population of mesoporous silica particles. In some embodiments, the T cell stimulating compound or tumor antigen is conjugated to or adsorbed on the first population of mesoporous silica particles. In some embodiments in which the composition comprises the second population of mesoporous silica particles, the T cell stimulating compound or tumor antigen is conjugated directly to the second population of mesoporous silica particles or to a lipid envelope on the surface of the second population of mesoporous silica particles, and the T-cell stimulating compound or tumor antigen is IL-2, IL-15, anti-CD2 mAb, anti-CD3 mAb, anti-CD28 mAb, neo-antigen peptides, CD19, CD20, CD22, ROR1, mesothelin, CD33/IL3Ra, c-Met, PSMA, Glycolipid F77, EGFRvIII, GD-2, NY-ESO-1 TCR, MAGE A3 TCR, or combinations thereof. In some embodiments, the first or second population of mesoporous silica particles further comprises a cytokine conjugated to or adsorbed on the first or second population of mesoporous silica particles. In some embodiments, the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof. In some embodiments, the mesoporous silica particles are in the form of mesoporous silica rods.

Also contemplated herein is a method of delivering a viral vector to a desired site of action in a subject, comprising administering to the subject a composition comprising a first population of mesoporous silica particles and the viral vector. In some embodiments, the viral vector is conjugated to the first population of mesoporous silica particles. In some embodiments, the viral vector is electrostatically or covalently conjugated to the first population of mesoporous silica particles. In some embodiments, the first population of mesoporous silica particles are surface modified. In some embodiments, the surface modification on the first population of mesoporous silica particles is C₁₋₂₀ alkyl amine, C₁₋₂₀ carboxylic acid, C₁₋₂₀ azide, and substituted or unsubstituted C₁₋₂₀ alkyl. In some embodiments, the surface modification on the first population of mesoporous silica particles is a primary, secondary, tertiary, or quarternary amine. In some embodiments, the viral vector is a retrovirus, adenovirus, adeno-associated virus, herpes virus, or lentivirus. In some embodiments, the first population of mesoporous silica particles comprise pores of between 2-50 nm in diameter. In some embodiments, the first population of mesoporous silica particles have a surface area of at least about 100 m²/g. In some embodiments, the mesoporous silica particles are in the form of mesoporous silica rods.

Also contemplated herein is a method of expanding a chimeric antigen receptor (CAR) T (CAR-T) cell population, comprising contacting the CAR-T cell population with mesoporous silica particles conjugated to a targeting moiety, wherein the targeting moiety is complementary to the CAR. In some embodiments, the CAR is a protein engineered to target a tumor antigen. In some embodiments, the tumor antigen is selected from the group consisting of selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof. In some embodiments, the mesoporous silica particles are in the form of mesoporous silica rods.

Also contemplated herein is a composition comprising mesoporous silica particles conjugated to polyethylenimine. In some embodiments, the mesoporous silica particles are in the form of mesoporous silica rods. In some embodiments, the composition further comprising an active agent. In some embodiments, the active agent is absorbed or adsorbed on the mesoporous silica particles.

Also contemplated herein is a method of delivering an active agent to a desired site of action in a subject, comprising administering to the subject a composition comprising mesoporous silica particles conjugated to polyethylenimine and further comprising an active agent. In some embodiments, the active agent is absorbed or adsorbed on the mesoporous silica particles. In some embodiments, the composition provides sustained delivery of the active agent to the subject.

Also contemplated herein is a method of treating a subject having a disease, disorder, or condition, the method comprising: administering to the subject a composition comprising mesoporous silica particles conjugated to polyethylenimine and further comprising an active agent. In some embodiments, the active agent is absorbed or adsorbed on the mesoporous silica particles. In some embodiments, the disease, disorder, or condition is associated with a tumor antigen.

Also contemplated herein is a composition comprising mesoporous silica particles conjugated to polyethylenimine and further comprising an active agent, for use in a method of treating a subject having a disease, disorder, or condition. In some embodiments, the active agent is absorbed or adsorbed on the mesoporous silica particles. In some embodiments, the disease, disorder, or condition is associated with a tumor antigen. Also contemplated herein is a composition comprising a cell manufactured as described herein for use in a method of treating a subject having a disease, disorder, or condition. In some embodiments, the disease, disorder, or condition is associated with a tumor antigen.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents a series of surface modifications on mesoporous silica particles.

FIG. 2 presents results from staining for viral envelope protein (VSV-G) on MSR surface after adsorption of VSV-G pseudotyped lentivirus onto MSRs. The control MSRs are presented on the top panel and virus-incubated rods are on the bottom panel.

FIG. 3 is a schematic of virus adsorption on MSRs and transduction of T cells.

FIG. 4 provides results from GFP expression by T cells incubated with free lentivirus or MSR-bound lentivirus. Dilution of virus-coated MSRs from 40 μg/ml starting concentration is as indicated. The “1× lenti” condition is equivalent to the amount of virus incubated with the MSR conditions. The “2× lenti” condition is equivalent to twice the amount used to coat the MSR conditions.

FIG. 5 provides a schematic of overall strategy for ligand presentation on MSR surface. Liposomes are incubated with MSRs to form a supported lipid bilayer. Ligands can be coupled to the MSR-lipid bilayer using streptavidin-biotin interactions.

FIG. 6 shows a picture of MSRs coated with POPC liposomes containing 1 mol % PE-carboxyfluorescein. Bright field (left), fluorescence (middle), and overlay (right) images are shown.

FIG. 7 depicts the peptide sequence of EGFRvIII CAR-binding peptide (LEEKKGNYVVTDH (SEQ ID NO: 674)).

FIG. 8 illustrates cytokine production of EGFRvIII CARTs by peptide immobilization on MSRs. Results provide interferon-gamma and interleukin-2 production of EGFRvIII CARTs stimulated by lipid-coated MSRs (1% PE-biotin in the lipid coating) presenting EGFRvIII-CAR binding peptide compared to control conditions control conditions.

FIG. 9 illustrates the proliferation of EGFRvIII CARTs by peptide immobilization on MSRs. A lipid-coated MSR composition of 0.01% PE-biotin was used for peptide immobilization, and the MSR concentration was 30 μg/ml in the well. Cell counts were performed at day 7 of culture under the indicated conditions.

FIGS. 10A and 10B illustrate the proliferation of EGFRvIII CARTs and final cellular composition by peptide immobilization on MSRs. The starting MSR concentration was 50 μg/ml with and the dilutions of MSRs from this starting concentration are as indicated in the axis. FIG. 10A: Percentage of CD4 and CD8 T cells at the end of culture period with the indicated materials. FIG. 10B: FACS analysis of CD8+ and CD4+ CART cells diluting CFSE during a 3 day culture period using MSRs with varying amount of EGFRvIII CAR-binding peptide with or without anti-CD28 on the MSR surface.

FIGS. 11A and 11B illustrate the proliferation of BCMA CARTs and final cellular composition by BCMA protein immobilization on MSRs. The starting MSR concentration was 50 μg/ml with and the dilutions of MSRs from this starting concentration are as indicated in the axis. FIG. 11A: Percentage of CD4 and CD8 T cells at the end of culture period with the indicated materials. FIG. 11B: FACS analysis of CD8+ and CD4+ CART cells diluting CFSE during a 3 day culture period using MSRs with varying amount of EGFRvIII CAR-binding peptide with or without anti-CD28 on the MSR surface.

FIG. 12 presents a schematic of simultaneous stimulation and transduction of unstimulated human T cells using MSRs, according to some embodiments. Two populations of MSRs are created—1) MSRs presenting agonistic CD3/CD28 antibodies to stimulate T cells, 2) Positively charged PEI-MSRs that have been bound with lentivirus to facilitate viral delivery to the T cells. The two types of MSRs can be mixed together in different ratios to adjust the amount of stimulation and virus that the T cells are exposed to.

FIG. 13 illustrates the transduction efficiency of T cells exposed to stimulatory (anti-CD3/CD28 antibody-immobilized MSRs) and PEI-MSRs incubated with virus. T cells were incubated with different amounts of stimulating rods (Stim 1.00 represents 70 μg/ml MSRs) and exposed to GFP-lentivirus at different multiplicities of infection (MOI) either bound to PEI-MSRs or in free virus form. The top concentration of MSRs in the virus conditions was 22 μg/ml.

FIG. 14 illustrates transduction efficiency of T cells exposed to stimulatory (anti-CD3/CD28 antibody-immobilized) MSRs and PEI-MSRs incubated with virus. Plots show transduction efficiency as a function of the concentration of stimulatory MSRs at various total amounts of virus. The MSR concentration of stimulating MSR condition 1.0 is 70 μg/ml. The concentration of MSRs in the PEI MSR condition 1 is 22 pg/ml. Transduction was assessed at 3 days after initiation of the culture.

FIG. 15 provides results from comparison of virus delivery strategies for transduction efficiency. In the “PEI” and “free” conditions T cells were stimulated with a “high” level of CD3/CD28 antibodies bound to MSRs (MSR concentration 70 μg/ml), and given virus either associated with PEI-MSRs or freely delivered in the media, respectively (virus concentration 1.0 contains 22 μg/ml MSRs, MOI ˜6.7). In the “PEI+CD3/CD28” the virus and CD3/CD28 agonistic antibodies were bound to PEI-MSRs (concentration 1.0 is 22 μg/ml MSRs). Transduction was assessed at 3 days after initiation of the culture.

FIG. 16 provides results from comparison of various delivery strategies for transduction in PBMC population. Conditions as in FIG. 15 were added to PBMCs. The proportion of transduced cells in each cell type was quantified. Transduction was assessed at 3 days after initiation of the culture.

FIG. 17 provides the different transduction fractions in PBMCs with various virus delivery strategies. Top panel provides the total cell composition present in PBMC populations under the conditions of FIG. 15. Bottom panel provides the composition of the transduced cell fraction present after virus delivery using the conditions of FIG. 15. Transduction was assessed at 3 days after initiation of the culture.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In some aspects, the cytoplasmic signaling domain comprises a primary signaling domain (e.g., a primary signaling domain of CD3-zeta). In some aspects, the cytoplasmic signaling domain further comprises one or more functional signaling domains derived from at least one costimulatory molecule as defined below. In some aspects, the costimulatory molecule is chosen from 41BB (i.e., CD137), CD27, ICOS, and/or CD28. In some aspects, the CAR comprises a chimeric fusion protein comprising an extracellular antigen recognition domain, a transmembrane domain and an intracellular signaling domain comprising a functional signaling domain derived from a stimulatory molecule. In some aspects, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In some aspects, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In some aspects, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In some aspects the CAR comprises an optional leader sequence at the amino-terminus (N-ter) of the CAR fusion protein. In some aspects, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMA CAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab, F(ab)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

The portion of the CAR of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some aspects, the antigen binding domain of a CAR composition of the invention comprises an antibody fragment. In a further aspect, the CAR comprises an antibody fragment that comprises a scFv. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof.

As used herein, the term “binding domain” or “antibody molecule” refers to a protein, e.g., an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response, therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be synthesized or can be derived from a biological sample, or might be a macromolecule besides a polypeptide. Such a biological sample can include, but is not limited to a tissue sample, a tumor sample, a cell or a fluid with other biological components.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that it has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of a tumor antigen as described herein” includes, but is not limited to, a disease associated with expression of a tumor antigen as described herein or condition associated with cells which express a tumor antigen as described herein including, e.g., proliferative diseases such as a cancer or malignancy or a precancerous condition such as a myelodysplasia, a myelodysplastic syndrome or a preleukemia; or a noncancer related indication associated with cells which express a tumor antigen as described herein. In some aspects, a cancer associated with expression of a tumor antigen as described herein is a hematological cancer. In some aspects, a cancer associated with expression of a tumor antigen as described herein is a solid cancer. Further diseases associated with expression of a tumor antigen described herein include, but not limited to, e.g., atypical and/or non-classical cancers, malignancies, precancerous conditions or proliferative diseases associated with expression of a tumor antigen as described herein. Non-cancer related indications associated with expression of a tumor antigen as described herein include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In some embodiments, the tumor antigen-expressing cells produce the tumor antigen protein (e.g., wild-type or mutant), and the tumor antigen protein may be present at normal levels or reduced levels. In some embodiments, the tumor antigen-expressing cells produced detectable levels of a tumor antigen protein at one point, and subsequently produced substantially no detectable tumor antigen protein.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In some aspects, the signal is a primary signal that is initiated by, for instance, binding of a TCR/CD3 complex with an MEW molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing-cytoplasmic signaling sequence that is of particular use in the invention include, but are not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In a specific CAR of the invention, the intracellular signaling domain in any one or more CARS of the invention comprises an intracellular signaling sequence, e.g., a primary signaling sequence of CD3-zeta. In a specific CAR of the invention, the primary signaling sequence of CD3-zeta is the sequence provided as SEQ ID NO: 18, or the equivalent residues from a non-human species, e.g., mouse, rodent, monkey, ape and the like. In a specific CAR of the invention, the primary signaling sequence of CD3-zeta is the sequence as provided in SEQ ID NO:20, or the equivalent residues from a non-human species, e.g., mouse, rodent, monkey, ape and the like.

The term “antigen presenting cell” or “APC” refers to an immune system cell such as an accessory cell (e.g., a B-cell, a dendritic cell, and the like) that displays a foreign antigen complexed with major histocompatibility complexes (MHC on its surface. T-cells may recognize these complexes using their T-cell receptors (TCRs). APCs process antigens and present them to T-cells.

An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In some embodiments, the cytoplasmic domain of zeta comprises residues 52 through 164 of GenBank Acc. No. BAG36664.1 or a variant thereof (e.g., a molecule having mutations, e.g., point mutations, fragments, insertions, or deletions). In some embodiments, the “zeta stimulatory domain” or a “CD3-zeta stimulatory domain” is the sequence provided as SEQ ID NO: 9 or 10, or a variant thereof (e.g., a molecule having mutations, e.g., point mutations, fragments, insertions, or deletions). Alternatively or in addition, the term “zeta” or alternatively “zeta chain”, “CD3-zeta” (or “CD3zeta, CD3 zeta or CD3z) or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In some aspects the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In some aspects, the “zeta stimulatory domain” or a “CD3-zeta stimulatory domain” is the sequence provided as SEQ ID NO:18. In some aspects, the “zeta stimulatory domain” or a “CD3-zeta stimulatory domain” is the sequence provided as SEQ ID NO:20.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that specifically binds with CD83, and the like.

The intracellular signaling domain can comprise the entire intracellular portion, or the entire native intracellular signaling domain, of the molecule from which it is derived, or a functional fragment or derivative thereof.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or inhibition of growth or proliferation, of a target cell. In the case of a T cell, primary stimulation and co-stimulation are examples of immune effector function or response.

The term “encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising an expression control sequence operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses, “viral vectors”) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab, F(ab)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive promoter” refers to a nucleotide sequence which, when operably linked with a polynucleotide which encodes or specifies a gene product, causes the gene product to be produced in a cell under most or all physiological conditions of the cell.

The term “inducible promoter” refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In a preferred embodiment of a construct for transient expression, the polyA is between 50 and 5000 (SEQ ID NO: 34), preferably greater than 64, more preferably greater than 100, most preferably greater than 300 or 400. poly(A) sequences can be modified chemically or enzymatically to modulate mRNA functionality such as localization, stability or efficiency of translation.

As used herein in connection with expression, e.g., expression of a CAR molecule, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a binding partner (e.g., a tumor antigen) protein present in a sample, but which antibody or ligand does not substantially recognize or bind other molecules in the sample.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” as used herein refers to the return of a disease (e.g., cancer) or the signs and symptoms of a disease such as cancer after a period of improvement, e.g., after prior treatment of a therapy, e.g., cancer therapy

A “system” as the term is used herein in connection with, for example, gene editing, refers to a group of molecules, e.g., one or more molecules, which together act to produce a desired function.

A “gene editing system” as the term is used herein, refers to a system, e.g., one or more molecules, that direct and effect an alteration, e.g., a deletion, of one or more nucleic acids at or near a site of genomic DNA targeted by said system. Gene editing systems are known in the art, and are described more fully below.

A “dominant negative” gene product or protein is one that interferes with the function of a gene product or protein. The gene product affected can be the same or different from the dominant negative protein. Dominant negative gene products can be of many forms, including truncations, full length proteins with point mutations or fragments thereof, or fusions of full length wild type or mutant proteins or fragments thereof with other proteins. The level of inhibition observed can be very low. For example, it may require a large excess of the dominant negative protein compared to the functional protein or proteins involved in a process in order to see an effect. It may be difficult to see effects under normal biological assay conditions.

The term “proportion” refers to the ratio of the specified molecule to the total number of molecules in a population. In an exemplary embodiment, a proportion of T cells having a specific phenotype (e.g., T_{SCM} cells) refers to the ratio of the number of T cells having that phenotype relative to the total number of T cells in a population. In an exemplary embodiment, a proportion of T cells having a specific phenotype (e.g., CD45RA+CD62L+ cells) refers to the ratio of the number of T cells having that phenotype relative to the total number of T cells in a population. It will be understood that such proportions may be measured against certain subsets of cells, where indicted. For example, the proportion of CD4+T_{SCM} cells may be measured against the total number of CD4+ T cells.

The term “population of immune effector cells” as used herein refers to a composition comprising at least two, e.g., two or more, e.g., more than one, immune effector cell, and does not denote any level of purity or the presence or absence of other cell types. In an exemplary embodiment, the population is substantially free of other cell types. In another exemplary embodiment, the population comprises at least two cells of the specified cell type, or having the specified function or property.

The terms “T_{SCM}-like cell,” “naive T Cell’ and “naïve T cell” are used interchangeably and refer to a less differentiated T cell state, that is characterized by surface expression of CD45RA and CD62L (e.g., is CD45RA positive and CD62L positive (sometimes written as CD45RA+CD62L+)). In general, T cell differentiation proceeds, from most “naive” to most “exhausted,” T_{SCM}-like (e.g., a CD45RA+CD62L+ cell)>T_{CM} (e.g., a CD45RA-CD62L+ cell)>T_{EM} (e.g., a CD45RA-CD62L− cell)>T_{EFF}. Naive T cells may be characterized, for example, as having increased self-renewal, anti-tumor efficacy, proliferation and/or survival, relative to a more exhausted T cell phenotype. In an exemplary embodiment, a naive T cell refers to a CD45RA+CD62L+ T cell. In another exemplary embodiment, a naive T cell refers to a T_{SCM} cell, e.g., a CD45RA+CD62L+CCR7+CD27+CD95+ T cell.

The term “T_{SCM}” refers to a T cell having a stem cell memory phenotype, characterized in that it expresses CD45RA, CD62L, CCR7, CD27 and CD95 on its cell surface (e.g., is CD45RA positive, CD62L positive, CCR7 positive, CD27 positive and CD95 positive (sometimes written as CD45RA+CD62L+CCR7+CD27+CD95+)). A T_{SCM} cell is an example of a naive T cell. The T cell may be CD4+ and/or CD8+ T cell.

As used herein, the term “alkyl” refers to a fully saturated branched or unbranched (or straight chain or linear) hydrocarbon moiety, comprising 1 to 20 carbon atoms. Preferably the alkyl comprises 1 to 6 carbon atoms, and more preferably 1 to 4 carbon atoms. Representative examples of alkyl include methyl, ethyl, n-propyl, iso-propyl, n-butyl, sec-butyl, iso-butyl, vert-butyl, n-pentyl, isopentyl, neopentyl, n-hexyl, 3-methylhexyl, 2,2-dimethylpentyl, 2,3-dimethylpentyl, n-heptyl. For example, the term “C₁₋₆ alkyl” refers to a hydrocarbon having from one to six carbon atoms, and the term “C₁₋₇ alkyl” refers to a hydrocarbon having from one to seven carbon atoms.

As used herein, the term “haloalkyl” refers to an alkyl as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. Preferably, the polyhaloalkyl contains up to 12, or 10, or 8, or 6, or 4, or 3, or 2 halo groups. Representative examples of haloalkyl are fluoromethyl, difluoromethyl, trifluoromethyl, chloromethyl, dichloromethyl, trichloromethyl, pentafluoroethyl, heptafluoropropyl, difluorochloromethyl, di chlorofluoromethyl, difluoroethyl, difluoropropyl, dichloroethyl and dichloropropyl. A perhaloalkyl refers to an alkyl having all hydrogen atoms replaced with halo atoms. For example, the term “halo-C₁₋₆ alkyl” refers to a hydrocarbon having one to six carbon atoms and being substituted by one or more halo groups, and the term “halo-C₁₋₇ alkyl” refers to a hydrocarbon having one to seven carbon atoms and being substituted by one or more halo groups.

As used herein, “salts” includes pharmaceutically acceptable acid addition salts that can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another.

All publications, patent applications, patents, and other references mentioned herein are incorporated by reference in their entirety.

Other features, objects, and advantages of the invention will be apparent from the description and drawings, and from the claims.

Surface Modified Mesoporous Silica Particles

In some embodiments, the invention provides mesoporous silica particles. Mesoporous silica particles comprise a porous body, for example, with hexagonal close-packed, cylinder-shaped, uniform pores. Mesoporous silica particles can be synthesized by using a rod-like micelle of a surfactant as a template, which is formed in water by dissolving and hydrolyzing a silica source such as alkoxysilane, sodium silicate solution, kanemite, silica fine particle in water or alcohol in the presence of acid or basic catalyst. See, e.g., US Pub. No. 2015-0072009 and Hoffmann et al., Angewandte Chemie International Edition, 45, 3216-3251, 2006. Many kinds of surfactants such as cationic, anionic, and nonionic surfactants have been examined as the surfactant and it has been known that generally, an alkyl trimethylammonium salt of cationic surfactant leads to a mesoporous silica having the greatest specific surface area and a pore volume. See, U.S. Publication No. 2013/0052117 and Katiyar et al. (Journal of Chromatography 1122 (1-2): 13-20).

The mesoporous silica particles may be provided in various forms, e.g., microspheres, irregular particles, rectangular rods, round nanorods. The mesoporous silica particles can have various predetermined shapes, including, e.g., a spheroid shape, an ellipsoid shape, a rod-like shape, or a curved cylindrical shape. In particular embodiments, the compositions and methods recited herein use mesoporous silica rods (MSR). Methods of assembling mesoporous silica to generate microrods are known in the art. See, Wang et al, Journal of Nanoparticle Research, 15: 1501, 2013. In some embodiments, mesoporous silica particles are synthesized by reacting tetraethyl orthosilicate with a template made of micellar rods. The result is a collection of mesoporous silica spheres or rods that are filled with a regular arrangement of pores. The template can then be removed by washing with a solvent adjusted to the proper pH. In this example, after removal of surfactant templates, the mesoporous silica particles are characterized by a uniform, ordered, and connected mesoporosity are prepared with a specific surface area of, for example, about 600 m²/g to about 1200 m²/g, particularly about 800 m²/g to about 1000 m²/g and especially about 850 m²/g to about 950 m²/g. In another embodiment, the mesoporous silica particles may be synthesized using a sol-gel method or a spray drying method. Tetraethyl orthosilicate is also used with an additional polymer monomer (as a template). In yet another embodiment, one or more tetraalkoxy-silanes and one or more (3-cyanopropyl)trialkoxy-silanes may be co-condensed to provide the mesoporous silicate particles as rods. See, US Publication Nos. 2013-0145488, 2012-0264599 and 2012-0256336, the content of which are incorporated by reference in their entireties.

The mesoporous silica particles (MSPs) (e.g., MSRs) may comprise pores, which may be ordered or randomly distributed, of between 2 to 100 nm in diameter, or 2-50 nm in diameter, e.g., pores of between 2-5 nm, 10-20 nm, 10-30 nm, 10-40 nm, 20-30 nm, 30-50 nm, 30-40 nm, 40-50 nm. In some embodiments, the microrods comprise pores of approximately 5 nm, 6 nm, 7 nm, 8 nm, 9 nm, 10 nm, 11 nm, 12 nm, or more in diameter. The pore size may be altered depending on the type of application.

In some embodiments, the length of the MSRs is in the micrometer range, ranging from about 5 μm to about 500 μm. In one example, the MSRs comprise a length of 5-50 μm, e.g., 10-20 μm, 10-30 μm, 10-40 μm, 20-30 μm, 30-50 μm, 30-40 μm, 40-50 μm. In some embodiments, the MSRs comprise length of 50 μm to 250 μm, e.g., about 60 μm, 70 μm, 80 μm, 90 μm, 100 μm, 120 μm, 150 μm, 180 μm, 200 μm, 225 μm, or more. In some embodiments, the MSRs having a higher aspect ratio, e.g., with rods comprising a length of 50 μm to 200 μm, particularly a length of 80 μm to 120 μm, especially a length of about 100 μm or more, are used.

In yet another embodiment, the MSPs (e.g., MSRs) provide a high surface area for attachment and/or binding to target cells, e.g., T-cells. Methods of obtaining high surface area mesoporous silicates are known in the art. See, e.g., U.S. Pat. No. 8,883,308 and US Publication No. 2011-0253643, the entire contents of which are incorporated by reference herein. In some embodiments, the high surface area is due to the fibrous morphology of the nanoparticles, which makes it possible to obtain a high concentration of highly dispersed and easily accessible moieties on the surface. In certain embodiments, the high surface area MSPs (e.g., MSRs) have a surface area of at least about 100 m²/g, at least 150 m²/g, or at least 300 m²/g. In other embodiments, the high surface area MSPs (e.g., MSRs) have a surface area from about 100 m²/g to about 1000 m²/g, including all values or sub-ranges in between, e.g., 50 m²/g, 100 m²/g, 200 m²/g, 300 m²/g, 400 m²/g, 600 m²/g, 800 m²/g, 100-500 m²/g, 100-300 m²/g, 500-800 m²/g or 500-1000 m²/g.

In some embodiments, the mesoporous silica particles may include a surface modification. As used herein, “surface modification” means attaching or appending functional groups on to the surface of the MSPs (e.g., MSRs). In some embodiments, the functional groups are adsorbed or covalently bonded onto the surfaces lining the pores and/or nanochannels or the surface of the MSPs (e.g., MSRs). As used herein, the “functional group” defines a chemical moiety linked to the MSR. In some embodiments, the functional group is a —OH (hydroxyl), amine, carboxylic acid, phosphonate, halide, azide, alkyne, epoxide, sulfhydryl, disulfide, polyethyleneimine, hydrophobic moiety, or salts thereof. In some embodiments, the functional group (i.e. —OH (hydroxyl), amine, carboxylic acid, phosphonate, halide, azide, alkyne, epoxide, sulfhydryl, disulfide, polyethyleneimine, hydrophobic moiety, or salts thereof) may be separated from the silica surface by a linker. In some embodiments, the functional group is covalently bonded to the MSP or MSR surface via a C₁ to C₂₀ alkyl linker. In other embodiments, the functional group is covalently bonded to the MSP or MSR surface via a polyethyleneglycol linker. In particular embodiments, the polyethylene glycol linker has the formula (—O(CH₂—CH₂—)₁₋₂₅. In particular embodiments, the surface modification is a C₁ to C₂₀ alkyl perhaloalkyl or a C₁ to C₂₀ alkyl perfluoroalkyl.

A general structure of surface modifications may be as follows:

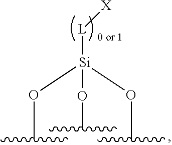

wherein L is a linker, and

X is a functional group.

In some embodiments, L may be C₁ to C₂₀ alkyl group or a polyethylene glycol group, and X may be —OH (hydroxyl), primary, secondary, tertiary or quarternary amine, carboxylic acid, phosphonate, halide, azide, alkyne, epoxide, sulfhydryl, disulfide, polyethyleneimine, or hydrophobic moiety, or salts thereof.

As used herein, surface modification having a phosphonate (also known as phosphonate-modified nanoparticles) have at least one phosphonic acid (—P(O)(OH)₂) group or phosphinic acid (—P(O)(OH)R, where R is an C₁ to C₂₀ alkyl group). The phosphonic or phosphinic acid may be charged or uncharged, depending on the pH. At physiological pH, phosphonic acids and phosphinic acids are negatively charged, or anionic. Phosphonate modifications may be prepared, for example, by treating the silica body surface with a phosphonate bearing trialkylsiloxane compound or phosphonate-bearing trihydroxylsilyl compound, such as (trihydroxylsilyl)propyl methylphosphonate.

In some embodiments, the mesoporous silica particles (e.g., MSRs) are surface modified with a primary, secondary, tertiary, or quarternary amine. Secondary, tertiary, and quarternary amines may be substituted with C₁ to C₂₀ alkyl groups and may be charged. In some embodiments, the amine group may be in the salt form. In some embodiments, the primary, secondary, tertiary, or quarternary amine may be separated from the MSP surface by a linker. In particular embodiments, the mesoporous silica particles are modified with polyethyleneimine. In specific embodiments, the polyethyleneimine is branched or unbranched. In alternate embodiments, the polyethyleneimine group has an average molecular weight in the range of about 1000 to 100,000 Daltons (Da), as measured by gel permeation chromatography (GPC). In some embodiments, the polyethyleneimine group has an average molecular weight of about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, about 10,000 Da, or about 20,000 Da, as measured by gel permeation chromatography (GPC).

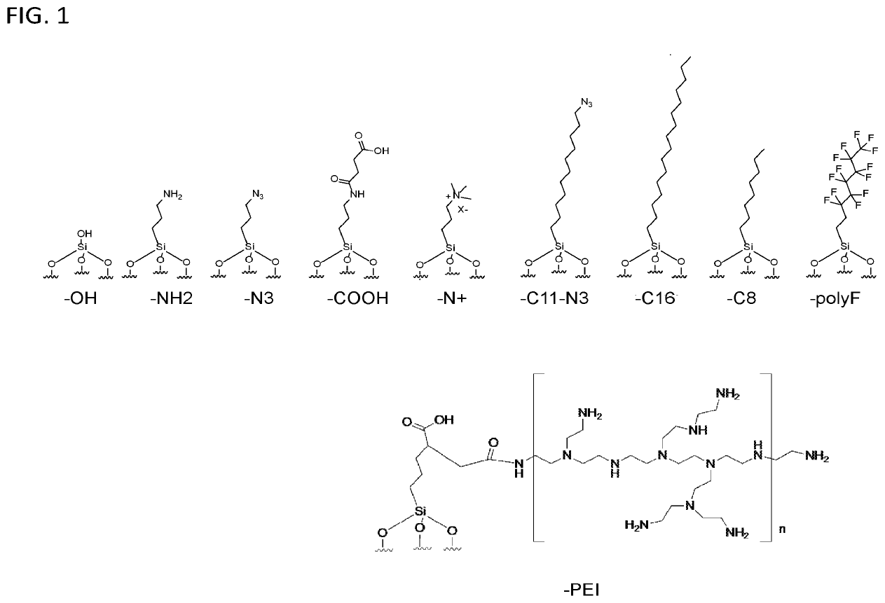

Structures of various exemplary surface modified mesoporous silica particles are shown in FIG. 1.

The MSPs (e.g., MSRs) recited herein are prepared by methods known to those of skill in the art, as noted herein. Generally, MSPs with surface modification may be prepared by the following method.

In general, any reaction capable of reacting with the silyl hydroxide surface of the MSPs (e.g., MSRs) may be used to covalently modify the surface. For example, the surface of the MSP (e.g. MSR) may be treated with a trialkoxysilyl compound or trihydroxysilyl compound. In some embodiments mesoporous silica particles are suspended in a suitable reaction solvent. In some embodiments, the reaction solvent may be aqueous solvents or buffers of a pH from 0-14. Additional co-mixture of aqueous solutions with 1 or more organic solvents, including but not limited to tetrahydrofuran, 2-methyl tetrahydrofuran, ethyl acetate, toluene, triethylamine, dimethylformamide, dimethylacetamide, dimethylsulfoxide, methanol, ethanol, methylene chloride, or dichloroethane, may be used. In some embodiments, the suspended mesoporous silica particles are reacted with a trialkoxysilyl or trihydroxysilyl reagent having the desired functional group as described herein. Amine modifications may be prepared, for example, by treating the MSPs with an amine bearing trialkoxysilane compound, such as aminopropyltriethoxysilane, 3-(2-aminoethylamino)propyl-trimethoxysilane, or 3-trimethoxysilylpropyl ethylenediamine. In certain embodiments, the trialkoxysilyl is a trimethoxysilyl or triethoxysilyl group. In alternate embodiments, the trialkoxysilyl reagent is a trialkoxy alkylamine. In some embodiments, the trialkoxy alkylamine includes a primary, secondary, tertiary, or quarternary amine.

In certain embodiments, the trialkoxysilyl reagent includes a polyethyleneimine group. In specific embodiments, the polyethyleneimine is branched or unbranched. In alternate embodiments, the polyethyleneimine group has an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC). In some embodiments, the trialkoxysilyl reagent includes an C₁₋₂₀ alkylazide group. In certain embodiments, the trialkoxysilyl reagent includes an C₁₋₂₀ alkylcarboxylic acid group. In other embodiments, the trialkoxysilyl reagent includes a C₁₋₂₀ alkyl group.

A sulfhydryl modification on a MSP (e.g., MSR) may be prepared, for example, by treating the MSP with a sulfhydryl bearing trialkoxysilane compound, such as 3-mercaptopropyltriethoxysilane.

A disulfide modification on a MSP (e.g., MSR) may be prepared, for example, by treating the surface of the nanoparticle with a disulfide bearing trialkoxysilane compound, or by treating a sulfhydryl modified surface with 2,2′-dithiodipyridine or other disulfide.

MSP (e.g., MSR) surface modifications to include a carboxylic acid group may be prepared, for example, by treating the surface with a carboxylic acid bearing trialkoxysilane compound, or by treating the MSP with a trialkoxysilane compound bearing a functional group that may be converted chemically into a carboxylic acid. For example, the MSP may be treated with 3-cyanopropyltriethoxysilane, followed by hydrolysis with sulfuric acid.

MSP (e.g., MSR) surface modifications to include an epoxide will have at least one epoxide may be prepared, for example, by treating the MSP with an epoxide bearing trialkoxysilane compound, such as glycidoxypropyltriethoxysilane.

Surface modifications having a hydrophobic moiety will have at least one moiety intended to reduce the solubility in water, or increase the solubility in organic solvents. Examples of hydrophobic moieties include long chain alkyl groups (e.g., C₈-C₂₀ alkyl groups), fatty acid esters (e.g., C₁-C₂₂ alkyl acid esters), and aromatic rings having C₆-C₁₀ carbon atoms.

In some embodiments, the reaction of the MSPs (e.g., MSRs) with the trialkoxysilyl reagent is carried out at ambient or room temperature. In other embodiments, the reaction is carried out at elevated temperatures. In further embodiments, the temperature of the reaction is from about 40° C. to about 120° C., about 50° C. to about 100° C., about 60° C. to about 80° C., about 70° C. to about 80° C., or about 50° C., about 55° C., about 60° C., about 65° C., about 70° C., about 75° C., about 80° C., about 85° C., about 90° C., about 95° C., or about 100° C.

Viral Vectors

In some embodiments, the compositions described herein can include mesoporous silica particles as described herein and viral vectors.

The virus vector can be any virus vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. By way of example, the virus vector can be an adenovirus, a lentivirus, a retrovirus, an adeno-associated virus, or a herpesvirus. In some embodiments, the virus vector is a lentivirus vector or an adenovirus vector.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CARs can be accomplished using of transposons such as sleeping beauty, CRISPR, CAS9, and zinc finger nucleases. See June et al. 2009 Nature Reviews Immunology 9.10: 704-716, is incorporated herein by reference.

In some embodiments, the viral vector comprises an expression vector comprising a recombinant polynucleotide comprising an expression control sequence operatively linked to a nucleotide sequence to be expressed. In some embodiments, the nucleotide sequence expresses a chimeric antigen receptor (CAR), engineered TCR, cytokines, chemokines, shRNA to block an inhibitory molecule, or mRNA to induce expression of protein. In some embodiments, the protein is a CAR that comprises an antigen binding domain, a transmembrane domain, a costimulatory signaling region, and a signaling domain. In some embodiments, the signaling domain is a CD3 zeta signaling domain.

In some embodiments, the nucleotide sequence in the viral vector express a peptide engineered to target a tumor antigen. In some embodiments, the peptide targets a tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof. In some embodiments, the peptide is a chimeric antigen receptor (CAR) or an engineered TCR. Such peptides are described in greater detail herein below in the section entitled General Description of Chimeric Antigen Receptor Technology.

Compositions of Mesoporous Silica Particles and Viral Vectors

Also described herein is a composition, comprising a first population of mesoporous silica particles and a viral vector. In some embodiments, the MSPs (e.g., MSRs) further comprise a plurality of functional groups adsorbed or covalently bonded onto the surfaces lining the pores and/or nanochannels or the surface. In some embodiments, the functional group is a —OH (hydroxyl), amine, carboxylic acid, phosphonate, halide, azide, alkyne, epoxide, sulfhydryl, disulfide, polyethyleneimine, hydrophobic moiety or salts thereof. In some embodiments, the functional group (i.e. —OH (hydroxyl), amine, carboxylic acid, phosphonate, halide, azide, alkyne, epoxide, sulfhydryl, disulfide, polyethyleneimine, hydrophobic moiety, or salts thereof) may be may be directly attached to the surface of the MSP. In some embodiments, the functional group is covalently bonded to the MSP (e.g., MSR) surface via a C₁ to C₂₀ alkyl linker. In other embodiments, the functional group is covalently bonded to the MSP surface via a polyethyleneglycol linker. In particular embodiments, the polyethylene glycol linker has the formula (—O(CH₂—CH₂—)₁₋₂₅. In particular embodiments, the surface modification is a C₁ to C₂₀ alkyl perhaloalkyl or a C₁ to C₂₀ alkyl perfluoroalkyl.

In some embodiments, the MSPs (e.g., MSRs) are surface modified with a primary, secondary, tertiary, or quarternary amine. In particular embodiments, the mesoporous silica rods are modified with polyethyleneimine. In specific embodiments, the polyethyleneimine is branched or unbranched. In alternate embodiments, the polyethyleneimine group has an average molecular weight in the range of about 1000 to 20,000 Daltons (Da), as measured by gel permeation chromatography (GPC). In some embodiments, the polyethyleneimine group has an average molecular weight of about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC).

In some embodiments, the viral vector is conjugated to the mesoporous silica particles. As described herein, “conjugated to” means associated with or attached to by any means as described herein. In some embodiments, the viral vector is electrostatically or covalently conjugated to the mesoporous silica particles. In some embodiments, the electrostatic conjugation between the mesoporous silica particles and the viral vector is due to oppositely surface-charged viral vectors and mesoporous silica particles. For example and without being bound by theory, mesoporous silica particles that are surface modified by polyethyleneimine or primary, secondary, tertiary, or quarternary ammonium groups that are positively charged can be conjugated to negatively surface-charged viral vectors. Thus in some embodiments, the viral vector is negatively charged and the mesoporous silica particles are positively charged. In some embodiments, the covalent conjugation between the mesoporous silica particles and the viral vector is achieved by methods known to those of skill in the art, either via linkers or without linkers. For example and without limitation, the linkers may be polyethylene glycol, alkyl groups, polymers, polyamide linkages, etc.

In some aspects, provided herein includes pharmaceutical compositions comprising mesoporous silica particles as described herein, formulated for use in the manufacture of a population of immune effector cells, e.g., T lymphocyte cells. In some embodiments, the T lymphocyte cells are transduced with a CAR. In some embodiments, the MSPs are conjugated to a viral vector as described herein. In some embodiments, the MSPs for use in in the manufacture of a population of immune effector cells, e.g., T lymphocyte cells, may be surface modified as described herein.

In some embodiments, the composition is suitable for use as an injectable composition comprising mesoporous silica particles and a viral vector, wherein the viral vector comprises an expression vector comprising a recombinant polynucleotide comprising an expression control sequence operatively linked to a nucleotide sequence to be expressed. In some embodiments, the viral vector is conjugated to the mesoporous silica particles as described herein.

In compositions of mesoporous silica particles described herein, the MSPs (e.g., MSRs) may be present in a concentration of 0.01 to 1000 μg/ml. In alternative embodiments, the concentration of MSPs or MSRs in the compositions described herein may be 0.1 to 500 μg/ml, 0.5 to 100 μg/ml, 1 to 90 μg/ml, 1 to 80 μg/ml, 1 to 70 μg/ml, 1 to 60 μg/ml, 1 to 50 μg/ml, or 1 to 40 μg/ml.

In particular embodiments, the MSPs (e.g., MSRs) may be present in a concentration of about 1 μg/ml, 10 μg/ml, 20 μg/ml, 30 μg/ml, 40 μg/ml, 50 μg/ml, 60 μg/ml, 70 μg/ml, 80 μg/ml, 90 μg/ml, 100 μg/ml, 110 μg/ml, 120 μg/ml, 130 μg/ml, 140 μg/ml, or 150 μg/ml.

In general, compositions described herein may be administered in therapeutically effective amounts as described above via any of the usual and acceptable modes known in the art, either singly or in combination with one or more therapeutic agents.

Injectable compositions may be aqueous isotonic suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically effective substances.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

The pharmaceutical composition (or formulation) for application may be packaged in a variety of ways depending upon the method used for administering the compositions described herein.

Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

In some embodiments, the compositions described herein further include a T cell stimulating compound or tumor antigen. In some embodiments, the T cell stimulating compound or tumor antigen is conjugated to or adsorbed on the first population of mesoporous silica particles. In additional or alternative embodiments, the T cell stimulating compound or tumor antigen is conjugated to or adsorbed on to a second population of mesoporous silica particles. In further embodiments, the T-cell stimulating compound or tumor antigen is IL-2, IL-15, GM-CSF, anti-CD2 mAb, anti-CD3 mAb, anti-CD28 mAb, neo-antigen peptides, peptides from shared antigens such as TRP2, gp100, tumor cell lysate, CD19, CD20, CD22, ROR1, mesothelin, CD33/IL3Ra, c-Met, PSMA, Glycolipid F77, EGFRvIII, GD-2, NY-ESO-1 TCR, MAGE A3 TCR, or combinations thereof. Adsorption to the MSP (e.g., MSR) surface is as commonly understood as a molecule adhering to the surface.

In embodiments where the T cell stimulating compound or tumor antigen is conjugated to the second population of mesoporous silica particles, the T cell stimulating compound or tumor antigen may be conjugated to a lipid bilayer on the surface of the second population of mesoporous silica particles. Methods of making lipid bilayers on the mesoporous silica particles are known. See e.g., International Appl. Publ. No. WO 2018/013797. Briefly, liposomes containing predefined amounts of a label such as biotin are used to coat the MSPs. The labels may then be used to affix to the T-cell stimulating compound using a complementary label, e.g., streptavidin. Lipids used to make liposomes are known to those of skill in the art and include, without limitation, vesicle-forming lipids having two hydrocarbon chains, typically acyl chains, and a polar head group. Included in this class are the phospholipids, such as phosphatidylcholine (PC), phosphatidylethanolamine (PE), phosphatidic acid (PA), phosphatidylinositol (PI), and sphingomyelin (SM), where the two hydrocarbon chains are typically between about 14-22 carbon atoms in length, and have varying degrees of unsaturation. In some embodiments, the lipid is a relatively unsaturated phospholipid (having one, two or three double bonds in the hydrocarbon chain). In some embodiments, the lipid is a phosphatidylcholine.

Phosphatidylcholine is a phospholipid that incorporates choline as a headgroup and combines a glycerophosphoric acid with two fatty acids. In some embodiments, the phosphatidylcholine is a palmitoyl phosphatidylcholine or a oleoyl phosphatidylcholine or a 1-palmitoyl,2-oleoyl-phosphatidylcholine. More than one type of lipid may be used in preparing the liposome composition. The selection of lipids and proportions can be varied to achieve a desired degree of fluidity or rigidity, and/or to control stability. Where more than one type of lipid is used in preparing the liposome composition, a suitable amount of the relatively unsaturated lipid (such as PC) should be used in order to form stable liposomes. In some embodiments, at least 45-50 mol % of the lipids used in the formulation are PC. The liposomes may also include lipids derivatized with a hydrophilic polymer such as polyethylene glycol (PEG). Suitable hydrophilic polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethyl cellulose, polyethyleneglycol, polyaspartamide, and hydrophilic peptide sequences. Methods of preparing lipids derivatized with hydrophilic polymers are known (see e.g. U.S. Pat. No. 5,395,619, which is incorporated herein by reference).

In some embodiments, the first population or second population of mesoporous silica particles further includes a cytokine. The cytokine may be, without limitation, IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof. In particular embodiments, the cytokine is conjugated to or adsorbed on the first or second population of mesoporous silica particles. In embodiments, where the cytokine is adsorbed on the second population of mesoporous silica particles, the second population of MSPs (e.g., MSRs) may be further covered by a lipid bilayer, as described above.

Methods

In some embodiments, the invention relates to a method, comprising:

contacting T lymphocytes with a composition comprising a first population of mesoporous silica particles (e.g., MSRs) and a viral vector;

wherein the viral vector comprises an expression vector comprising the recombinant polynucleotide comprising an expression control sequence operatively linked to a nucleotide sequence to be expressed.

In some embodiments, the contacting occurs in vitro. In some embodiments, the T lymphocytes are activated before or after contacting with the mesoporous silica particles.

In some embodiments, the invention relates to a method of genetically transducing T lymphocytes with a recombinant polynucleotide in vivo, comprising:

administering to a subject, having one or more T lymphocytes, a composition comprising a first population of mesoporous silica particles (e.g., MSRs) and a viral vector;

wherein the viral vector comprises an expression vector comprising a recombinant polynucleotide comprising an expression control sequence operatively linked to a nucleotide sequence to be expressed, and

wherein when the composition contacts one or more T lymphocytes, the T lymphocytes are genetically transduced with the recombinant polynucleotide.

In some embodiments, the invention relates to a method of expanding a T lymphocyte population in vitro, comprising contacting the T lymphocyte population with:

(a) a composition comprising a first population of mesoporous silica particles (e.g., MSRs) and a viral vector to provide a transduced T lymphocyte population; and

(b) contacting the transduced T lymphocyte population with a T cell stimulating compound or tumor antigen and optionally, a cytokine;

wherein the viral vector comprises an expression vector comprising the recombinant polynucleotide comprising an expression control sequence operatively linked to a nucleotide sequence to be expressed.

In some embodiments of the presently described methods, the methods result in an increase in the proportion of T lymphocytes in the population.

In some embodiments, the invention relates to a method of expanding a chimeric antigen receptor (CAR) T cell population, comprising contacting the CAR-T cell population with mesoporous silica particles (e.g., MSRs) conjugated to a targeting moiety, wherein the targeting moiety is complementary to the CAR.

In some embodiments, the invention relates to a method of selectively expanding the proportion of T lymphocytes in a culture comprising contacting the T lymphocyte population with:

(a) a composition comprising a first population of mesoporous silica particles (e.g., MSRs) and a viral vector to provide a transduced T lymphocyte population; and

(b) contacting the transduced T lymphocyte population with a T cell stimulating compound or tumor antigen and optionally, a cytokine;

wherein the viral vector comprises an expression vector comprising the recombinant polynucleotide comprising an expression control sequence operatively linked to a nucleotide sequence to be expressed.

In some embodiments, the culture includes different effector cell types, including NK cells, monocytes, B cells. In particular embodiments, the proportion of T lymphocytes is enhanced by about 10%, 20%, 30%, 40%, or 50%, compared to the proportion of T lymphocytes prior to contacting with the MSP composition. In some embodiments, the population of cells is expanded for a period of 8 days or less.

In some embodiments, the invention is a method of delivering a viral vector to a desired site of action in a subject, comprising administering to the subject a composition comprising a first population of mesoporous silica particles and the viral vector. Compositions of mesoporous silica particles (e.g., MSRs) and the viral vector are as described above.

In some aspects, in the methods recited herein, the mesoporous silica particles can be surface modified with a plurality of functional groups adsorbed or covalently bonded onto the surfaces lining the pores and/or nanochannels or the surface of the MSPs (e.g., MSRs), as described herein. In some embodiments, the functional group is a —OH (hydroxyl), amine, carboxylic acid, phosphonate, halide, azide, alkyne, epoxide, sulfhydryl, disulfide, polyethyleneimine, hydrophobic moiety or salts thereof. In some embodiments, the functional group (i.e. —OH (hydroxyl), amine, carboxylic acid, phosphonate, halide, azide, alkyne, epoxide, sulfhydryl, disulfide, polyethyleneimine, hydrophobic moiety or salts thereof) may be separated from the silica surface by a linker. In some embodiments, the functional group is covalently bonded to the MSP or MSR surface via a C₁ to C₂₀ alkyl linker. In other embodiments, the functional group is covalently bonded to the MSP (e.g., MSR) surface via a polyethyleneglycol linker. In particular embodiments, the surface modification is a C₁ to C₂₀ alkyl perhaloalkyl or a C₁ to C₂₀ alkyl perfluoroalkyl.

In another aspect, in the methods described herein, the viral vector is as described herein. In the methods of the present invention, the viral vector may be conjugated to the mesoporous silica particles (e.g., MSRs) as described herein. In some embodiments, the electrostatic conjugation between the mesoporous silica particles and the viral vector is due to oppositely charged viral vectors and mesoporous silica particles. For example and without being bound by theory, mesoporous silica particles that are surface modified by polyethylene imine or primary, secondary, tertiary, or quarternary ammonium groups that are positively charged can be conjugated to negatively charged viral vectors. Thus in some embodiments, the viral vector is negatively charged and the surface modified mesoporous silica particles are positively charged. In some embodiments, the covalent conjugation between the mesoporous silica particles and the viral vector is achieved by methods known to those of skill in the art, either via linkers or without linkers. For example and without limitation, the linkers may be polyethylene glycol, alkyl groups, polymers, polyamide linkages, etc.

In some methods recited herein, the nucleotide sequence expresses a chimeric antigen receptor (CAR), engineered TCR, cytokines, chemokines, shRNA to block an inhibitory molecule, or mRNA to induce expression of a protein. In specific embodiments, the nucleotide sequence to be expressed expresses a CAR.

In some methods described herein, T lymphocytes may be activated by contacting the T lymphocytes with a T cell stimulating compound or tumor antigen. Examples of T-cell stimulating compounds are provided herein. In some embodiments, the T cell stimulating compound or tumor antigen is conjugated to or adsorbed on a first population of mesoporous silica particles or a second population of mesoporous silica particles, or both populations of MSPs (e.g., MSRs). In other embodiments, the T cell stimulating compound or tumor antigen is conjugated to or adsorbed on a first population of mesoporous silica particles. In particular embodiments, the T cell stimulating compound or tumor antigen is conjugated to directly to the second population of mesoporous silica particles or to a lipid envelope on the surface of the second population of mesoporous silica particles. Preparation of a lipid envelope on the surface of the MSPs is known and described herein. See e.g., International Appl. Publ. No. WO 2018/013797.

Methods described herein may further include contacting T lymphocytes with a cytokine. In some embodiments, the cytokine is in the medium with the MSPs (e.g., MSRs) or conjugated to or adsorbed on the first or second population or both populations of mesoporous silica particles. In some embodiments, the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof.

In some embodiments, the method further comprises expanding a population of T cells after transduction. The T cells/T lymphocytes can be expanded by the methods described herein. In some embodiments, the population of cells is expanded for a period of 8 days or less.

In yet other embodiments, the population of cells is expanded in vitro for 5 days, and the resulting cells exhibit higher proinflammatory IFN-γ and/or GM-CSF levels, as compared to the same cells expanded in culture for 9 days under the same culture conditions.

Without being bound by theory, it is believed that the methods described herein conserve lentivirus during the CAR-T cell manufacturing process. The stimulatory capabilities of the materials system allow unique capabilities from the currently used reagents for CAR T cell manufacturing by allowing antigen-specific stimulation of CAR T cells, which may enhance CAR T cell function when transferred into the body, or may be used to selectively stimulate and expand CAR T cells relative to non-CAR T cells in the cultures to enhance the purity of the CAR T cell product.

In some embodiments, the invention is a method of delivering an active agent to a desired site of action in a subject, comprising administering to the subject a composition comprising mesoporous silica particles conjugated to polyethyleneimine. In particular embodiments, the polyethyleneimine is covalently conjugated to the mesoporous silica particles. In some embodiments, the polyethyleneimine group has an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC).

In some embodiments, the invention provides a method of providing sustained drug delivery to a subject at a desired site of action, comprising administering to the subject a composition comprising mesoporous silica particles conjugated to polyethyleneimine, and an active agent absorbed or adsorbed on the mesoporous silica particles.

In some embodiments, the active agent is an anticancer drug.

General Description of Chimeric Antigen Receptor Technology

In some embodiments of the invention, described herein are methods for using mesoporous silica particles for the manufacture, e.g., the activation and/or expansion, a population of immune effector cells, e.g., T cells or NK cells, engineered to express a CAR molecule, e.g., as described herein, wherein the cells have enhanced activity (e.g., proliferation, cytokine release, and/or tumor targeting efficacy).

In some embodiments, the recombinant polypeptide construct encodes a chimeric antigen receptor (CAR) comprising an antigen binding domain (e.g., antibody or antibody fragment, TCR or TCR fragment) that binds to a tumor-supporting antigen (e.g., a tumor-supporting antigen as described herein), a transmembrane domain (e.g., a transmembrane domain described herein), and an intracellular signaling domain (e.g., an intracellular signaling domain described herein) (e.g., an intracellular signaling domain comprising a costimulatory domain (e.g., a costimulatory domain described herein) and/or a primary signaling domain (e.g., a primary signaling domain described herein). In some embodiments, the tumor-supporting antigen is an antigen present on a stromal cell or a myeloid-derived suppressor cell (MDSC). In other aspects, the invention features polypeptides encoded by such nucleic acids and host cells containing such nucleic acids and/or polypeptides.

In some embodiments, the nucleotide sequence in the vector expresses a protein engineered to target a tumor antigen.

In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

A CAR described herein can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation. Without wishing to be bound by theory, in some embodiments, the CAR-expressing cells destroy the tumor-supporting cells, thereby indirectly inhibiting tumor growth or survival.

In embodiments, the stromal cell antigen is chosen from one or more of: bone marrow stromal cell antigen 2 (BST2), fibroblast activation protein (FAP) and tenascin. In some embodiments, the FAP-specific antibody is, competes for binding with, or has the same CDRs as, sibrotuzumab. In embodiments, the MDSC antigen is chosen from one or more of: CD33, CD11b, C14, CD15, and CD66b. Accordingly, in some embodiments, the tumor-supporting antigen is chosen from one or more of: bone marrow stromal cell antigen 2 (BST2), fibroblast activation protein (FAP) or tenascin, CD33, CD11b, C14, CD15, and CD66b.

In some embodiments, the antigen binding domain of the encoded CAR molecule comprises an antibody, an antibody fragment, an scFv, a Fv, a Fab, a (Fab′)2, a single domain antibody (SDAB), a VH or VL domain, a camelid VHH domain or a bi-functional (e.g. bi-specific) hybrid antibody (e.g., Lanzavecchia et al., Eur. J. Immunol. 17, 105 (1987)).

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO: 22). In some embodiments, the linker can be (Gly₄Ser)₄ (SEQ ID NO:29) or (Gly₄Ser)₃ (SEQ ID NO:30). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellar, however, a fragment of such antigen (peptide) is presented on the surface of the cancer cells by MHC.

In certain embodiments, the encoded antigen binding domain has a binding affinity KD of 10⁻⁴ M to 10⁻⁸M.

In some embodiments, the encoded CAR molecule comprises an antigen binding domain that has a binding affinity KD of 10⁻⁴ M to 10⁻⁸M, e.g., 10⁻⁵M to 10⁻⁷ M, e.g., 10⁻⁶ M or 10⁻⁷ M, for the target antigen. In some embodiments, the antigen binding domain has a binding affinity that is at least five-fold, 10-fold, 20-fold, 30-fold, 50-fold, 100-fold or 1,000-fold less than a reference antibody, e.g., an antibody described herein. In some embodiments, the encoded antigen binding domain has a binding affinity at least 5-fold less than a reference antibody (e.g., an antibody from which the antigen binding domain is derived). In some aspects such antibody fragments are functional in that they provide a biological response that can include, but is not limited to, activation of an immune response, inhibition of signal-transduction origination from its target antigen, inhibition of kinase activity, and the like, as will be understood by a skilled artisan.

In some aspects, the antigen binding domain of the CAR is a scFv antibody fragment that is humanized compared to the murine sequence of the scFv from which it is derived.

In some aspects, the antigen binding domain of a CAR of the invention (e.g., a scFv) is encoded by a nucleic acid molecule whose sequence has been codon optimized for expression in a mammalian cell. In some aspects, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In embodiments involving immune effector cells engineered to express a CAR molecule, e.g., as described herein, it is understood that the treatment method may further include any of the steps, aspects or features described below in the section relating to Chimeric Antigen Receptors.

The cells are preferably immune effector cells. In some embodiments, the cells are T cells. In some embodiments, the cells are NK cells. In embodiments, the invention relates to a population of cells of the invention, e.g., a population of immune effector cells of the invention. In embodiments, the population of cells of the invention comprises cells of the type indicated, and may comprise other types (e.g., a population of immune effector cells, e.g., T cells, engineered to express a CAR molecule, e.g., as described herein, may include T cells engineered to express a CAR molecule as well as T cells (or other cell types) that have not been engineered to express a CAR molecule). In embodiments, the population of cells used in the methods of the invention consists essentially of cells of the type indicated. In embodiments, the population of cells of the invention is substantially free of other cell types. In embodiments, the population of cells of the invention consists of the indicated cell type.

In any of the foregoing aspects and embodiments, the cells and/or population of cells are or include immune effector cells, e.g., the population of immune effector cells includes, e.g., consists of, T cells or NK cells. In embodiments the cells are T cells, e.g., CD8+ T cells, CD4+ T cells, or a combination thereof. In embodiments the cells are NK cells.

In embodiments the cells are human cells. In embodiments, the cells are autologous, e.g., to the subject to be administered the cells. In embodiments, the cells are allogeneic, e.g., to the subject to be administered the cells.

In general, in the methods described herein, the compositions described herein may be administered in therapeutically effective amounts as described above via any of the usual and acceptable modes known in the art, either singly or in combination with one or more therapeutic agents. In particular embodiments, the compositions are administered by injection. In still particular embodiments, for in vivo administration, the compositions are administered subcutaneously to a subject in need thereof. In other embodiments, the compositions may be administered in the form of an implant at the desired site of action. The site of action may be determined by a person of skill in the art in accordance with the needs of the subject.

CAR Targets

Described herein are viral vectors to transduce immune effector cells (e.g., T cells, NK cells) that are engineered to contain one or more CARs that direct the immune effector cells to undesired cells (e.g., cancer cells). This is achieved through an antigen binding domain on the CAR that is specific for a cancer associated antigen. There are two classes of cancer associated antigens (tumor antigens) that can be targeted by the CARs of the instant invention: (1) cancer associated antigens that are expressed on the surface of cancer cells; and (2) cancer associated antigens that itself is intracellar, however, a fragment of such antigen (peptide) is presented on the surface of the cancer cells by MHC (major histocompatibility complex).

In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

CD19

An non-limiting exemplary tumor antigen is CD19. CARs that bind to CD19 are known in the art. For example, those disclosed in WO2012/079000 and WO2014/153270. Any known CD19 CAR, for example, the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Non-limiting exemplary CD19 CARs include CD19 CARs described herein or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

In some embodiments, the CD19 CAR comprises the fusion polypeptide sequence provided as SEQ ID NO: 12 in WO2012/079000, which provides an scFv fragment of murine origin that specifically binds to human CD19.

In some embodiments, the CD19 CAR comprises an amino acid sequence provided as SEQ ID NO: 12 in WO2012/079000.

In some embodiments, the CD19 CAR comprises the amino acid sequence:

(SEQ ID NO: 675)
diqmtqttsslsaslgdrvtiscrasqdiskylnwyqqkpdgtvklli
yhtsrlhsgvpsrfsgsgsgtdysltisnleqediatyfcqqgntlpy
tfgggtkleitggggsggggsggggsevklqesgpglvapsqslsvtc
tvsgvslpdygvswirqpprkglewlgviwgsettyynsalksrltii
kdnsksqvflkmnslqtddtaiyycakhyyyggsyamdywgqgtsvtv
sstttpaprpptpaptiasqplslrpeacrpaaggavhtrgldfacdi
yiwaplagtcgvlllslvitlyckrgrkkllyifkqpfmrpvqttqee
dgcscrfpeeeeggcelrvkfsrsadapaykqgqnqlynelnlgrree
ydvldkrrgrdpemggkprrknpqeglynelqkdkmaeayseigmkge
rrrgkghdglyqglstatkdtydalhmqalppr,
or a sequence substantially homologous thereto.

In some embodiments, the CD19 CAR comprises the amino acid sequence:

(SEQ ID NO: 676)
eivmtqspatlslspgeratlscrasqdiskylnwyqqkpgqaprlli
yhtsrlhsgiparfsgsgsgtdytltisslqpedfavyfcqqgntlpy
tfgqgtkleikggggsggggsggggsqvqlqesgpglykpsetlsltc
tvsgyslpdygyswirqppgkglewigviwgsettyyqsslksrvtis
kdnsknqvslklssvtaadtavyycakhyyyggsyamdywgqgtlyty
ss

In some embodiments, the CD19 CAR is a humanized CD19 CAR comprising the amino acid sequence:

(SEQ ID NO: 677)
eivmtqspatlslspgeratlscrasqdiskylnwyqqkpgqaprlli
yhtsrlhsgiparfsgsgsgtdytltisslqpedfavyfcqqgntlpy
tfgqgtkleikggggsggggsggggsqvqlqesgpglvkpsetlsltc
tvsgvslpdygvswirqppgkglewigviwgsettyyqsslksrvtis
kdnsknqvslklssvtaadtavyycakhyyyggsyamdywgqgtlvtv
sstttpaprpptpaptiasqplslrpeacrpaaggavhtrgldfacdi
yiwaplagtcgvlllslvitlyckrgrkkllyifkqpfmrpvqttqee
dgcscrfpeeeeggcelrvkfsrsadapaykqgqnqlynelnlgrree
ydvldkrrgrdpemggkprrknpqeglynelqkdkmaeayseigmkge
rrrgkghdglyqglstatkdtydalhmqalppr

In some embodiments, CD19 CARs comprise a sequence, for example, a CDR, VH, VL, scFv, or full-CAR sequence, disclosed in Table 1 below, or a sequence having at least 80%, 85%, 90%, 95%, or 99% identity thereto.

TABLE 1
Amino acid sequences of exemplary anti-CD19 molecules

| SEQ ID | | |
|---|---|---|
| NO | Region | Sequence |
| CTL019 | | |
| 678 | HCDR1 | DYGVS |
| | (Kabat) | |
| 679 | HCDR2 | VIWGSETTYYNSALKS |
| | (Kabat) | |
| 680 | HCDR3 | HYYYGGSYAMDY |
| | (Kabat) | |
| 681 | LCDRI | RASQDISKYLN |
| | (Kabat) | |
| 682 | LCDR2 | HTSRLHS |
| | (Kabat) | |
| 683 | LCDR3 | QQGNTLPYT |
| | (Kabat) | |
| 684 | CTL019 | MALPVTALLLPLALLLHAARPdiqmtqttsslsaslgdrvtiscrasqdiskyl |
| | Full | nwyqqkpdgtvklliyhtsrlhsgvpsrfsgsgsgtdysltisnleqediatyfcqqgntlpyt |
| | amino | fgggtkleitggggsggggsggggsevklqesgpglvapsqslsvtctvsgvslpdygvsw |
| | acid | irqpprkglewlgviwgsettyynsalksrltiikdnsksqvflkmnslqtddtaiyycakhy |
| | sequence | yyggsyamdywgqgtsvtvsstttpaprpptpaptiasqplslrpeacrpaaggavhtrgld |
| | | facdiyiwaplagtcgvlllslvitlyckrgrkkllyifkqpfmrpvqttqeedgcscrfpeeee |
| | | ggcelrvkfsrsadapaykqgqnqlynelnlgrreeydvldkrrgrdpemggkprrknpq |
| | | eglynelqkdkmaeayseigmkgerrrgkghdglyqglstatkdtydalhmqalppr |
| 685 | CTL019 | atggccttaccagtgaccgccttgctcctgccgctggccttgctgctccacgccgccaggccg |
| | Full | gacatccagatgacacagactacatcctccctgtctgcctctctgggagacagagtcaccatca |
| | nucleotide | gttgcagggcaagtcaggacattagtaaatatttaaattggtatcagcagaaaccagatggaact |
| | sequence | gttaaactcctgatctaccatacatcaagattacactcaggagtcccatcaaggttcagtggcag |
| | | tgggtctggaacagattattctctcaccattagcaacctggagcaagaagatattgccacttactt |
| | | ttgccaacagggtaatacgcttccgtacacgtteggaggggggaccaagctggagatcacag |
| | | gtggcggtggctcgggcggtggtgggtcgggtggcggcggatctgaggtgaaactgcagga |
| | | gtcaggacctggcctggtggcgccctcacagagcctgtccgtcacatgcactgtctcaggggt |
| | | ctcattacccgactatggtgtaagctggattcgccagcctccacgaaagggtctggagtggctg |
| | | ggagtaatatggggtagtgaaaccacatactataattcagctctcaaatccagactgaccatcat |
| | | caaggacaactccaagagccaagttttcttaaaaatgaacagtctgcaaactgatgacacagcc |
| | | atttactactgtgccaaacattattactacggtggtagctatgctatggactactggggccaagga |
| | | acctcagtcaccgtctcctcaaccacgacgccagcgccgcgaccaccaacaccggcgccca |
| | | ccatcgcgtcgcagcccctgtccctgcgcccagaggcgtgccggccagcggcggggggcg |
| | | cagtgcacacgagggggctggacttcgcctgtgatatctacatctgggcgcccttggccggga |
| | | cttgtggggtccttctcctgtcactggttatcaccctttactgcaaacggggcagaaagaaactc |
| | | ctgtatatattcaaacaaccatttatgagaccagtacaaactactcaagaggaagatggctgtag |
| | | ctgccgatttccagaagaagaagaaggaggatgtgaactgagagtgaagttcagcaggagcg |
| | | cagacgcccccgcgtacaagcagggccagaaccagctctataacgagctcaatctaggacg |
| | | aagagaggagtacgatgttttggacaagagacgtggccgggaccctgagatggggggaaag |
| | | ccgagaaggaagaaccctcaggaaggcctgtacaatgaactgcagaaagataagatggcgg |
| | | aggcctacagtgagattgggatgaaaggcgagcgccggaggggcaaggggcacgatggc |
| | | ctttaccagggtctcagtacagccaccaaggacacctacgacgcccttcacatgcaggccctg |
| | | ccccctcgc |
| 686 | CTL019 | Diqmtqttsslsaslgdrvtiscrasqdiskylnwyqqkpdgtvklliyhtsrlhsgvpsrfsg |
| | scFv | sgsgtdysltisnleqediatyfcqqgntlpytfgggtkleitggggsggggsggggsevklq |
| | domain | esgpglvapsqslsvtctvsgvslpdygvswirqpprkglewlgviwgsettyynsalksrlt |
| | | iikdnsksqvflkmnslqtddtaiyycakhyyyggsyamdywgqgtsvtvss |
| Humanized | | |
| CAR2 | | |
| 678 | HCDR1 | DYGVS |
| | (Kabat) | |
| 687 | HCDR2 | VIWGSETTYYQSSLKS |
| | (Kabat) | |
| 680 | HCDR3 | HYYYGGSYAMDY |
| | (Kabat) | |
| 681 | LCDR1 | RASQDISKYLN |
| | (Kabat) | |
| 682 | LCDR2 | HTSRLHS |
| | (Kabat) | |
| 683 | LCDR3 | QQGNTLPYT |
| | (Kabat) | |
| 676 | CAR2 | EIVMTQSPATLSLSPGERATLSCRASQDISKYLNWYQQKPG |
| | scFv | QAPRLLIYHTSRLHSGIPARFSGSGSGTDYTLTISSLQPEDFA |
| | domain - | VYFCQQGNTLPYTFGQGTKLEIKGGGGSGGGGSGGGGSQV |
| | aa | QLQESGPGLVKPSETLSLTCTVSGVSLPDYGVSWIRQPPGKG |
| | (Linker | LEWIGVIWGSETTYYQSSLKSRVTISKDNSKNQVSLKLSSVT |
| | is | AADTAVYYCAKHYYYGGSYAMDYWGQGTLVTVSS |
| | underlined) | |
| 688 | CAR2 | atggccctccctgtcaccgccctgctgcttccgctggctcttctgctccacgccgctcggcccg |
| | scEv | aaattgtgatgacccagtcacccgccactcttagcctttcacccggtgagcgcgcaaccctgtct |
| | domain - | tgcagagcctcccaagacatctcaaaataccttaattggtatcaacagaagcccggacaggctc |
| | nt | ctcgccttctgatctaccacaccagccggctccattctggaatccctgccaggttcagcggtag |
| | | cggatctgggaccgactacaccctcactatcagctcactgcagccagaggacttcgctgtctat |
| | | ttctgtcagcaagggaacaccctgccctacacctttggacagggcaccaagctcgagattaaa |
| | | ggtggaggtggcagcggaggaggtgggtccggcggtggaggaagccaggtccaactccaa |
| | | gaaagcggaccgggtcttgtgaagccatcagaaactctttcactgacttgtactgtgagcggag |
| | | tgtctctccccgattacggggtgtcttggatcagacagccaccggggaagggtctggaatgga |
| | | ttggagtgatttggggctctgagactacttactaccaatcatccctcaagtcacgcgtcaccatct |
| | | caaaggacaactctaagaatcaggtgtcactgaaactgtcatctgtgaccgcagccgacaccg |
| | | ccgtgtactattgcgctaagcattactattatggegggagctacgcaatggattactggggacag |
| | | ggtactctggtcaccgtgtccagccaccaccatcatcaccatcaccat |
| 689 | CAR 2 - | MALPVTALLLPLALLLHAARPEIVMTQSPATLSLSPGERATL |
| | Full - aa | SCRASQDISKYLNWYQQKPGQAPRLLIYHTSRLHSGIPARFS |
| | | GSGSGTDYTLTISSLQPEDFAVYFCQQGNTLPYTFGQGTKLE |
| | | IKGGGGSGGGGSGGGGSQVQLQESGPGLVKPSETLSLTCTV |
| | | SGVSLPDYGVSWIRQPPGKGLEWIGVIWGSETTYYQSSLKS |
| | | RVTISKDNSKNQVSLKLSSVTAADTAVYYCAKHYYYGGSY |
| | | AMDYWGQGTLVTVSSTTTPAPRPPTPAPTIASQPLSLRPEAC |
| | | RPAAGGAVHTRGLDFACDIYIWAPLAGTCGVLLLSLVITLY |
| | | CKRGRKKLLYIFKQPFMRPVQTTQEEDGCSCRFPEEEEGGC |
| | | ELRVKFSRSADAPAYKQGQNQLYNELNLGRREEYDVLDKR |
| | | RGRDPEMGGKPRRKNPQEGLYNELQKDKMAEAYSEIGMK |
| | | GERRRGKGHDGLYQGLSTATKDTYDALHMQALPPR |
| 690 | CAR 2 - | atggccctccctgtcaccgccctgctgcttccgctggctcttctgctccacgccgctcggcccg |
| | Full - nt | aaattgtgatgacccagtcacccgccactcttagcctttcacccggtgagcgcgcaaccctgtct |
| | | tgcagagcctcccaagacatctcaaaataccttaattggtatcaacagaagcccggacaggctc |
| | | ctcgccttctgatctaccacaccagccggctccattctggaatccctgccaggttcagcggtag |
| | | cggatctgggaccgactacaccctcactatcagctcactgcagccagaggacttcgctgtctat |
| | | ttctgtcagcaagggaacaccctgccctacacctttggacagggcaccaagctcgagattaaa |
| | | ggtggaggtggcagcggaggaggtgggtccggcggtggaggaagccaggtccaactccaa |
| | | gaaagcggaccgggtcttgtgaagccatcagaaactctttcactgacttgtactgtgagcggag |
| | | tgtctctccccgattacggggtgtcttggatcagacagccaccggggaagggtctggaatgga |
| | | ttggagtgatttggggctctgagactacttactaccaatcatccctcaagtcacgcgtcaccatct |
| | | caaaggacaactctaagaatcaggtgtcactgaaactgtcatctgtgaccgcagccgacaccg |
| | | ccgtgtactattgcgctaagcattactattatggcgggagctacgcaatggattactggggacag |
| | | ggtactctggtcaccgtgtccagcaccactaccccagcaccgaggccacccaccccggctcc |
| | | taccatcgcctcccagcctctgtccctgcgtccggaggcatgtagacccgcagctggtggggc |
| | | cgtgcatacccggggtcttgacttcgcctgcgatatctacatttgggcccctctggctggtacttg |
| | | cggggtcctgctgctttcactcgtgatcactctttactgtaagcgcggtcggaagaagctgctgt |
| | | acatctttaagcaacccttcatgaggcctgtgcagactactcaagaggaggacggctgttcatg |
| | | ccggttcccagaggaggaggaaggcggctgcgaactgcgcgtgaaattcagccgcagcgc |
| | | agatgctccagcctacaagcaggggcagaaccagctctacaacgaactcaatcttggtcgga |
| | | gagaggagtacgacgtgctggacaagcggagaggacgggacccagaaatgggcgggaag |
| | | ccgcgcagaaagaatccccaagagggcctgtacaacgagctccaaaaggataagatggcag |
| | | aagcctatagcgagattggtatgaaaggggaacgcagaagaggcaaaggccacgacggact |
| | | gtaccagggactcagcaccgccaccaaggacacctatgacgctcttcacatgcaggccctgc |
| | | cgcctcgg |

BCMA

A non-limiting exemplary tumor antigen is BCMA. CARs that bind to BCMA are known in the art. For example, those disclosed WO2016/014565 or WO2019/241426. Any known BCMA CAR, for example, the BCMA antigen binding domain of any known BCMA CAR, in the art can be used in accordance with the present disclosure. For example, BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1, disclosed in WO2016/014565.

In some embodiments, the BCMA CAR comprises one or more CDRs, VH, VL, scFv, or full-length sequences of BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1 disclosed in WO2016/014565, or a sequence substantially (for example, 95-99%) identical thereto.

In some embodiments, a BCMA CAR comprises a sequence, for example, a CDR, VH, VL, scFv, or full-CAR sequence, disclosed in Tables 2-14, or a sequence having at least 80%, 85%, 90%, 95%, or 99% identity thereto.

TABLE 2
Amino acid and nucleic acid sequences of exemplary
PALLAS-derived anti-BCMA molecules

| SEQ | Name/ | |
|---|---|---|
| ID NO | Description | Sequence |
| R1B6 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWVPYDVSWYFDY |
| NO: 46 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWVPYDVSWYFDY |
| NO: 46 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWVPYDVSWYFDY |
| NO: 51 | (IMGT) | |
| SEQ ID | VH | EVQLLESGGGLVQPGGSLRLSCAASGFTFSSYAMSWVRQAP |
| NO: 52 | | GKGLEWVSAISGSGGSTYYADSVKGRFTISRDNSKNTLYLQ |
| | | MNSLRAEDTAVYYCARREWVPYDVSWYFDYWGQGTLVT |
| | | VSS |
| SEQ ID | DNA VH | GAAGTGCAGTTGCTGGAGTCAGGCGGAGGACTGGTGCAG |
| NO: 53 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGGTGCCCTACGAT |
| | | GTCAGCTGGTACTTCGACTACTGGGGACAGGGCACTCTC |
| | | GTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGK |
| NO: 61 | | APKLLIYAASSLQSGVPSRFSGSGSGTDFTLTISSLQPEDFAT |
| | | YYCQQSYSTPLTFGQGTKVEIK |
| SEQ ID | DNA VL | GACATTCAAATGACTCAGTCCCCGTCCTCCCTCTCCGCCT |
| NO: 62 | | CCGTGGGAGATCGCGTCACGATCACGTGCAGGGCCAGCC |
| | | AGAGCATCTCCAGCTACCTGAACTGGTACCAGCAGAAGC |
| | | CAGGGAAGGCACCGAAGCTCCTGATCTACGCCGCTAGCT |
| | | CGCTGCAGTCCGGCGTCCCTTCACGGTTCTCGGGATCGGG |
| | | CTCAGGCACCGACTTCACCCTGACCATTAGCAGCCTGCAG |
| | | CCGGAGGACTTCGCGACATACTACTGTCAGCAGTCATACT |
| | | CCACCCCTCTGACCTTCGGCCAAGGGACCAAAGTGGAGA |
| | | TCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAP |
| NO: 64 | linker-VL) | GKGLEWVSAISGSGGSTYYADSVKGRFTISRDNSKNTLYLQ |
| | | MNSLRAEDTAVYYCARREWVPYDVSWYFDYWGQGTLVT |
| | | VSSGGGGSGGGGSGGGGSGGGGSDIQMTQSPSSLSASVGDR |
| | | VTITCRASQSISSYLNWYQQKPGKAPKLLIYAASSLQSGVPS |
| | | RFSGSGSGTDFTLTISSLQPEDFATYYCQQSYSTPLTFGQGTK |
| | | VEIK |
| SEQ ID | DNA scFv | GAAGTGCAGTTGCTGGAGTCAGGCGGAGGACTGGTGCAG |
| NO: 65 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGGTGCCCTACGAT |
| | | GTCAGCTGGTACTTCGACTACTGGGGACAGGGCACTCTC |
| | | GTGACTGTGTCCTCCGGTGGTGGTGGATCGGGGGGTGGT |
| | | GGTTCGGGCGGAGGAGGATCTGGAGGAGGAGGGTCGGA |
| | | CATTCAAATGACTCAGTCCCCGTCCTCCCTCTCCGCCTCC |
| | | GTGGGAGATCGCGTCACGATCACGTGCAGGGCCAGCCAG |
| | | AGCATCTCCAGCTACCTGAACTGGTACCAGCAGAAGCCA |
| | | GGGAAGGCACCGAAGCTCCTGATCTACGCCGCTAGCTCG |
| | | CTGCAGTCCGGCGTCCCTTCACGGTTCTCGGGATCGGGCT |
| | | CAGGCACCGACTTCACCCTGACCATTAGCAGCCTGCAGC |
| | | CGGAGGACTTCGCGACATACTACTGTCAGCAGTCATACTC |
| | | CACCCCTCTGACCTTCGGCCAAGGGACCAAAGTGGAGAT |
| | | CAAG |
| SEQ ID | Full CAR | EVQLLESGGGLVQPGGSLRLSCAASGFTFSSYAMSWVRQAP |
| NO: 66 | amino acid | GKGLEWVSAISGSGGSTYYADSVKGRFTISRDNSKNTLYLQ |
| | sequence | MNSLRAEDTAVYYCARREWVPYDVSWYFDYWGQGTLVT |
| | | VSSGGGGSGGGGSGGGGSGGGGSDIQMTQSPSSLSASVGDR |
| | | VTITCRASQSISSYLNWYQQKPGKAPKLLIYAASSLQSGVPS |
| | | RFSGSGSGTDFTLTISSLQPEDFATYYCQQSYSTPLTFGQGTK |
| | | VEIKTTTPAPRPPTPAPTIASQPLSLRPEACRPAAGGAVHTRG |
| | | LDFACDIYIWAPLAGTCGVLLLSLVITLYCKRGRKKLLYIFK |
| | | QPFMRPVQTTQEEDGCSCRFPEEEEGGCELRVKFSRSADAP |
| | | AYQQGQNQLYNELNLGRREEYDVLDKRRGRDPEMGGKPR |
| | | RKNPQEGLYNELQKDKMAEAYSEIGMKGERRRGKGHDGL |
| | | YQGLSTATKDTYDALHMQALPPR |
| SEQ ID | Full CAR | GAAGTGCAGTTGCTGGAGTCAGGCGGAGGACTGGTGCAG |
| NO: 67 | DNA | CCCGGAGGATCGCTTCGCTTGAGCTGCGCAGCCTCAGGCT |
| | sequence | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGGTGCCCTACGAT |
| | | GTCAGCTGGTACTTCGACTACTGGGGACAGGGCACTCTC |
| | | GTGACTGTGTCCTCCGGTGGTGGTGGATCGGGGGGTGGT |
| | | GGTTCGGGCGGAGGAGGATCTGGAGGAGGAGGGTCGGA |
| | | CATTCAAATGACTCAGTCCCCGTCCTCCCTCTCCGCCTCC |
| | | GTGGGAGATCGCGTCACGATCACGTGCAGGGCCAGCCAG |
| | | AGCATCTCCAGCTACCTGAACTGGTACCAGCAGAAGCCA |
| | | GGGAAGGCACCGAAGCTCCTGATCTACGCCGCTAGCTCG |
| | | CTGCAGTCCGGCGTCCCTTCACGGTTCTCGGGATCGGGCT |
| | | CAGGCACCGACTTCACCCTGACCATTAGCAGCCTGCAGC |
| | | CGGAGGACTTCGCGACATACTACTGTCAGCAGTCATACTC |
| | | CACCCCTCTGACCTTCGGCCAAGGGACCAAAGTGGAGAT |
| | | CAAGACCACTACCCCAGCACCGAGGCCACCCACCCCGGC |
| | | TCCTACCATCGCCTCCCAGCCTCTGTCCCTGCGTCCGGAG |
| | | GCATGTAGACCCGCAGCTGGTGGGGCCGTGCATACCCGG |
| | | GGTCTTGACTTCGCCTGCGATATCTACATTTGGGCCCCTC |
| | | TGGCTGGTACTTGCGGGGTCCTGCTGCTTTCACTCGTGAT |
| | | CACTCTTTACTGTAAGCGCGGTCGGAAGAAGCTGCTGTAC |
| | | ATCTTTAAGCAACCCTTCATGAGGCCTGTGCAGACTACTC |
| | | AAGAGGAGGACGGCTGTTCATGCCGGTTCCCAGAGGAGG |
| | | AGGAAGGCGGCTGCGAACTGCGCGTGAAATTCAGCCGCA |
| | | GCGCAGATGCTCCAGCCTACCAGCAGGGGCAGAACCAGC |
| | | TCTACAACGAACTCAATCTTGGTCGGAGAGAGGAGTACG |
| | | ACGTGCTGGACAAGCGGAGAGGACGGGACCCAGAAATG |
| | | GGCGGGAAGCCGCGCAGAAAGAATCCCCAAGAGGGCCT |
| | | GTACAACGAGCTCCAAAAGGATAAGATGGCAGAAGCCTA |
| | | TAGCGAGATTGGTATGAAAGGGGAACGCAGAAGAGGCA |
| | | AAGGCCACGACGGACTGTACCAGGGACTCAGCACCGCCA |
| | | CCAAGGACACCTATGACGCTCTTCACATGCAGGCCCTGCC |
| | | GCCTCGG |
| R1F2 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWYDDWYLDY |
| NO: 68 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWYDDWYLDY |
| NO: 68 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWYDDWYLDY |
| NO: 69 | (IMGT) | |
| SEQ ID | VH | EVQLLESGGGLVQPGGSLRLSCAASGFTFSSYAMSWVRQAP |
| NO: 70 | | GKGLEWVSAISGSGGSTYYADSVKGRFTISRDNSKNTLYLQ |
| | | MNSLRAEDTAVYYCARREWWYDDWYLDYWGQGTLVTVS |
| | | S |
| SEQ ID | DNA VH | GAAGTGCAGTTGCTGGAGTCAGGCGGAGGACTGGTGCAG |
| NO: 71 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGTACGACGAT |
| | | TGGTACCTGGACTACTGGGGACAGGGCACTCTCGTGACT |
| | | GTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGK |
| NO: 61 | | APKLLIYAASSLQSGVPSRFSGSGSGTDFTLTISSLQPEDFAT |
| | | YYCQQSYSTPLTFGQGTKVEIK |
| SEQ ID | DNA VL | GACATTCAAATGACTCAGTCCCCGTCCTCCCTCTCCGCCT |
| NO: 62 | | CCGTGGGAGATCGCGTCACGATCACGTGCAGGGCCAGCC |
| | | AGAGCATCTCCAGCTACCTGAACTGGTACCAGCAGAAGC |
| | | CAGGGAAGGCACCGAAGCTCCTGATCTACGCCGCTAGCT |
| | | CGCTGCAGTCCGGCGTCCCTTCACGGTTCTCGGGATCGGG |
| | | CTCAGGCACCGACTTCACCCTGACCATTAGCAGCCTGCAG |
| | | CCGGAGGACTTCGCGACATACTACTGTCAGCAGTCATACT |
| | | CCACCCCTCTGACCTTCGGCCAAGGGACCAAAGTGGAGA |
| | | TCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAP |
| NO: 72 | linker-VL) | GKGLEWVSAISGSGGSTYYADSVKGRFTISRDNSKNTLYLQ |
| | | MNSLRAEDTAVYYCARREWWYDDWYLDYWGQGTLVTVS |
| | | SGGGGSGGGGSGGGGSGGGGSDIQMTQSPSSLSASVGDRVT |
| | | ITCRASQSISSYLNWYQQKPGKAPKLLIYAASSLQSGVPSRF |
| | | SGSGSGTDFTLTISSLQPEDFATYYCQQSYSTPLTFGQGTKV |
| | | EIK |
| SEQ ID | DNA scFv | GAAGTGCAGTTGCTGGAGTCAGGCGGAGGACTGGTGCAG |
| NO: 73 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGTACGACGAT |
| | | TGGTACCTGGACTACTGGGGACAGGGCACTCTCGTGACT |
| | | GTGTCCTCCGGTGGTGGTGGATCGGGGGGTGGTGGTTCG |
| | | GGCGGAGGAGGATCTGGAGGAGGAGGGTCGGACATTCA |
| | | AATGACTCAGTCCCCGTCCTCCCTCTCCGCCTCCGTGGGA |
| | | GATCGCGTCACGATCACGTGCAGGGCCAGCCAGAGCATC |
| | | TCCAGCTACCTGAACTGGTACCAGCAGAAGCCAGGGAAG |
| | | GCACCGAAGCTCCTGATCTACGCCGCTAGCTCGCTGCAGT |
| | | CCGGCGTCCCTTCACGGTTCTCGGGATCGGGCTCAGGCAC |
| | | CGACTTCACCCTGACCATTAGCAGCCTGCAGCCGGAGGA |
| | | CTTCGCGACATACTACTGTCAGCAGTCATACTCCACCCCT |
| | | CTGACCTTCGGCCAAGGGACCAAAGTGGAGATCAAG |
| SEQ ID | Full CAR | EVQLLESGGGLVQPGGSLRLSCAASGFTFSSYAMSWVRQAP |
| NO: 74 | amino acid | GKGLEWVSAISGSGGSTYYADSVKGRFTISRDNSKNTLYLQ |
| | sequence | MNSLRAEDTAVYYCARREWWYDDWYLDYWGQGTLVTVS |
| | | SGGGGSGGGGSGGGGSGGGGSDIQMTQSPSSLSASVGDRVT |
| | | ITCRASQSISSYLNWYQQKPGKAPKLLIYAASSLQSGVPSRF |
| | | SGSGSGTDFTLTISSLQPEDFATYYCQQSYSTPLTFGQGTKV |
| | | EIKTTTPAPRPPTPAPTIASQPLSLRPEACRPAAGGAVHTRGL |
| | | DFACDIYIWAPLAGTCGVLLLSLVITLYCKRGRKKLLYIFKQ |
| | | PFMRPVQTTQEEDGCSCRFPEEEEGGCELRVKFSRSADAPA |
| | | YQQGQNQLYNELNLGRREEYDVLDKRRGRDPEMGGKPRR |
| | | KNPQEGLYNELQKDKMAEAYSEIGMKGERRRGKGHDGLY |
| | | QGLSTATKDTYDALHMQALPPR |
| SEQ ID | Full CAR | GAAGTGCAGTTGCTGGAGTCAGGCGGAGGACTGGTGCAG |
| NO: 75 | DNA | CCCGGAGGATCGCTTCGCTTGAGCTGCGCAGCCTCAGGCT |
| | sequence | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGTACGACGAT |
| | | TGGTACCTGGACTACTGGGGACAGGGCACTCTCGTGACT |
| | | GTGTCCTCCGGTGGTGGTGGATCGGGGGGTGGTGGTTCG |
| | | GGCGGAGGAGGATCTGGAGGAGGAGGGTCGGACATTCA |
| | | AATGACTCAGTCCCCGTCCTCCCTCTCCGCCTCCGTGGGA |
| | | GATCGCGTCACGATCACGTGCAGGGCCAGCCAGAGCATC |
| | | TCCAGCTACCTGAACTGGTACCAGCAGAAGCCAGGGAAG |
| | | GCACCGAAGCTCCTGATCTACGCCGCTAGCTCGCTGCAGT |
| | | CCGGCGTCCCTTCACGGTTCTCGGGATCGGGCTCAGGCAC |
| | | CGACTTCACCCTGACCATTAGCAGCCTGCAGCCGGAGGA |
| | | CTTCGCGACATACTACTGTCAGCAGTCATACTCCACCCCT |
| | | CTGACCTTCGGCCAAGGGACCAAAGTGGAGATCAAGACC |
| | | ACTACCCCAGCACCGAGGCCACCCACCCCGGCTCCTACC |
| | | ATCGCCTCCCAGCCTCTGTCCCTGCGTCCGGAGGCATGTA |
| | | GACCCGCAGCTGGTGGGGCCGTGCATACCCGGGGTCTTG |
| | | ACTTCGCCTGCGATATCTACATTTGGGCCCCTCTGGCTGG |
| | | TACTTGCGGGGTCCTGCTGCTTTCACTCGTGATCACTCTTT |
| | | ACTGTAAGCGCGGTCGGAAGAAGCTGCTGTACATCTTTA |
| | | AGCAACCCTTCATGAGGCCTGTGCAGACTACTCAAGAGG |
| | | AGGACGGCTGTTCATGCCGGTTCCCAGAGGAGGAGGAAG |
| | | GCGGCTGCGAACTGCGCGTGAAATTCAGCCGCAGCGCAG |
| | | ATGCTCCAGCCTACCAGCAGGGGCAGAACCAGCTCTACA |
| | | ACGAACTCAATCTTGGTCGGAGAGAGGAGTACGACGTGC |
| | | TGGACAAGCGGAGAGGACGGGACCCAGAAATGGGCGGG |
| | | AAGCCGCGCAGAAAGAATCCCCAAGAGGGCCTGTACAAC |
| | | GAGCTCCAAAAGGATAAGATGGCAGAAGCCTATAGCGAG |
| | | ATTGGTATGAAAGGGGAACGCAGAAGAGGCAAAGGCCA |
| | | CGACGGACTGTACCAGGGACTCAGCACCGCCACCAAGGA |
| | | CACCTATGACGCTCTTCACATGCAGGCCCTGCCGCCTCGG |
| R1G5 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWGESWLFDY |
| NO: 76 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWGESWLFDY |
| NO: 76 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWGESWLFDY |
| NO: 77 | (IMGT) | |
| SEQ ID | VH | EVQLLESGGGLVQPGGSLRLSCAASGFTFSSYAMSWVRQAP |
| NO: 78 | | GKGLEWVSAISGSGGSTYYADSVKGRFTISRDNSKNTLYLQ |
| | | MNSLRAEDTAVYYCARREWWGESWLFDYWGQGTLVTVSS |
| SEQ ID | DNA VH | GAAGTGCAGTTGCTGGAGTCAGGCGGAGGACTGGTGCAG |
| NO: 79 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGGGAGAAAGC |
| | | TGGCTGTTCGACTACTGGGGACAGGGCACTCTCGTGACTG |
| | | TGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGK |
| NO: 61 | | APKLLIYAASSLQSGVPSRFSGSGSGTDFTLTISSLQPEDFAT |
| | | YYCQQSYSTPLTFGQGTKVEIK |
| SEQ ID | DNA VL | GACATTCAAATGACTCAGTCCCCGTCCTCCCTCTCCGCCT |
| NO: 62 | | CCGTGGGAGATCGCGTCACGATCACGTGCAGGGCCAGCC |
| | | AGAGCATCTCCAGCTACCTGAACTGGTACCAGCAGAAGC |
| | | CAGGGAAGGCACCGAAGCTCCTGATCTACGCCGCTAGCT |
| | | CGCTGCAGTCCGGCGTCCCTTCACGGTTCTCGGGATCGGG |
| | | CTCAGGCACCGACTTCACCCTGACCATTAGCAGCCTGCAG |
| | | CCGGAGGACTTCGCGACATACTACTGTCAGCAGTCATACT |
| | | CCACCCCTCTGACCTTCGGCCAAGGGACCAAAGTGGAGA |
| | | TCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAP |
| NO: 80 | linker-VL) | GKGLEWVSAISGSGGSTYYADSVKGRFTISRDNSKNTLYLQ |
| | | MNSLRAEDTAVYYCARREWWGESWLFDYWGQGTLVTVSS |
| | | GGGGSGGGGSGGGGSGGGGSDIQMTQSPSSLSASVGDRVTI |
| | | TCRASQSISSYLNWYQQKPGKAPKLLIYAASSLQSGVPSRFS |
| | | GSGSGTDFTLTISSLQPEDFATYYCQQSYSTPLTFGQGTKVEI |
| | | K |
| SEQ ID | DNA scFv | GAAGTGCAGTTGCTGGAGTCAGGCGGAGGACTGGTGCAG |
| NO: 81 | | CCCGGAGGATCGCTTCGCTTGAGCTGCGCAGCCTCAGGCT |
| | | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGGGAGAAAGC |
| | | TGGCTGTTCGACTACTGGGGACAGGGCACTCTCGTGACTG |
| | | TGTCCTCCGGTGGTGGTGGATCGGGGGGTGGTGGTTCGG |
| | | GCGGAGGAGGATCTGGAGGAGGAGGGTCGGACATTCAA |
| | | ATGACTCAGTCCCCGTCCTCCCTCTCCGCCTCCGTGGGAG |
| | | ATCGCGTCACGATCACGTGCAGGGCCAGCCAGAGCATCT |
| | | CCAGCTACCTGAACTGGTACCAGCAGAAGCCAGGGAAGG |
| | | CACCGAAGCTCCTGATCTACGCCGCTAGCTCGCTGCAGTC |
| | | CGGCGTCCCTTCACGGTTCTCGGGATCGGGCTCAGGCACC |
| | | GACTTCACCCTGACCATTAGCAGCCTGCAGCCGGAGGAC |
| | | TTCGCGACATACTACTGTCAGCAGTCATACTCCACCCCTC |
| | | TGACCTTCGGCCAAGGGACCAAAGTGGAGATCAAG |
| SEQ ID | Full CAR | EVQLLESGGGLVQPGGSLRLSCAASGFTFSSYAMSWVRQAP |
| NO: 82 | amino acid | GKGLEWVSAISGSGGSTYYADSVKGRFTISRDNSKNTLYLQ |
| | sequence | MNSLRAEDTAVYYCARREWWGESWLFDYWGQGTLVTVSS |
| | | GGGGSGGGGSGGGGSGGGGSDIQMTQSPSSLSASVGDRVTI |
| | | TCRASQSISSYLNWYQQKPGKAPKLLIYAASSLQSGVPSRFS |
| | | GSGSGTDFTLTISSLQPEDFATYYCQQSYSTPLTFGQGTKVEI |
| | | KTTTPAPRPPTPAPTIASQPLSLRPEACRPAAGGAVHTRGLD |
| | | FACDIYIWAPLAGTCGVLLLSLVITLYCKRGRKKLLYIFKQP |
| | | FMRPVQTTQEEDGCSCRFPEEEEGGCELRVKFSRSADAPAY |
| | | QQGQNQLYNELNLGRREEYDVLDKRRGRDPEMGGKPRRK |
| | | NPQEGLYNELQKDKMAEAYSEIGMKGERRRGKGHDGLYQ |
| | | GLSTATKDTYDALHMQALPPR |
| SEQ ID | Full CAR | GAAGTGCAGTTGCTGGAGTCAGGCGGAGGACTGGTGCAG |
| NO: 83 | DNA | CCCGGAGGATCGCTTCGCTTGAGCTGCGCAGCCTCAGGCT |
| | sequence | TTACCTTCTCCTCCTACGCCATGTCCTGGGTCAGACAGGC |
| | | TCCCGGGAAGGGACTGGAATGGGTGTCCGCCATTAGCGG |
| | | TTCCGGCGGAAGCACTTACTATGCCGACTCTGTGAAGGG |
| | | CCGCTTCACTATCTCCCGGGACAACTCCAAGAACACCCTG |
| | | TATCTCCAAATGAATTCCCTGAGGGCCGAAGATACCGCG |
| | | GTGTACTACTGCGCTAGACGGGAGTGGTGGGGAGAAAGC |
| | | TGGCTGTTCGACTACTGGGGACAGGGCACTCTCGTGACTG |
| | | TGTCCTCCGGTGGTGGTGGATCGGGGGGTGGTGGTTCGG |
| | | GCGGAGGAGGATCTGGAGGAGGAGGGTCGGACATTCAA |
| | | ATGACTCAGTCCCCGTCCTCCCTCTCCGCCTCCGTGGGAG |
| | | ATCGCGTCACGATCACGTGCAGGGCCAGCCAGAGCATCT |
| | | CCAGCTACCTGAACTGGTACCAGCAGAAGCCAGGGAAGG |
| | | CACCGAAGCTCCTGATCTACGCCGCTAGCTCGCTGCAGTC |
| | | CGGCGTCCCTTCACGGTTCTCGGGATCGGGCTCAGGCACC |
| | | GACTTCACCCTGACCATTAGCAGCCTGCAGCCGGAGGAC |
| | | TTCGCGACATACTACTGTCAGCAGTCATACTCCACCCCTC |
| | | TGACCTTCGGCCAAGGGACCAAAGTGGAGATCAAGACCA |
| | | CTACCCCAGCACCGAGGCCACCCACCCCGGCTCCTACCA |
| | | TCGCCTCCCAGCCTCTGTCCCTGCGTCCGGAGGCATGTAG |
| | | ACCCGCAGCTGGTGGGGCCGTGCATACCCGGGGTCTTGA |
| | | CTTCGCCTGCGATATCTACATTTGGGCCCCTCTGGCTGGT |
| | | ACTTGCGGGGTCCTGCTGCTTTCACTCGTGATCACTCTTT |
| | | ACTGTAAGCGCGGTCGGAAGAAGCTGCTGTACATCTTTA |
| | | AGCAACCCTTCATGAGGCCTGTGCAGACTACTCAAGAGG |
| | | AGGACGGCTGTTCATGCCGGTTCCCAGAGGAGGAGGAAG |
| | | GCGGCTGCGAACTGCGCGTGAAATTCAGCCGCAGCGCAG |
| | | ATGCTCCAGCCTACCAGCAGGGGCAGAACCAGCTCTACA |
| | | ACGAACTCAATCTTGGTCGGAGAGAGGAGTACGACGTGC |
| | | TGGACAAGCGGAGAGGACGGGACCCAGAAATGGGCGGG |
| | | AAGCCGCGCAGAAAGAATCCCCAAGAGGGCCTGTACAAC |
| | | GAGCTCCAAAAGGATAAGATGGCAGAAGCCTATAGCGAG |
| | | ATTGGTATGAAAGGGGAACGCAGAAGAGGCAAAGGCCA |
| | | CGACGGACTGTACCAGGGACTCAGCACCGCCACCAAGGA |
| | | CACCTATGACGCTCTTCACATGCAGGCCCTGCCGCCTCGG |

TABLE 3
Kabat CDRs of exemplary PALLAS-derived anti-BCMA molecules

| Kabat | HCDR1 | HCDR2 | HCDR3 | LCDR1 | LCDR2 | LCDR3 |
|---|---|---|---|---|---|---|
| R1B6 | SYAMS | AISGSGGSTY | REWVPYDV | RASQSISS | AASSL | QQSYST |
| | (SEQ ID | YADSVKG | SWYFDY | YLN (SEQ | QS | PLT |
| | NO: 44) | (SEQ ID NO: | (SEQ ID NO: | ID NO: 54) | (SEQ | (SEQ ID |
| | | 45) | 46) | | ID NO: | NO: 56) |
| | | | | | 55) | |
| R1F2 | SYAMS | AISGSGGSTY | REWWYDD | RASQSISS | AASSL | QQSYST |
| | (SEQ ID | YADSVKG | WYLDY | YLN (SEQ | QS | PLT |
| | NO: 44) | (SEQ ID NO: | (SEQ ID NO: | ID NO: 54) | (SEQ | (SEQ ID |
| | | 45) | 68) | | ID NO: | NO: 56) |
| | | | | | 55) | |
| RIG5 | SYAMS | AISGSGGSTY | REWWGES | RASQSISS | AASSL | QQSYST |
| | (SEQ ID | YADSVKG | WLFDY | YLN (SEQ | QS | PLT |
| | NO: 44) | (SEQ ID NO: | (SEQ ID NO: | ID NO: 54) | (SEQ | (SEQ ID |
| | | 45) | 76) | | ID NO: | NO: 56) |
| | | | | | 55) | |
| Consensus | SYAMS | AISGSGGSTY | REWX₁X₂X₃ | RASQSISS | AASSL | QQSYST |
| | (SEQ ID | YADSVKG | X₄X₅X₆WX₇ | YLN (SEQ | QS | PLT |
| | NO: 44) | (SEQ ID NO: | X₈DY, | ID NO: 54) | (SEQ | (SEQ ID |
| | | 45) | wherein X₁ is | | ID NO: | NO: 56) |
| | | | absent or V; | | 55) | |
| | | | X₂ is absent | | | |
| | | | or P; X₃ is W | | | |
| | | | or Y; X₄ is G, | | | |
| | | | Y, or D; X₅ is | | | |
| | | | E, D, or V; X₆ | | | |
| | | | is S or D; X₇ | | | |
| | | | is L or Y; and | | | |
| | | | X₈ is F or L | | | |
| | | | (SEQ ID NO: | | | |
| | | | 84) | | | |

TABLE 4
Chothia CDRs of exemplary PALLAS-derived anti-BCMA molecules

| Chothia | HCDR1 | HCDR2 | HCDR3 | LCDR1 | LCDR2 | LCDR3 |
|---|---|---|---|---|---|---|
| R1B6 | GFTFSSY | SGSGGS | REWVPYDV | SQSISSY | AAS | SYSTPL |
| | (SEQ ID | (SEQ ID NO: | SWYFDY | (SEQ ID | (SEQ | (SEQ ID |
| | NO: 47) | 48) | (SEQ ID NO: | NO: 57) | ID NO: | NO: 59) |
| | | | 46) | | 58) | |
| R1F2 | GFTFSSY | SGSGGS | REWWYDD | SQSISSY | AAS | SYSTPL |
| | (SEQ ID | (SEQ ID NO: | WYLDY | (SEQ ID | (SEQ | (SEQ ID |
| | NO: 47) | 48) | (SEQ ID NO: | NO: 57) | ID NO: | NO: 59) |
| | | | 68) | | 58) | |
| RIG5 | GFTFSSY | SGSGGS | REWWGES | SQSISSY | AAS | SYSTPL |
| | (SEQ ID | (SEQ ID NO: | WLFDY | (SEQ ID | (SEQ | (SEQ ID |
| | NO: 47) | 48) | (SEQ ID NO: | NO: 57) | ID NO: | NO: 59) |
| | | | 76) | | 58) | |
| Consensus | GFTFSSY | SGSGGS | REWX₁X₂X₃ | SQSISSY | AAS | SYSTPL |
| | (SEQ ID | (SEQ ID NO: | X₄X₅X₆WX₇ | (SEQ ID | (SEQ | (SEQ ID |
| | NO: 47) | 48) | X₈DY, | NO: 57) | ID NO: | NO: 59) |
| | | | wherein X₁ is | | 58) | |
| | | | absent or V; | | | |
| | | | X₂ is absent | | | |
| | | | or P; X₃ is W | | | |
| | | | or Y; X₄ is G, | | | |
| | | | Y, or D; X₅ is | | | |
| | | | E, D, or V; X₆ | | | |
| | | | is S or D; X₇ | | | |
| | | | is L or Y; and | | | |
| | | | X₈ is F or L | | | |
| | | | (SEQ ID NO: | | | |
| | | | 84) | | | |

TABLE 5
IMGT CDRs of exemplary PALLAS-derived anti-BCMA molecules

| IMGT | HCDR1 | HCDR2 | HCDR3 | LCDR1 | LCDR2 | LCDR3 |
|---|---|---|---|---|---|---|
| R1B6 | GFTFSSY | ISGSGGST | ARREWVPY | QSISSY | AAS | QQSYST |
| | A (SEQ ID | (SEQ ID NO: | DVSWYFDY | (SEQ ID | (SEQ | PLT |
| | NO: 49) | 50) | (SEQ ID NO: | NO: 60) | ID NO: | (SEQ ID |
| | | | 51) | | 58) | NO: 56) |
| R1F2 | GFTFSSY | ISGSGGST | ARREWWY | QSISSY | AAS | QQSYST |
| | A (SEQ ID | (SEQ ID NO: | DDWYLDY | (SEQ ID | (SEQ | PLT |
| | NO: 49) | 50) | (SEQ ID NO: | NO: 60) | ID NO: | (SEQ ID |
| | | | 69) | | 58) | NO: 56) |
| RIG5 | GFTFSSY | ISGSGGST | ARREWWG | QSISSY | AAS | QQSYST |
| | A (SEQ ID | (SEQ ID NO: | ESWLFDY | (SEQ ID | (SEQ | PLT |
| | NO: 49) | 50) | (SEQ ID NO: | NO: 60) | ID NO: | (SEQ ID |
| | | | 77) | | 58) | NO: 56) |
| Consensus | GFTFSSY | ISGSGGST | ARREWX₁X₂ | QSISSY | AAS | QQSYST |
| | A (SEQ ID | (SEQ ID NO: | X₃X₄X₅X₆W | (SEQ ID | (SEQ | PLT |
| | NO: 49) | 50) | X₇X₈DY, | NO: 60) | ID NO: | (SEQ ID |
| | | | wherein X₁ is | | 58) | NO: 56) |
| | | | absent or V; | | | |
| | | | X₂ is absent | | | |
| | | | or P; X₃ is W | | | |
| | | | or Y; X₄ is G, | | | |
| | | | Y, or D; X₅ is | | | |
| | | | E, D, or V; X₆ | | | |
| | | | is S or D; X₇ | | | |
| | | | is L or Y; and | | | |
| | | | X₈ is F or L | | | |
| | | | (SEQ ID NO: | | | |
| | | | 85) | | | |

TABLE 6
Amino acid and nucleic acid sequences of exemplary B
cell-derived anti-BCMA molecules

| SEQ | Name/ | |
|---|---|---|
| ID NO | Description | Sequence |
| P161 | | |
| SEQ ID | HCDR1 | SYGMH |
| NO: 86 | (Kabat) | |
| SEQ ID | HCDR2 | VISYDGSNKYYADSVKG |
| NO: 87 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYDGSN |
| NO: 89 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYDGSNK |
| NO: 91 | (IMGT) | |
| SEQ ID | HCDR3 | GGSGYALHDDYYGLDV |
| NO: 92 | (IMGT) | |
| SEQ ID | VH | QVQLQESGGGVVQPGRSLRLSCAASGFTFSSYGMHWVRQAP |
| NO: 93 | | GKGLEWVAVISYDGSNKYYADSVKGRFTISRDNSKNTLYLQ |
| | | MNSLRAEDTAVYYCGGSGYALHDDYYGLDVWGQGTLVTV |
| | | SS |
| SEQ ID | DNA VH | CAAGTGCAGCTGCAGGAATCCGGTGGCGGAGTCGTGCAG |
| NO: 94 | | CCTGGAAGGAGCCTGAGACTCTCATGCGCCGCGTCAGGGT |
| | | TCACCTTTTCCTCCTACGGGATGCATTGGGTCAGACAGGC |
| | | CCCCGGAAAGGGACTCGAATGGGTGGCTGTGATCAGCTAC |
| | | GACGGCTCCAACAAGTACTACGCCGACTCCGTGAAAGGCC |
| | | GGTTCACTATCTCCCGGGACAACTCCAAGAACACGCTGTA |
| | | TCTGCAAATGAATTCACTGCGCGCGGAGGATACCGCTGTG |
| | | TACTACTGCGGTGGCTCCGGTTACGCCCTGCACGATGACT |
| | | ATTACGGCCTTGACGTCTGGGGCCAGGGAACCCTCGTGAC |
| | | TGTGTCCAGC |
| SEQ ID | LCDR1 | TGTSSDVGGYNYVS |
| NO: 95 | (Kabat) | |
| SEQ ID | LCDR2 | DVSNRPS |
| NO: 96 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | DVS |
| NO: 99 | (Chothia) | |
| SEQ ID | LCDR3 | YTSSSTLY |
| NO: | (Chothia) | |
| 100 | | |
| SEQ ID | LCDR1 | SSDVGGYNY |
| NO: | (IMGT) | |
| 101 | | |
| SEQ ID | LCDR2 | DVS |
| NO: 99 | (IMGT) | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHP |
| NO: | | GKAPKLMIYDVSNRPSGVSNRFSGSKSGNTASLTISGLQAED |
| 102 | | EADYYCSSYTSSSTLYVFGSGTKVTVL |
| SEQ ID | DNA VL | CAGAGCGCACTGACTCAGCCGGCATCCGTGTCCGGTAGCC |
| NO: | | CCGGACAGTCGATTACCATCTCCTGTACCGGCACCTCCTC |
| 103 | | CGACGTGGGAGGGTACAACTACGTGTCGTGGTACCAGCA |
| | | GCACCCAGGAAAGGCCCCTAAGTTGATGATCTACGATGTG |
| | | TCAAACCGCCCGTCTGGAGTCTCCAACCGGTTCTCCGGCT |
| | | CCAAGTCCGGCAACACCGCCAGCCTGACCATTAGCGGGCT |
| | | GCAAGCCGAGGATGAGGCCGACTACTACTGCTCGAGCTAC |
| | | ACATCCTCGAGCACCCTCTACGTGTTCGGCTCGGGGACTA |
| | | AGGTCACCGTGCTG |
| SEQ ID | Linker | GGGGSGGGGSGGGGS |
| NO: | | |
| 104 | | |
| SEQ ID | scFv (VH- | QVQLQESGGGVVQPGRSLRLSCAASGFTFSSYGMHWVRQAP |
| NO: | linker-VL) | GKGLEWVAVISYDGSNKYYADSVKGRFTISRDNSKNTLYLQ |
| 105 | | MNSLRAEDTAVYYCGGSGYALHDDYYGLDVWGQGTLVTV |
| | | SSGGGGSGGGGSGGGGSQSALTQPASVSGSPGQSITISCTGTS |
| | | SDVGGYNYVSWYQQHPGKAPKLMIYDVSNRPSGVSNRFSGS |
| | | KSGNTASLTISGLQAEDEADYYCSSYTSSSTLYVFGSGTKVT |
| | | VL |
| SEQ ID | DNA scFv | CAAGTGCAGCTGCAGGAATCCGGTGGCGGAGTCGTGCAG |
| NO: | | CCTGGAAGGAGCCTGAGACTCTCATGCGCCGCGTCAGGGT |
| 106 | | TCACCTTTTCCTCCTACGGGATGCATTGGGTCAGACAGGC |
| | | CCCCGGAAAGGGACTCGAATGGGTGGCTGTGATCAGCTAC |
| | | GACGGCTCCAACAAGTACTACGCCGACTCCGTGAAAGGCC |
| | | GGTTCACTATCTCCCGGGACAACTCCAAGAACACGCTGTA |
| | | TCTGCAAATGAATTCACTGCGCGCGGAGGATACCGCTGTG |
| | | TACTACTGCGGTGGCTCCGGTTACGCCCTGCACGATGACT |
| | | ATTACGGCCTTGACGTCTGGGGCCAGGGAACCCTCGTGAC |
| | | TGTGTCCAGCGGTGGAGGAGGTTCGGGCGGAGGAGGATC |
| | | AGGAGGGGGTGGATCGCAGAGCGCACTGACTCAGCCGGC |
| | | ATCCGTGTCCGGTAGCCCCGGACAGTCGATTACCATCTCC |
| | | TGTACCGGCACCTCCTCCGACGTGGGAGGGTACAACTACG |
| | | TGTCGTGGTACCAGCAGCACCCAGGAAAGGCCCCTAAGTT |
| | | GATGATCTACGATGTGTCAAACCGCCCGTCTGGAGTCTCC |
| | | AACCGGTTCTCCGGCTCCAAGTCCGGCAACACCGCCAGCC |
| | | TGACCATTAGCGGGCTGCAAGCCGAGGATGAGGCCGACT |
| | | ACTACTGCTCGAGCTACACATCCTCGAGCACCCTCTACGT |
| | | GTTCGGCTCGGGGACTAAGGTCACCGTGCTG |
| SEQ ID | Full CAR | QVQLQESGGGVVQPGRSLRLSCAASGFTFSSYGMHWVRQAP |
| NO: | amino acid | GKGLEWVAVISYDGSNKYYADSVKGRFTISRDNSKNTLYLQ |
| 107 | sequence | MNSLRAEDTAVYYCGGSGYALHDDYYGLDVWGQGTLVTV |
| | | SSGGGGSGGGGSGGGGSQSALTQPASVSGSPGQSITISCTGTS |
| | | SDVGGYNYVSWYQQHPGKAPKLMIYDVSNRPSGVSNRFSGS |
| | | KSGNTASLTISGLQAEDEADYYCSSYTSSSTLYVFGSGTKVT |
| | | VLTTTPAPRPPTPAPTIASQPLSLRPEACRPAAGGAVHTRGLD |
| | | FACDIYIWAPLAGTCGVLLLSLVITLYCKRGRKKLLYIFKQPF |
| | | MRPVQTTQEEDGCSCRFPEEEEGGCELRVKFSRSADAPAYQ |
| | | QGQNQLYNELNLGRREEYDVLDKRRGRDPEMGGKPRRKNP |
| | | QEGLYNELQKDKMAEAYSEIGMKGERRRGKGHDGLYQGLS |
| | | TATKDTYDALHMQALPPR |
| SEQ ID | Full CAR | CAAGTGCAGCTGCAGGAATCCGGTGGCGGAGTCGTGCAG |
| NO: | DNA | CCTGGAAGGAGCCTGAGACTCTCATGCGCCGCGTCAGGGT |
| 108 | sequence | TCACCTTTTCCTCCTACGGGATGCATTGGGTCAGACAGGC |
| | | CCCCGGAAAGGGACTCGAATGGGTGGCTGTGATCAGCTAC |
| | | GACGGCTCCAACAAGTACTACGCCGACTCCGTGAAAGGCC |
| | | GGTTCACTATCTCCCGGGACAACTCCAAGAACACGCTGTA |
| | | TCTGCAAATGAATTCACTGCGCGCGGAGGATACCGCTGTG |
| | | TACTACTGCGGTGGCTCCGGTTACGCCCTGCACGATGACT |
| | | ATTACGGCCTTGACGTCTGGGGCCAGGGAACCCTCGTGAC |
| | | TGTGTCCAGCGGTGGAGGAGGTTCGGGCGGAGGAGGATC |
| | | AGGAGGGGGTGGATCGCAGAGCGCACTGACTCAGCCGGC |
| | | ATCCGTGTCCGGTAGCCCCGGACAGTCGATTACCATCTCC |
| | | TGTACCGGCACCTCCTCCGACGTGGGAGGGTACAACTACG |
| | | TGTCGTGGTACCAGCAGCACCCAGGAAAGGCCCCTAAGTT |
| | | GATGATCTACGATGTGTCAAACCGCCCGTCTGGAGTCTCC |
| | | AACCGGTTCTCCGGCTCCAAGTCCGGCAACACCGCCAGCC |
| | | TGACCATTAGCGGGCTGCAAGCCGAGGATGAGGCCGACT |
| | | ACTACTGCTCGAGCTACACATCCTCGAGCACCCTCTACGT |
| | | GTTCGGCTCGGGGACTAAGGTCACCGTGCTGACCACTACC |
| | | CCAGCACCGAGGCCACCCACCCCGGCTCCTACCATCGCCT |
| | | CCCAGCCTCTGTCCCTGCGTCCGGAGGCATGTAGACCCGC |
| | | AGCTGGTGGGGCCGTGCATACCCGGGGTCTTGACTTCGCC |
| | | TGCGATATCTACATTTGGGCCCCTCTGGCTGGTACTTGCGG |
| | | GGTCCTGCTGCTTTCACTCGTGATCACTCTTTACTGTAAGC |
| | | GCGGTCGGAAGAAGCTGCTGTACATCTTTAAGCAACCCTT |
| | | CATGAGGCCTGTGCAGACTACTCAAGAGGAGGACGGCTG |
| | | TTCATGCCGGTTCCCAGAGGAGGAGGAAGGCGGCTGCGA |
| | | ACTGCGCGTGAAATTCAGCCGCAGCGCAGATGCTCCAGCC |
| | | TACCAGCAGGGGCAGAACCAGCTCTACAACGAACTCAAT |
| | | CTTGGTCGGAGAGAGGAGTACGACGTGCTGGACAAGCGG |
| | | AGAGGACGGGACCCAGAAATGGGCGGGAAGCCGCGCAGA |
| | | AAGAATCCCCAAGAGGGCCTGTACAACGAGCTCCAAAAG |
| | | GATAAGATGGCAGAAGCCTATAGCGAGATTGGTATGAAA |
| | | GGGGAACGCAGAAGAGGCAAAGGCCACGACGGACTGTAC |
| | | CAGGGACTCAGCACCGCCACcaaggacacctatgacgct |
| | | atcacatgcaggccctgccgcctcgg |
| B61-02 | | |
| SEQ ID | HCDR1 | SYGMH |
| NO: 86 | (Kabat) | |
| SEQ ID | HCDR2 | VISYKGSNKYYADSVKG |
| NO: | (Kabat) | |
| 109 | | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: | (Chothia) | |
| 110 | | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: | (IMGT) | |
| 111 | | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAP |
| NO: | | GKGLEWVAVISYKGSNKYYADSVKGRFTISRDNSKNTLYLQ |
| 112 | | MNSLRAEDTAVYYCGGSGYALHDDYYGLDVWGQGTLVTV |
| | | SS |
| SEQ ID | DNA VH | CAAGTGCAGCTTGTCGAATCGGGAGGCGGAGTGGTGCAG |
| NO: | | CCTGGACGATCGCTCCGGCTCTCATGTGCCGCGAGCGGAT |
| 113 | | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCT |
| SEQ ID | LCDR1 | TGTSSDVGGYNYVS |
| NO: 95 | (Kabat) | |
| SEQ ID | LCDR2 | EVSNRLR |
| NO: | (Kabat) | |
| 114 | | |
| SEQ ID | LCDR3 | SSYTSSSALYV |
| NO: | (Kabat) | |
| 115 | | |
| SEQ ID | LCDR1 | TSSDVGGYNY |
| NO: 98 | (Chothia) | |
| SEQ ID | LCDR2 | EVS |
| NO: | (Chothia) | |
| 116 | | |
| SEQ ID | LCDR3 | YTSSSALY |
| NO: | (Chothia) | |
| 117 | | |
| SEQ ID | LCDR1 | SSDVGGYNY |
| NO: | (IMGT) | |
| 101 | | |
| SEQ ID | LCDR2 | EVS |
| NO: | (IMGT) | |
| 116 | | |
| SEQ ID | LCDR3 | SSYTSSSALYV |
| NO: | (IMGT) | |
| 115 | | |
| SEQ ID | VL | QSALTQPASVSGSPGQSITISCTGTSSDVGGYNYVSWYQQHP |
| NO: | | GKAPKLMIYEVSNRLRGVSNRFSGSKSGNTASLTISGLQAED |
| 118 | | EADYYCSSYTSSSALYVFGSGTKVTVL |
| SEQ ID | DNA VL | CAGAGCGCGCTGACTCAGCCTGCCTCCGTGAGCGGTTCGC |
| NO: | | CGGGACAGTCCATTACCATTTCGTGCACCGGGACCTCCTC |
| 119 | | CGACGTGGGAGGCTACAACTACGTGTCCTGGTACCAGCAG |
| | | CATCCCGGAAAGGCCCCGAAGCTGATGATCTACGAAGTGT |
| | | CGAACAGACTGCGGGGAGTCTCCAACCGCTTTTCCGGGTC |
| | | CAAGTCCGGCAACACCGCCAGCCTGACCATCAGCGGGCTC |
| | | CAGGCAGAAGATGAGGCTGACTATTACTGCTCCTCCTACA |
| | | CGTCAAGCTCCGCCCTCTACGTGTTCGGGTCCGGGACCAA |
| | | AGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAP |
| NO: | linker-VL) | GKGLEWVAVISYKGSNKYYADSVKGRFTISRDNSKNTLYLQ |
| 120 | | MNSLRAEDTAVYYCGGSGYALHDDYYGLDVWGQGTLVTV |
| | | SSGGGGSGGGGSGGGGSGGGGSQSALTQPASVSGSPGQSITI |
| | | SCTGTSSDVGGYNYVSWYQQHPGKAPKLMIYEVSNRLRGVS |
| | | NRFSGSKSGNTASLTISGLQAEDEADYYCSSYTSSSALYVFGS |
| | | GTKVTVL |
| SEQ ID | DNA scFv | CAAGTGCAGCTTGTCGAATCGGGAGGCGGAGTGGTGCAG |
| NO: | | CCTGGACGATCGCTCCGGCTCTCATGTGCCGCGAGCGGAT |
| 121 | | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCTGGTGGAGGCGGATCAGGGGGTGGCGGAT |
| | | CTGGGGGTGGTGGTTCCGGGGGAGGAGGATCGCAGAGCG |
| | | CGCTGACTCAGCCTGCCTCCGTGAGCGGTTCGCCGGGACA |
| | | GTCCATTACCATTTCGTGCACCGGGACCTCCTCCGACGTG |
| | | GGAGGCTACAACTACGTGTCCTGGTACCAGCAGCATCCCG |
| | | GAAAGGCCCCGAAGCTGATGATCTACGAAGTGTCGAACA |
| | | GACTGCGGGGAGTCTCCAACCGCTTTTCCGGGTCCAAGTC |
| | | CGGCAACACCGCCAGCCTGACCATCAGCGGGCTCCAGGC |
| | | AGAAGATGAGGCTGACTATTACTGCTCCTCCTACACGTCA |
| | | AGCTCCGCCCTCTACGTGTTCGGGTCCGGGACCAAAGTCA |
| | | CTGTGCTG |
| SEQ ID | Full CAR | QVQLVESGGGVVQPGRSLRLSCAASGFTFSSYGMHWVRQAP |
| NO: | amino acid | GKGLEWVAVISYKGSNKYYADSVKGRFTISRDNSKNTLYLQ |
| 122 | sequence | MNSLRAEDTAVYYCGGSGYALHDDYYGLDVWGQGTLVTV |
| | | SSGGGGSGGGGSGGGGSGGGGSQSALTQPASVSGSPGQSITI |
| | | SCTGTSSDVGGYNYVSWYQQHPGKAPKLMIYEVSNRLRGVS |
| | | NRFSGSKSGNTASLTISGLQAEDEADYYCSSYTSSSALYVFGS |
| | | GTKVTVLTTTPAPRPPTPAPTIASQPLSLRPEACRPAAGGAVH |
| | | TRGLDFACDIYIWAPLAGTCGVLLLSLVITLYCKRGRKKLLYI |
| | | FKQPFMRPVQTTQEEDGCSCRFPEEEEGGCELRVKFSRSADA |
| | | PAYQQGQNQLYNELNLGRREEYDVLDKRRGRDPEMGGKPR |
| | | RKNPQEGLYNELQKDKMAEAYSEIGMKGERRRGKGHDGLY |
| | | QGLSTATKDTYDALHMQALPPR |
| SEQ ID | Full CAR | CAAGTGCAGCTTGTCGAATCGGGAGGCGGAGTGGTGCAG |
| NO: | DNA | CCTGGACGATCGCTCCGGCTCTCATGTGCCGCGAGCGGAT |
| 123 | sequence | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCTGGTGGAGGCGGATCAGGGGGTGGCGGAT |
| | | CTGGGGGTGGTGGTTCCGGGGGAGGAGGATCGCAGAGCG |
| | | CGCTGACTCAGCCTGCCTCCGTGAGCGGTTCGCCGGGACA |
| | | GTCCATTACCATTTCGTGCACCGGGACCTCCTCCGACGTG |
| | | GGAGGCTACAACTACGTGTCCTGGTACCAGCAGCATCCCG |
| | | GAAAGGCCCCGAAGCTGATGATCTACGAAGTGTCGAACA |
| | | GACTGCGGGGAGTCTCCAACCGCTTTTCCGGGTCCAAGTC |
| | | CGGCAACACCGCCAGCCTGACCATCAGCGGGCTCCAGGC |
| | | AGAAGATGAGGCTGACTATTACTGCTCCTCCTACACGTCA |
| | | AGCTCCGCCCTCTACGTGTTCGGGTCCGGGACCAAAGTCA |
| | | CTGTGCTGACCACTACCCCAGCACCGAGGCCACCCACCCC |
| | | GGCTCCTACCATCGCCTCCCAGCCTCTGTCCCTGCGTCCGG |
| | | AGGCATGTAGACCCGCAGCTGGTGGGGCCGTGCATACCCG |
| | | GGGTCTTGACTTCGCCTGCGATATCTACATTTGGGCCCCTC |
| | | TGGCTGGTACTTGCGGGGTCCTGCTGCTTTCACTCGTGATC |
| | | ACTCTTTACTGTAAGCGCGGTCGGAAGAAGCTGCTGTACA |
| | | TCTTTAAGCAACCCTTCATGAGGCCTGTGCAGACTACTCA |
| | | AGAGGAGGACGGCTGTTCATGCCGGTTCCCAGAGGAGGA |
| | | GGAAGGCGGCTGCGAACTGCGCGTGAAATTCAGCCGCAG |
| | | CGCAGATGCTCCAGCCTACCAGCAGGGGCAGAACCAGCT |
| | | CTACAACGAACTCAATCTTGGTCGGAGAGAGGAGTACGA |
| | | CGTGCTGGACAAGCGGAGAGGACGGGACCCAGAAATGGG |
| | | CGGGAAGCCGCGCAGAAAGAATCCCCAAGAGGGCCTGTA |
| | | CAACGAGCTCCAAAAGGATAAGATGGCAGAAGCCTATAG |
| | | CGAGATTGGTATGAAAGGGGAACGCAGAAGAGGCAAAGG |
| | | CCACGACGGACTGTACCAGGGACTCAGCACCGCCACCAA |
| | | GGACACCTATGACGCTCTTCACATGCAGGCCCTGCCGCCT |
| | | CGG |
| B61-10 | | |
| SEQ ID | HCDR1 | SYGMH |
| NO: 86 | (Kabat) | |
| SEQ ID | HCDR2 | VISYKGSNKYYADSVKG |
| NO: | (Kabat) | |
| 109 | | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: | (Chothia) | |
| 110 | | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: | (IMGT) | |
| lll | | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAP |
| NO: | | GKGLEWVAVISYKGSNKYYADSVKGRFTISRDNSKNTLYLQ |
| 112 | | MNSLRAEDTAVYYCGGSGYALHDDYYGLDVWGQGTLVTV |
| | | SS |
| SEQ ID | DNA VH | CAAGTGCAGCTTGTCGAATCGGGAGGCGGAGTGGTGCAG |
| NO: | | CCTGGACGATCGCTCCGGCTCTCATGTGCCGCGAGCGGAT |
| 113 | | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCT |
| SEQ ID | LCDR1 | TGTSSDVGGYNYVS |
| NO: 95 | (Kabat) | |
| SEQ ID | LCDR2 | EVSNRLR |
| NO: | (Kabat) | |
| 114 | | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | EVS |
| NO: | (Chothia) | |
| 116 | | |
| SEQ ID | LCDR3 | YTSSSTLY |
| NO: | (Chothia) | |
| 100 | | |
| SEQ ID | LCDR1 | SSDVGGYNY |
| NO: | (IMGT) | |
| 101 | | |
| SEQ ID | LCDR2 | EVS |
| NO: | (IMGT) | |
| 116 | | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHP |
| NO: | | GKAPKLMIYEVSNRLRGVSNRFSGSKSGNTASLTISGLQAED |
| 124 | | EADYYCSSYTSSSTLYVFGSGTKVTVL |
| SEQ ID | DNA VL | CAGAGCGCGCTGACTCAGCCTGCCTCCGTGAGCGGTTCGC |
| NO: | | CGGGACAGTCCATTACCATTTCGTGCACCGGGACCTCCTC |
| 125 | | CGACGTGGGAGGCTACAACTACGTGTCCTGGTACCAGCAG |
| | | CATCCCGGAAAGGCCCCGAAGCTGATGATCTACGAAGTGT |
| | | CGAACAGACTGCGGGGAGTCTCCAACCGCTTTTCCGGGTC |
| | | CAAGTCCGGCAACACCGCCAGCCTGACCATCAGCGGGCTC |
| | | CAGGCAGAAGATGAGGCTGACTATTACTGCTCCTCCTACA |
| | | CGTCAAGCTCCACCCTCTACGTGTTCGGGTCCGGGACCAA |
| | | AGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAP |
| NO: | linker-VL) | GKGLEWVAVISYKGSNKYYADSVKGRFTISRDNSKNTLYLQ |
| 126 | | MNSLRAEDTAVYYCGGSGYALHDDYYGLDVWGQGTLVTV |
| | | SSGGGGSGGGGSGGGGSGGGGSQSALTQPASVSGSPGQSITI |
| | | SCTGTSSDVGGYNYVSWYQQHPGKAPKLMIYEVSNRLRGVS |
| | | NRFSGSKSGNTASLTISGLQAEDEADYYCSSYTSSSTLYVFGS |
| | | GTKVTVL |
| SEQ ID | DNA scFv | CAAGTGCAGCTTGTCGAATCGGGAGGCGGAGTGGTGCAG |
| NO: | | CCTGGACGATCGCTCCGGCTCTCATGTGCCGCGAGCGGAT |
| 127 | | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCTGGTGGAGGCGGATCAGGGGGTGGCGGAT |
| | | CTGGGGGTGGTGGTTCCGGGGGAGGAGGATCGCAGAGCG |
| | | CGCTGACTCAGCCTGCCTCCGTGAGCGGTTCGCCGGGACA |
| | | GTCCATTACCATTTCGTGCACCGGGACCTCCTCCGACGTG |
| | | GGAGGCTACAACTACGTGTCCTGGTACCAGCAGCATCCCG |
| | | GAAAGGCCCCGAAGCTGATGATCTACGAAGTGTCGAACA |
| | | GACTGCGGGGAGTCTCCAACCGCTTTTCCGGGTCCAAGTC |
| | | CGGCAACACCGCCAGCCTGACCATCAGCGGGCTCCAGGC |
| | | AGAAGATGAGGCTGACTATTACTGCTCCTCCTACACGTCA |
| | | AGCTCCACCCTCTACGTGTTCGGGTCCGGGACCAAAGTCA |
| | | CTGTGCTG |
| SEQ ID | Full CAR | QVQLVESGGGVVQPGRSLRLSCAASGFTFSSYGMHWVRQAP |
| NO: | amino acid | GKGLEWVAVISYKGSNKYYADSVKGRFTISRDNSKNTLYLQ |
| 128 | sequence | MNSLRAEDTAVYYCGGSGYALHDDYYGLDVWGQGTLVTV |
| | | SSGGGGSGGGGSGGGGSGGGGSQSALTQPASVSGSPGQSITI |
| | | SCTGTSSDVGGYNYVSWYQQHPGKAPKLMIYEVSNRLRGVS |
| | | NRFSGSKSGNTASLTISGLQAEDEADYYCSSYTSSSTLYVFGS |
| | | GTKVTVLTTTPAPRPPTPAPTIASQPLSLRPEACRPAAGGAVH |
| | | TRGLDFACDIYIWAPLAGTCGVLLLSLVITLYCKRGRKKLLYI |
| | | FKQPFMRPVQTTQEEDGCSCRFPEEEEGGCELRVKFSRSADA |
| | | PAYQQGQNQLYNELNLGRREEYDVLDKRRGRDPEMGGKPR |
| | | RKNPQEGLYNELQKDKMAEAYSEIGMKGERRRGKGHDGLY |
| | | QGLSTATKDTYDALHMQALPPR |
| SEQ ID | Full CAR | CAAGTGCAGCTTGTCGAATCGGGAGGCGGAGTGGTGCAG |
| NO: | DNA | CCTGGACGATCGCTCCGGCTCTCATGTGCCGCGAGCGGAT |
| 129 | sequence | TCACCTTCTCGAGCTACGGCATGCACTGGGTCAGACAAGC |
| | | CCCAGGAAAGGGCCTGGAATGGGTGGCTGTCATCTCGTAC |
| | | AAGGGCTCAAACAAGTACTACGCCGACTCCGTGAAGGGC |
| | | CGGTTCACCATCTCCCGCGATAACTCCAAGAATACCCTCT |
| | | ATCTGCAAATGAACAGCCTGAGGGCCGAGGATACTGCAG |
| | | TGTACTACTGCGGGGGTTCAGGCTACGCGCTGCACGACGA |
| | | CTACTACGGATTGGACGTCTGGGGCCAAGGAACTCTTGTG |
| | | ACCGTGTCCTCTGGTGGAGGCGGATCAGGGGGTGGCGGAT |
| | | CTGGGGGTGGTGGTTCCGGGGGAGGAGGATCGCAGAGCG |
| | | CGCTGACTCAGCCTGCCTCCGTGAGCGGTTCGCCGGGACA |
| | | GTCCATTACCATTTCGTGCACCGGGACCTCCTCCGACGTG |
| | | GGAGGCTACAACTACGTGTCCTGGTACCAGCAGCATCCCG |
| | | GAAAGGCCCCGAAGCTGATGATCTACGAAGTGTCGAACA |
| | | GACTGCGGGGAGTCTCCAACCGCTTTTCCGGGTCCAAGTC |
| | | CGGCAACACCGCCAGCCTGACCATCAGCGGGCTCCAGGC |
| | | AGAAGATGAGGCTGACTATTACTGCTCCTCCTACACGTCA |
| | | AGCTCCACCCTCTACGTGTTCGGGTCCGGGACCAAAGTCA |
| | | CTGTGCTGACCACTACCCCAGCACCGAGGCCACCCACCCC |
| | | GGCTCCTACCATCGCCTCCCAGCCTCTGTCCCTGCGTCCGG |
| | | AGGCATGTAGACCCGCAGCTGGTGGGGCCGTGCATACCCG |
| | | GGGTCTTGACTTCGCCTGCGATATCTACATTTGGGCCCCTC |
| | | TGGCTGGTACTTGCGGGGTCCTGCTGCTTTCACTCGTGATC |
| | | ACTCTTTACTGTAAGCGCGGTCGGAAGAAGCTGCTGTACA |
| | | TCTTTAAGCAACCCTTCATGAGGCCTGTGCAGACTACTCA |
| | | AGAGGAGGACGGCTGTTCATGCCGGTTCCCAGAGGAGGA |
| | | GGAAGGCGGCTGCGAACTGCGCGTGAAATTCAGCCGCAG |
| | | CGCAGATGCTCCAGCCTACCAGCAGGGGCAGAACCAGCT |
| | | CTACAACGAACTCAATCTTGGTCGGAGAGAGGAGTACGA |
| | | CGTGCTGGACAAGCGGAGAGGACGGGACCCAGAAATGGG |
| | | CGGGAAGCCGCGCAGAAAGAATCCCCAAGAGGGCCTGTA |
| | | CAACGAGCTCCAAAAGGATAAGATGGCAGAAGCCTATAG |
| | | CGAGATTGGTATGAAAGGGGAACGCAGAAGAGGCAAAGG |
| | | CCACGACGGACTGTACCAGGGACTCAGCACCGCCACCAA |
| | | GGACACCTATGACGCTCTTCACATGCAGGCCCTGCCGCCT |
| | | CGG |

TABLE 7
Kabat CDRs of exemplary B cell-derived anti-BCMA molecules

| Kabat | HCDR1 | HCDR2 | HCDR3 | LCDR1 | LCDR2 | LCDR3 |
|---|---|---|---|---|---|---|
| PI61 | SYGMH | VISYDGSNK | SGYALHD | TGTSSDV | DVSNRPS | SSYTSSS |
| | (SEQ ID | YYADSVKG | DYYGLDV | GGYNYVS | (SEQ ID | TLYV |
| | NO: 86) | (SEQ ID | (SEQ ID | (SEQ ID | NO: 96) | (SEQ ID |
| | | NO: 87) | NO: 88) | NO: 95) | | NO: 97) |
| B61-02 | SYGMH | VISYKGSNK | SGYALHD | TGTSSDV | EVSNRLR | SSYTSSS |
| | (SEQ ID | YYADSVKG | DYYGLDV | GGYNYVS | (SEQ ID | ALYV |
| | NO: 86) | (SEQ ID | (SEQ ID | (SEQ ID | NO: 114) | (SEQ ID |
| | | NO: 109) | NO: 88) | NO: 95) | | NO: 115) |
| B61-10 | SYGMH | VISYKGSNK | SGYALHD | TGTSSDV | EVSNRLR | SSYTSSS |
| | (SEQ ID | YYADSVKG | DYYGLDV | GGYNYVS | (SEQ ID | TLYV |
| | NO: 86) | (SEQ ID | (SEQ ID | (SEQ ID | NO: 114) | (SEQ ID |
| | | NO: 109) | NO: 88) | NO: 95) | | NO: 97) |
| Consen- | SYGMH | VISYXGSNK | SGYALHD | TGTSSDV | X₁VSNRX₂X₃, | SSYTSSS |
| sus | (SEQ ID | YYADSVKG, | DYYGLDV | GGYNYVS | wherein X₁ | XLYV, |
| | NO: 86) | wherein X | (SEQ ID | (SEQ ID | is D or E; | wherein |
| | | is D or K | NO: 88) | NO: 95) | X₂ is P or | X is T |
| | | (SEQ ID | | | L; and X₃ | or A |
| | | NO: 130) | | | is S or R | (SEQ ID |
| | | | | | (SEQ ID | NO: 132) |
| | | | | | NO: 131) | |

TABLE 8
Chothia CDRs of exemplary B cell-derived anti-BCMA molecules

| Chothia | HCDR1 | HCDR2 | HCDR3 | LCDR1 | LCDR2 | LCDR3 |
|---|---|---|---|---|---|---|
| PI61 | GFTFSSY | SYDGSN | SGYALHDDY | TSSDVGG | DVS | YTSSSTLY |
| | (SEQ ID | (SEQ ID | YGLDV | YNY | (SEQ ID | (SEQ ID |
| | NO: 47) | NO: 89) | (SEQ ID | (SEQ ID | NO: 99) | NO: 100) |
| | | | NO: 88) | NO: 98) | | |
| B61-02 | GFTFSSY | SYKGSN | SGYALHDDY | TSSDVGG | EVS | YTSSSALY |
| | (SEQ ID | (SEQ ID | YGLDV | YNY | (SEQ ID | (SEQ ID |
| | NO: 47) | NO: 110) | (SEQ ID | (SEQ ID | NO: 116) | NO: 117) |
| | | | NO: 88) | NO: 98) | | |
| B61-10 | GFTFSSY | SYKGSN | SGYALHDDY | TSSDVGG | EVS | YTSSSTLY |
| | (SEQ ID | (SEQ ID | YGLDV | YNY | (SEQ ID | SEQ ID |
| | NO: 47) | NO: 110) | (SEQ ID | (SEQ ID | NO: 116) | NO: 100) |
| | | | NO: 88) | NO: 98) | | |
| Consen- | GFTFSSY | SYXGSN, | SGYALHDDY | TSSDVGG | XVS, | YTSSSXLY, |
| sus | (SEQ ID | wherein | YGLDV | YNY | wherein | wherein |
| | NO: 47) | X is D | (SEQ ID | (SEQ ID | X is D | X is T |
| | | or K | NO: 88) | NO: 98) | or E | or A (SEQ |
| | | (SEQ ID | | | (SEQ ID | ID NO: |
| | | NO: 133) | | | NO: 134) | 135) |

TABLE 9
IMGT CDRs of exemplary B cell-derived anti-BCMA molecules

| IMGT | HCDR1 | HCDR2 | HCDR3 | LCDR1 | LCDR2 | LCDR3 |
|---|---|---|---|---|---|---|
| PI61 | GFTFSSY | ISYDGSNK | GGSGYALHD | SSDVGGY | DVS | SSYTSSSTL |
| | G (SEQ | (SEQ ID | DYYGLDV | NY (SEQ | (SEQ ID | YV (SEQ ID |
| | ID NO: | NO: 91) | (SEQ ID | ID NO: | NO: 99) | NO: 97) |
| | 90) | | NO: 92) | 101) | | |
| B61- | GFTFSSY | ISYKGSNK | GGSGYALHD | SSDVGGY | EVS | SSYTSSSAL |
| 02 | G (SEQ | (SEQ ID | DYYGLDV | NY (SEQ | (SEQ ID | YV (SEQ ID |
| | ID NO: | NO: 111) | (SEQ ID | ID NO: | NO: 116) | NO: 15) |
| | 90) | | NO: 92) | 101) | | |
| B61- | GFTFSSY | ISYKGSNK | GGSGYALHD | SSDVGGY | EVS | SSYTSSSTL |
| 10 | G (SEQ | (SEQ ID | DYYGLDV | NY (SEQ | (SEQ Id | YV (SEQ ID |
| | ID NO: | NO: 111) | (SEQ ID | ID NO: | NO: 116) | NO: 97) |
| | 90) | | NO: 92) | 101) | | |
| Con- | GFTFSSY | ISYXGSNK, | GGSGYALHD | SSDVGGY | XVS, | SSYTSSSXL |
| sen- | G (SEQ | wherein | DYYGLDV | NY (SEQ | wherein | YV, |
| sus | ID NO: | X is D | (SEQ ID | ID NO: | X is D | wherein X |
| | 90) | or K | NO: 92) | 101) | or E | is T or A |
| | | (SEQ ID | | | (SEQ ID | (SEQ ID |
| | | NO: 136) | | | NO: 134) | NO: 132) |

TABLE 14
Amino acid and nucleic acid sequences of exemplary anti-BCMA molecules based
on PI61

| Identification | Protein sequence | DNA sequence (5′-3′) |
|---|---|---|
| Signal peptide | MALPVTALLLPLALLLHA | Atggccctccctgtcaccgctctgttgctgccgcttgctc |
| | ARP (SEQ ID NO: 2) | tgctgctccacgcagcgcgaccg (SEQ ID NO: 252) |
| ScFv PI61 | QVQLQESGGGVVQPGRS | CaggtacaattgcaggagtctggaggcggtgtgGtgca |
| | LRLSCAASGFTFSSYGMH | acccggtcgcagcttgcgcctgagttgtGctgcgtctgg |
| | WVRQAPGKGLEWVAVIS | atttacattttcatcttacggaAtgcattgggtacgccag |
| | YDGSNKYYADSVKGRFT | gcaccggggaaaggcCttgaatgggtggctgtaatttcat |
| | ISRDNSKNTLYLQMNSLR | acgatggtTccaacaaatactatgctgactcagtcaagg |
| | AEDTAVYYCGGSGYALH | gtCgatttacaattagtcgggacaactccaagaacAccc |
| | DDYYGLDVWGQGTLVT | tttatcttcaaatgaattcccttagagcaGaggatacggc |
| | VSSGGGGSGGGGSGGGG | ggtctattactgtggtggcagtGgttatgcacttcatgat |
| | SQSALTQPASVSGSPGQSI | gattactatggcttgGatgtctgggggcaagggacgcttg |
| | TISCTGTSSDVGGYNYVS | taactgtaTcctctggtggtggtggtagtggtgggggagg |
| | WYQQHPGKAPKLMIYDV | cTccggcggtggcggctctcaatctgctctgactCaacca |
| | SNRPSGVSNRFSGSKSGN | gcaagcgtatcagggtcaccgggacagAgtattaccat |
| | TASLTISGLQAEDEADYY | aagttgcacggggacctctagcGatgtaggggggtata |
| | CSSYTSSSTLYVFGSGTK | attatgtatcttggtatCaacaacaccccgggaaagcccc |
| | VTVL(SEQ ID NO: 105) | taaattgatgAtctacgacgtgagcaatcgacctagtggc |
| | | gtaTcaaatcgcttctctggtagcaagagtgggaatAcg |
| | | gcgtcccttactattagcggattgcaagcaGaagatgag |
| | | gccgattactactgcagctcctatActagctcttctacat |
| | | tgtacgtctttgggagcggaacaaaagtaacagtactc |
| | | (SEQ ID NO: 253) |
| Transmembrane | TTTPAPRPPTPAPTIASQP | AcaacaacacctgccccgagaccgcctacaccaGccc |
| domain and | LSLRPEACRPAAGGAVHT | cgactattgccagccagcctctgagcctcAggcctgag |
| hinge | RGLDFACDIYIWAPLAGT | gcctgtaggcccgcagcgggcggcGcagttcatacac |
| | CGVLLLSLVITLYC(SEQ | ggggcttggatttcgcttgtGatatttatatttgggctcc |
| | ID NO: 202) | tttggcggggacaTgtggcgtgctgcttctgtcacttgtt |
| | | attacactgtactgt (SEQ ID NO: 254) |
| 4-1BB | KRGRKKLLYIFKQPFMRP | AaacgcgggcgaaaaaaattgctgtatatttttAagcag |
| | VQTTQEEDGCSCRFPEEE | ccatttatgaggcccgttcagacgacgCaggaggagga |
| | EGGCEL (SEQ ID | cggttgctcttgcaggttcccagaagaggaagaagggg |
| | NO: 14) | gctgtgaattg (SEQ ID NO: 255) |
| CD3zeta | RVKFSRSADAPAYQQGQ | CgggttaaattttcaagatccgcagacgctccaGcatac |
| | NQLYNELNLGRREEYDV | caacagggacaaaaccaactctataacGagctgaatctt |
| | LDKRRGRDPEMGGKPRR | ggaagaagggaggaatatgatGtgctggataaacggcg |
| | KNPQEGLYNELQKDKMA | cggtagagatccggagAtgggcggaaaaccaaggcg |
| | EAYSEIGMKGERRRGKG | aaaaaaccctcagGagggactctacaacgaactgcaga |
| | HDGLYQGLSTATKDTYD | aagacaaaAtggcggaggcttattccgaaataggcatg |
| | ALHMQALPPR(SEQ ID | aagGgcgagcggaggcgagggaaagggcacgacgg |
| | NO: 20) | aCtgtatcaaggcctctcaaccgcgactaaggatAcgta |
| | | cgacgccctgcacatgcaggccctgcctccgaga |
| | | (SEQ ID NO: 256) |
| PI61 full CAR | MALPVTALLLPLALLLHA | ATGGCCCTCCCTGTCACCGCTCTGT |
| construct | ARPQVQLQESGGGVVQP | TGCTGCCGCTTGCTCTGCTGCTCCA |
| | GRSLRLSCAASGFTFSSY | CGCAGCGCGACCGCAGGTACAATT |
| | GMHWVRQAPGKGLEWV | GCAGGAGTCTGGAGGCGGTGTGGT |
| | AVISYDGSNKYYADSVK | GCAACCCGGTCGCAGCTTGCGCCT |
| | GRFTISRDNSKNTLYLQM | GAGTTGTGCTGCGTCTGGATTTACA |
| | NSLRAEDTAVYYCGGSG | TTTTCATCTTACGGAATGCATTGGG |
| | YALHDDYYGLDVWGQG | TACGCCAGGCACCGGGGAAAGGCC |
| | TLVTVSSGGGGSGGGGS | TTGAATGGGTGGCTGTAATTTCATA |
| | GGGGSQSALTQPASVSGS | CGATGGTTCCAACAAATACTATGCT |
| | PGQSITISCTGTSSDVGGY | GACTCAGTCAAGGGTCGATTTACA |
| | NYVSWYQQHPGKAPKL | ATTAGTCGGGACAACTCCAAGAAC |
| | MIYDVSNRPSGVSNRFSG | ACCCTTTATCTTCAAATGAATTCCC |
| | SKSGNTASLTISGLQAED | TTAGAGCAGAGGATACGGCGGTCT |
| | EADYYCSSYTSSSTLYVF | ATTACTGTGGTGGCAGTGGTTATGC |
| | GSGTKVTVLTTTPAPRPP | ACTTCATGATGATTACTATGGCTTG |
| | TPAPTIASQPLSLRPEACR | GATGTCTGGGGGCAAGGGACGCTT |
| | PAAGGAVHTRGLDFACD | GTAACTGTATCCTCTGGTGGTGGTG |
| | IYIWAPLAGTCGVLLLSL | GTAGTGGTGGGGGAGGCTCCGGCG |
| | VITLYCKRGRKKLLYIFK | GTGGCGGCTCTCAATCTGCTCTGAC |
| | QPFMRPVQTTQEEDGCSC | TCAACCAGCAAGCGTATCAGGGTC |
| | RFPEEEEGGCELRVKFSR | ACCGGGACAGAGTATTACCATAAG |
| | SADAPAYQQGQNQLYNE | TTGCACGGGGACCTCTAGCGATGT |
| | LNLGRREEYDVLDKRRG | AGGGGGGTATAATTATGTATCTTGG |
| | RDPEMGGKPRRKNPQEG | TATCAACAACACCCCGGGAAAGCC |
| | LYNELQKDKMAEAYSEI | CCTAAATTGATGATCTACGACGTGA |
| | GMKGERRRGKGHDGLY | GCAATCGACCTAGTGGCGTATCAA |
| | QGLSTATKDTYDALHMQ | ATCGCTTCTCTGGTAGCAAGAGTGG |
| | ALPPR(SEQ ID NO: 257) | GAATACGGCGTCCCTTACTATTAGC |
| | | GGATTGCAAGCAGAAGATGAGGCC |
| | | GATTACTACTGCAGCTCCTATACTA |
| | | GCTCTTCTACATTGTACGTCTTTGG |
| | | GAGCGGAACAAAAGTAACAGTACT |
| | | CACAACAACACCTGCCCCGAGACC |
| | | GCCTACACCAGCCCCGACTATTGCC |
| | | AGCCAGCCTCTGAGCCTCAGGCCT |
| | | GAGGCCTGTAGGCCCGCAGCGGGC |
| | | GGCGCAGTTCATACACGGGGCTTG |
| | | GATTTCGCTTGTGATATTTATATTT |
| | | GGGCTCCTTTGGCGGGGACATGTG |
| | | GCGTGCTGCTTCTGTCACTTGTTAT |
| | | TACACTGTACTGTAAACGCGGGCG |
| | | AAAAAAATTGCTGTATATTTTTAAG |
| | | CAGCCATTTATGAGGCCCGTTCAGA |
| | | CGACGCAGGAGGAGGACGGTTGCT |
| | | CTTGCAGGTTCCCAGAAGAGGAAG |
| | | AAGGGGGCTGTGAATTGCGGGTTA |
| | | AATTTTCAAGATCCGCAGACGCTCC |
| | | AGCATACCAACAGGGACAAAACCA |
| | | ACTCTATAACGAGCTGAATCTTGGA |
| | | AGAAGGGAGGAATATGATGTGCTG |
| | | GATAAACGGCGCGGTAGAGATCCG |
| | | GAGATGGGCGGAAAACCAAGGCGA |
| | | AAAAACCCTCAGGAGGGACTCTAC |
| | | AACGAACTGCAGAAAGACAAAATG |
| | | GCGGAGGCTTATTCCGAAATAGGC |
| | | ATGAAGGGCGAGCGGAGGCGAGG |
| | | GAAAGGGCACGACGGACTGTATCA |
| | | AGGCCTCTCAACCGCGACTAAGGA |
| | | TACGTACGACGCCCTGCACATGCA |
| | | GGCCCTGCCTCCGAGA (SEQ ID |
| | | NO: 258) |
| PI61 mature | QVQLQESGGGVVQPGRS | |
| CAR protein | LRLSCAASGFTFSSYGMH | |
| | WVRQAPGKGLEWVAVIS | |
| | YDGSNKYYADSVKGRFT | |
| | ISRDNSKNTLYLQMNSLR | |
| | AEDTAVYYCGGSGYALH | |
| | DDYYGLDVWGQGTLVT | |
| | VSSGGGGSGGGGSGGGG | |
| | SQSALTQPASVSGSPGQSI | |
| | TISCTGTSSDVGGYNYVS | |
| | WYQQHPGKAPKLMIYDV | |
| | SNRPSGVSNRFSGSKSGN | |
| | TASLTISGLQAEDEADYY | |
| | CSSYTSSSTLYVFGSGTK | |
| | VTVLTTTPAPRPPTPAPTI | |
| | ASQPLSLRPEACRPAAGG | |
| | AVHTRGLDFACDIYIWAP | |
| | LAGTCGVLLLSLVITLYC | |
| | KRGRKKLLYIFKQPFMRP | |
| | VQTTQEEDGCSCRFPEEE | |
| | EGGCELRVKFSRSADAPA | |
| | YQQGQNQLYNELNLGRR | |
| | EEYDVLDKRRGRDPEMG | |
| | GKPRRKNPQEGLYNELQ | |
| | KDKMAEAYSEIGMKGER | |
| | RRGKGHDGLYQGLSTAT | |
| | KDTYDALHMQALPPR | |
| | (SEQ ID NO: 107) | |

TABLE 10
Amino acid and nucleic acid sequences of exemplary hybridoma-derived
anti-BCMA molecules

| | | |
|---|---|---|
| SEQ | Name/ | |
| ID NO | Description | Sequence |

Hy03

| | | |
|---|---|---|
| SEQ ID | HCDR1 | GFWMS |
| NO: 137 | (Kabat) | |
| SEQ ID | HCDR2 | NIKQDGSEKYYVDSVRG |
| NO: 138 | (Kabat) | |
| SEQ ID | HCDR3 | ALDYYGMDV |
| NO: 139 | (Kabat) | |
| SEQ ID | HCDR1 | GFTFSGF |
| NO: 140 | (Chothia) | |
| SEQ ID | HCDR2 | KQDGSE |
| NO: 141 | (Chothia) | |
| SEQ ID | HCDR3 | ALDYYGMDV |
| NO: 139 | (Chothia) | |
| SEQ ID | HCDR1 | GFTFSGFW |
| NO: 142 | (IMGT) | |
| SEQ ID | HCDR2 | IKQDGSEK |
| NO: 143 | (IMGT) | |
| SEQ ID | HCDR3 | ARALDYYGMDV |
| NO: 144 | (IMGT) | |
| SEQ ID | VH | EVQLVESGGGLVQPGGSLRLSCAASGFTFSGFWMSWVRQA |
| NO: 145 | | PGKGLEWVANIKQDGSEKYYVDSVRGRFTISRDNAKNSLYL |
| | | QMNSLRAEDTAVYYCARALDYYGMDVWGQGTTVTVSS |
| SEQ ID | DNA VH | GAAGTGCAACTGGTGGAGAGCGGTGGAGGGCTTGTCCAG |
| NO: 146 | | CCCGGAGGATCGCTGCGGCTGTCCTGTGCTGCGTCCGGGT |
| | | TCACCTTCTCCGGCTTCTGGATGTCCTGGGTCAGACAGGC |
| | | ACCGGGAAAGGGCCTCGAATGGGTGGCCAACATCAAGCA |
| | | GGATGGCTCCGAGAAGTACTACGTCGACTCCGTGAGAGG |
| | | CCGCTTCACCATCTCCCGGGACAACGCCAAGAACTCGCT |
| | | GTACCTCCAAATGAATAGCCTCAGGGCGGAAGATACTGC |
| | | TGTGTATTACTGCGCACGCGCCCTTGACTACTACGGCATG |
| | | GACGTCTGGGGCCAAGGGACCACTGTGACCGTGTCTAGC |
| SEQ ID | LCDR1 | RSSQSLLDSDDGNTYLD |
| NO: 147 | (Kabat) | |
| SEQ ID | LCDR2 | TLSYRAS |
| NO: 148 | (Kabat) | |
| SEQ ID | LCDR3 | TQRLEFPSIT |
| NO: 149 | (Kabat) | |
| SEQ ID | LCDR1 | SQSLLDSDDGNTY |
| NO: 150 | (Chothia) | |
| SEQ ID | LCDR2 | TLS |
| NO: 151 | (Chothia) | |
| SEQ ID | LCDR3 | RLEFPSI |
| NO: 152 | (Chothia) | |
| SEQ ID | LCDR1 | QSLLDSDDGNTY |
| NO: 153 | (IMGT) | |
| SEQ ID | LCDR2 | TLS |
| NO: 151 | (IMGT) | |
| SEQ ID | LCDR3 | TQRLEFPSIT |
| NO: 149 | (IMGT) | |
| SEQ ID | VL | DIVMTQTPLSLPVTPGEPASISCRSSQSLLDSDDGNTYLDWY |
| NO: 154 | | LQKPGQSPRLLIYTLSYRASGVPDRFSGSGSGTDFTLKISRVE |
| | | AEDVGLYYCTQRLEFPSITFGQGTRLEIK |
| SEQ ID | DNA VL | GATATCGTGATGACCCAGACTCCCCTGTCCCTGCCTGTGA |
| NO: 155 | | CTCCCGGAGAACCAGCCTCCATTTCCTGCCGGTCCTCCCA |
| | | GTCCCTGCTGGACAGCGACGACGGCAACACTTACCTGGA |
| | | CTGGTACTTGCAGAAGCCGGGCCAATCGCCTCGCCTGCTG |
| | | ATCTATACCCTGTCATACCGGGCCTCAGGAGTGCCTGACC |
| | | GCTTCTCGGGATCAGGGAGCGGGACCGATTTCACCCTGA |
| | | AAATTTCCCGAGTGGAAGCCGAGGACGTCGGACTGTACT |
| | | ACTGCACCCAGCGCCTCGAATTCCCGTCGATTACGTTTGG |
| | | ACAGGGTACCCGGCTTGAGATCAAG |
| SEQ ID | Linker | GGGGSGGGGSGGGGSGGGGS |
| NO: 63 | | |
| SEQ ID | scFv (VH- | EVQLVESGGGLVQPGGSLRLSCAASGFTFSGFWMSWVRQA |
| NO: 156 | linker-VL) | PGKGLEWVANIKQDGSEKYYVDSVRGRFTISRDNAKNSLYL |
| | | QMNSLRAEDTAVYYCARALDYYGMDVWGQGTTVTVSSG |
| | | GGGSGGGGSGGGGSGGGGSDIVMTQTPLSLPVTPGEPASISC |
| | | RSSQSLLDSDDGNTYLDWYLQKPGQSPRLLIYTLSYRASGV |
| | | PDRFSGSGSGTDFTLKISRVEAEDVGLYYCTQRLEFPSITFGQ |
| | | GTRLEIK |
| SEQ ID | DNA scFv | GAAGTGCAACTGGTGGAGAGCGGTGGAGGGCTTGTCCAG |
| NO: 157 | | CCCGGAGGATCGCTGCGGCTGTCCTGTGCTGCGTCCGGGT |
| | | TCACCTTCTCCGGCTTCTGGATGTCCTGGGTCAGACAGGC |
| | | ACCGGGAAAGGGCCTCGAATGGGTGGCCAACATCAAGCA |
| | | GGATGGCTCCGAGAAGTACTACGTCGACTCCGTGAGAGG |
| | | CCGCTTCACCATCTCCCGGGACAACGCCAAGAACTCGCT |
| | | GTACCTCCAAATGAATAGCCTCAGGGCGGAAGATACTGC |
| | | TGTGTATTACTGCGCACGCGCCCTTGACTACTACGGCATG |
| | | GACGTCTGGGGCCAAGGGACCACTGTGACCGTGTCTAGC |
| | | GGAGGCGGAGGTTCAGGGGGCGGTGGATCAGGCGGAGG |
| | | AGGATCGGGGGGTGGTGGATCGGATATCGTGATGACCCA |
| | | GACTCCCCTGTCCCTGCCTGTGACTCCCGGAGAACCAGCC |
| | | TCCATTTCCTGCCGGTCCTCCCAGTCCCTGCTGGACAGCG |
| | | ACGACGGCAACACTTACCTGGACTGGTACTTGCAGAAGC |
| | | CGGGCCAATCGCCTCGCCTGCTGATCTATACCCTGTCATA |
| | | CCGGGCCTCAGGAGTGCCTGACCGCTTCTCGGGATCAGG |
| | | GAGCGGGACCGATTTCACCCTGAAAATTTCCCGAGTGGA |
| | | AGCCGAGGACGTCGGACTGTACTACTGCACCCAGCGCCT |
| | | CGAATTCCCGTCGATTACGTTTGGACAGGGTACCCGGCTT |
| | | GAGATCAAG |
| SEQ ID | Full CAR | EVQLVESGGGLVQPGGSLRLSCAASGFTFSGFWMSWVRQA |
| NO: 158 | amino acid | PGKGLEWVANIKQDGSEKYYVDSVRGRFTISRDNAKNSLYL |
| | sequence | QMNSLRAEDTAVYYCARALDYYGMDVWGQGTTVTVSSG |
| | | GGGSGGGGSGGGGSGGGGSDIVMTQTPLSLPVTPGEPASISC |
| | | RSSQSLLDSDDGNTYLDWYLQKPGQSPRLLIYTLSYRASGV |
| | | PDRFSGSGSGTDFTLKISRVEAEDVGLYYCTQRLEFPSITFGQ |
| | | GTRLEIKTTTPAPRPPTPAPTIASQPLSLRPEACRPAAGGAVH |
| | | TRGLDFACDIYIWAPLAGTCGVLLLSLVITLYCKRGRKKLLY |
| | | IFKQPFMRPVQTTQEEDGCSCRFPEEEEGGCELRVKFSRSAD |
| | | APAYQQGQNQLYNELNLGRREEYDVLDKRRGRDPEMGGK |
| | | PRRKNPQEGLYNELQKDKMAEAYSEIGMKGERRRGKGHD |
| | | GLYQGLSTATKDTYDALHMQALPPR |
| SEQ ID | Full CAR | GAAGTGCAACTGGTGGAGAGCGGTGGAGGGCTTGTCCAG |
| NO: 159 | DNA | CCCGGAGGATCGCTGCGGCTGTCCTGTGCTGCGTCCGGGT |
| | sequence | TCACCTTCTCCGGCTTCTGGATGTCCTGGGTCAGACAGGC |
| | | ACCGGGAAAGGGCCTCGAATGGGTGGCCAACATCAAGCA |
| | | GGATGGCTCCGAGAAGTACTACGTCGACTCCGTGAGAGG |
| | | CCGCTTCACCATCTCCCGGGACAACGCCAAGAACTCGCT |
| | | GTACCTCCAAATGAATAGCCTCAGGGCGGAAGATACTGC |
| | | TGTGTATTACTGCGCACGCGCCCTTGACTACTACGGCATG |
| | | GACGTCTGGGGCCAAGGGACCACTGTGACCGTGTCTAGC |
| | | GGAGGCGGAGGTTCAGGGGGCGGTGGATCAGGCGGAGG |
| | | AGGATCGGGGGGTGGTGGATCGGATATCGTGATGACCCA |
| | | GACTCCCCTGTCCCTGCCTGTGACTCCCGGAGAACCAGCC |
| | | TCCATTTCCTGCCGGTCCTCCCAGTCCCTGCTGGACAGCG |
| | | ACGACGGCAACACTTACCTGGACTGGTACTTGCAGAAGC |
| | | CGGGCCAATCGCCTCGCCTGCTGATCTATACCCTGTCATA |
| | | CCGGGCCTCAGGAGTGCCTGACCGCTTCTCGGGATCAGG |
| | | GAGCGGGACCGATTTCACCCTGAAAATTTCCCGAGTGGA |
| | | AGCCGAGGACGTCGGACTGTACTACTGCACCCAGCGCCT |
| | | CGAATTCCCGTCGATTACGTTTGGACAGGGTACCCGGCTT |
| | | GAGATCAAGACCACTACCCCAGCACCGAGGCCACCCACC |
| | | CCGGCTCCTACCATCGCCTCCCAGCCTCTGTCCCTGCGTC |
| | | CGGAGGCATGTAGACCCGCAGCTGGTGGGGCCGTGCATA |
| | | CCCGGGGTCTTGACTTCGCCTGCGATATCTACATTTGGGC |
| | | CCCTCTGGCTGGTACTTGCGGGGTCCTGCTGCTTTCACTC |
| | | GTGATCACTCTTTACTGTAAGCGCGGTCGGAAGAAGCTG |
| | | CTGTACATCTTTAAGCAACCCTTCATGAGGCCTGTGCAGA |
| | | CTACTCAAGAGGAGGACGGCTGTTCATGCCGGTTCCCAG |
| | | AGGAGGAGGAAGGCGGCTGCGAACTGCGCGTGAAATTCA |
| | | GCCGCAGCGCAGATGCTCCAGCCTACCAGCAGGGGCAGA |
| | | ACCAGCTCTACAACGAACTCAATCTTGGTCGGAGAGAGG |
| | | AGTACGACGTGCTGGACAAGCGGAGAGGACGGGACCCA |
| | | GAAATGGGCGGGAAGCCGCGCAGAAAGAATCCCCAAGA |
| | | GGGCCTGTACAACGAGCTCCAAAAGGATAAGATGGCAGA |
| | | AGCCTATAGCGAGATTGGTATGAAAGGGGAACGCAGAAG |
| | | AGGCAAAGGCCACGACGGACTGTACCAGGGACTCAGCAC |
| | | CGCCACCAAGGACACCTATGACGCTCTTCACATGCAGGC |
| | | CCTGCCGCCTCGG |

Hy52

| | | |
|---|---|---|
| SEQ ID | HCDR1 | SFRMN |
| NO: 160 | (Kabat) | |
| SEQ ID | HCDR2 | SISSSSSYIYYADSVKG |
| NO: 161 | (Kabat) | |
| SEQ ID | HCDR3 | WLSYYGMDV |
| NO: 162 | (Kabat) | |
| SEQ ID | HCDR1 | GFTFSSF |
| NO: 163 | (Chothia) | |
| SEQ ID | HCDR2 | SSSSSY |
| NO: 164 | (Chothia) | |
| SEQ ID | HCDR3 | WLSYYGMDV |
| NO: 162 | (Chothia) | |
| SEQ ID | HCDR1 | GFTFSSFR |
| NO: 165 | (IMGT) | |
| SEQ ID | HCDR2 | ISSSSSYI |
| NO: 166 | (IMGT) | |
| SEQ ID | HCDR3 | ARWLSYYGMDV |
| NO: 167 | (IMGT) | |
| SEQ ID | VH | EVQLVESGGGLVKPGGSLRLSCAASGFTFSSFRMNWVRQAP |
| NO: 168 | | GKGLEWVSSISSSSSYIYYADSVKGRFTISRDNAKNSLYLQM |
| | | NSLRAEDTAVYYCARWLSYYGMDVWGQGTTVTVSS |
| SEQ ID | DNA VH | GAAGTGCAACTGGTGGAGAGCGGTGGAGGGCTTGTCAAG |
| NO: 169 | | CCCGGAGGATCGCTGCGGCTGTCCTGTGCTGCGTCCGGGT |
| | | TCACCTTCTCCTCGTTCCGCATGAACTGGGTCAGACAGGC |
| | | ACCGGGAAAGGGCCTCGAATGGGTGTCCTCAATCTCATC |
| | | GTCCTCGTCCTACATCTACTACGCCGACTCCGTGAAAGGC |
| | | CGCTTCACCATCTCCCGGGACAACGCCAAGAACTCGCTGT |
| | | ACCTCCAAATGAATAGCCTCAGGGCGGAAGATACTGCTG |
| | | TGTATTACTGCGCACGCTGGCTTTCCTACTACGGCATGGA |
| | | CGTCTGGGGCCAAGGGACCACTGTGACCGTGTCTAGC |
| SEQ ID | LCDR1 | RSSQSLLDSDDGNTYLD |
| NO: 147 | (Kabat) | |
| SEQ ID | LCDR2 | TLSFRAS |
| NO: 170 | (Kabat) | |
| SEQ ID | LCDR3 | MQRIGFPIT |
| NO: 171 | (Kabat) | |
| SEQ ID | LCDR1 | SQSLLDSDDGNTY |
| NO: 150 | (Chothia) | |
| SEQ ID | LCDR2 | TLS |
| NO: 151 | (Chothia) | |
| SEQ ID | LCDR3 | RIGFPI |
| NO: 172 | (Chothia) | |
| SEQ ID | LCDR1 | QSLLDSDDGNTY |
| NO: 153 | (IMGT) | |
| SEQ ID | LCDR2 | TLS |
| NO: 151 | (IMGT) | |
| SEQ ID | LCDR3 | MQRIGFPIT |
| NO: 171 | (IMGT) | |
| SEQ ID | VL | DIVMTQTPLSLPVTPGEPASISCRSSQSLLDSDDGNTYLDWY |
| NO: 173 | | LQKPGQSPQLLIYTLSFRASGVPDRFSGSGSGTDFTLKIRRVE |
| | | AEDVGVYYCMQRIGFPITFGQGTRLEIK |
| SEQ ID | DNA VL | GATATCGTGATGACCCAGACTCCCCTGTCCCTGCCTGTGA |
| NO: 174 | | CTCCCGGAGAACCAGCCTCCATTTCCTGCCGGTCCTCCCA |
| | | GTCCCTGCTGGACAGCGACGACGGCAACACTTACCTGGA |
| | | CTGGTACTTGCAGAAGCCGGGCCAATCGCCTCAGCTGCT |
| | | GATCTATACCCTGTCATTCCGGGCCTCAGGAGTGCCTGAC |
| | | CGCTTCTCGGGATCAGGGAGCGGGACCGATTTCACCCTG |
| | | AAAATTAGGCGAGTGGAAGCCGAGGACGTCGGAGTGTAC |
| | | TACTGCATGCAGCGCATCGGCTTCCCGATTACGTTTGGAC |
| | | AGGGTACCCGGCTTGAGATCAAG |
| SEQ ID | Linker | GGGGSGGGGSGGGGSGGGGS |
| NO: 63 | | |
| SEQ ID | scFv (VH- | EVQLVESGGGLVKPGGSLRLSCAASGFTFSSFRMNWVRQAP |
| NO: 175 | linker-VL) | GKGLEWVSSISSSSSYIYYADSVKGRFTISRDNAKNSLYLQM |
| | | NSLRAEDTAVYYCARWLSYYGMDVWGQGTTVTVSSGGGG |
| | | SGGGGSGGGGSGGGGSDIVMTQTPLSLPVTPGEPASISCRSS |
| | | QSLLDSDDGNTYLDWYLQKPGQSPQLLIYTLSFRASGVPDR |
| | | FSGSGSGTDFTLKIRRVEAEDVGVYYCMQRIGFPITFGQGTR |
| | | LEIK |
| SEQ ID | DNA scFv | GAAGTGCAACTGGTGGAGAGCGGTGGAGGGCTTGTCAAG |
| NO: 176 | | CCCGGAGGATCGCTGCGGCTGTCCTGTGCTGCGTCCGGGT |
| | | TCACCTTCTCCTCGTTCCGCATGAACTGGGTCAGACAGGC |
| | | ACCGGGAAAGGGCCTCGAATGGGTGTCCTCAATCTCATC |
| | | GTCCTCGTCCTACATCTACTACGCCGACTCCGTGAAAGGC |
| | | CGCTTCACCATCTCCCGGGACAACGCCAAGAACTCGCTGT |
| | | ACCTCCAAATGAATAGCCTCAGGGCGGAAGATACTGCTG |
| | | TGTATTACTGCGCACGCTGGCTTTCCTACTACGGCATGGA |
| | | CGTCTGGGGCCAAGGGACCACTGTGACCGTGTCTAGCGG |
| | | AGGCGGAGGTTCAGGGGGCGGTGGATCAGGCGGAGGAG |
| | | GATCGGGGGGTGGTGGATCGGATATCGTGATGACCCAGA |
| | | CTCCCCTGTCCCTGCCTGTGACTCCCGGAGAACCAGCCTC |
| | | CATTTCCTGCCGGTCCTCCCAGTCCCTGCTGGACAGCGAC |
| | | GACGGCAACACTTACCTGGACTGGTACTTGCAGAAGCCG |
| | | GGCCAATCGCCTCAGCTGCTGATCTATACCCTGTCATTCC |
| | | GGGCCTCAGGAGTGCCTGACCGCTTCTCGGGATCAGGGA |
| | | GCGGGACCGATTTCACCCTGAAAATTAGGCGAGTGGAAG |
| | | CCGAGGACGTCGGAGTGTACTACTGCATGCAGCGCATCG |
| | | GCTTCCCGATTACGTTTGGACAGGGTACCCGGCTTGAGAT |
| | | CAAG |
| SEQ ID | Full CAR | EVQLVESGGGLVKPGGSLRLSCAASGFTFSSFRMNWVRQAP |
| NO: 177 | amino acid | GKGLEWVSSISSSSSYIYYADSVKGRFTISRDNAKNSLYLQM |
| | sequence | NSLRAEDTAVYYCARWLSYYGMDVWGQGTTVTVSSGGGG |
| | | SGGGGSGGGGSGGGGSDIVMTQTPLSLPVTPGEPASISCRSS |
| | | QSLLDSDDGNTYLDWYLQKPGQSPQLLIYTLSFRASGVPDR |
| | | FSGSGSGTDFTLKIRRVEAEDVGVYYCMQRIGFPITFGQGTR |
| | | LEIKTTTPAPRPPTPAPTIASQPLSLRPEACRPAAGGAVHTRG |
| | | LDFACDIYIWAPLAGTCGVLLLSLVITLYCKRGRKKLLYIFK |
| | | QPFMRPVQTTQEEDGCSCRFPEEEEGGCELRVKFSRSADAP |
| | | AYQQGQNQLYNELNLGRREEYDVLDKRRGRDPEMGGKPR |
| | | RKNPQEGLYNELQKDKMAEAYSEIGMKGERRRGKGHDGL |
| | | YQGLSTATKDTYDALHMQALPPR |
| SEQ ID | Full CAR | GAAGTGCAACTGGTGGAGAGCGGTGGAGGGCTTGTCAAG |
| NO: 178 | DNA | CCCGGAGGATCGCTGCGGCTGTCCTGTGCTGCGTCCGGGT |
| | sequence | TCACCTTCTCCTCGTTCCGCATGAACTGGGTCAGACAGGC |
| | | ACCGGGAAAGGGCCTCGAATGGGTGTCCTCAATCTCATC |
| | | GTCCTCGTCCTACATCTACTACGCCGACTCCGTGAAAGGC |
| | | CGCTTCACCATCTCCCGGGACAACGCCAAGAACTCGCTGT |
| | | ACCTCCAAATGAATAGCCTCAGGGCGGAAGATACTGCTG |
| | | TGTATTACTGCGCACGCTGGCTTTCCTACTACGGCATGGA |
| | | CGTCTGGGGCCAAGGGACCACTGTGACCGTGTCTAGCGG |
| | | AGGCGGAGGTTCAGGGGGCGGTGGATCAGGCGGAGGAG |
| | | GATCGGGGGGTGGTGGATCGGATATCGTGATGACCCAGA |
| | | CTCCCCTGTCCCTGCCTGTGACTCCCGGAGAACCAGCCTC |
| | | CATTTCCTGCCGGTCCTCCCAGTCCCTGCTGGACAGCGAC |
| | | GACGGCAACACTTACCTGGACTGGTACTTGCAGAAGCCG |
| | | GGCCAATCGCCTCAGCTGCTGATCTATACCCTGTCATTCC |
| | | GGGCCTCAGGAGTGCCTGACCGCTTCTCGGGATCAGGGA |
| | | GCGGGACCGATTTCACCCTGAAAATTAGGCGAGTGGAAG |
| | | CCGAGGACGTCGGAGTGTACTACTGCATGCAGCGCATCG |
| | | GCTTCCCGATTACGTTTGGACAGGGTACCCGGCTTGAGAT |
| | | CAAGACCACTACCCCAGCACCGAGGCCACCCACCCCGGC |
| | | TCCTACCATCGCCTCCCAGCCTCTGTCCCTGCGTCCGGAG |
| | | GCATGTAGACCCGCAGCTGGTGGGGCCGTGCATACCCGG |
| | | GGTCTTGACTTCGCCTGCGATATCTACATTTGGGCCCCTC |
| | | TGGCTGGTACTTGCGGGGTCCTGCTGCTTTCACTCGTGAT |
| | | CACTCTTTACTGTAAGCGCGGTCGGAAGAAGCTGCTGTAC |
| | | ATCTTTAAGCAACCCTTCATGAGGCCTGTGCAGACTACTC |
| | | AAGAGGAGGACGGCTGTTCATGCCGGTTCCCAGAGGAGG |
| | | AGGAAGGCGGCTGCGAACTGCGCGTGAAATTCAGCCGCA |
| | | GCGCAGATGCTCCAGCCTACCAGCAGGGGCAGAACCAGC |
| | | TCTACAACGAACTCAATCTTGGTCGGAGAGAGGAGTACG |
| | | ACGTGCTGGACAAGCGGAGAGGACGGGACCCAGAAATG |
| | | GGCGGGAAGCCGCGCAGAAAGAATCCCCAAGAGGGCCT |
| | | GTACAACGAGCTCCAAAAGGATAAGATGGCAGAAGCCTA |
| | | TAGCGAGATTGGTATGAAAGGGGAACGCAGAAGAGGCA |
| | | AAGGCCACGACGGACTGTACCAGGGACTCAGCACCGCCA |
| | | CCAAGGACACCTATGACGCTCTTCACATGCAGGCCCTGCC |
| | | GCCTCGG |

TABLE 11
Kabat CDRs of exemplary hybridoma-derived anti-BCMA molecules

| Kabat | HCDR1 | HCDR2 | HCDR3 | LCDR1 | LCDR2 | LCDR3 |
|---|---|---|---|---|---|---|
| Hy03 | GFWMS | NIKQDGSEK | ALDYYGMD | RSSQSLLD | TLSYRAS | TQRLEFP |
| | (SEQ ID | YYVDSVRG | V (SEQ | SDDGNTYL | (SEQ ID | SIT |
| | NO: 137) | (SEQ ID | ID NO: | D (SEQ | NO: 148) | (SEQ ID |
| | | NO: 138) | 139) | ID NO: | | NO: 149) |
| | | | | 147) | | |
| Hy52 | SFRMN | SISSSSSYI | WLSYYGMD | RSSQSLLD | TLSFRAS | MQRIGFP |
| | (SEQ ID | YYADSVKG | V (SEQ | SDDGNTYL | (SEQ ID | IT (SEQ |
| | NO: 160) | (SEQ ID | ID NO: | D (SEQ | NO: 170) | ID NO: |
| | | NO: 161) | 162) | ID NO: | | 171) |
| | | | | 147) | | |
| Con- | X₁FX₂MX₃, | X₁IX₂X₃X₄X₅ | X₁LX₂YYG | RSSQSLLD | TLSXRAS, | X₁QRX₂X₃ |
| sen- | wherein | SX₆X₇YYX₈ | MDV, | SDDGNTYL | wherein | FPX₄IT, |
| sus | X₁ is G | DSVX₉G, | wherein | D (SEQ | X is Y | wherein |
| | or S; X₂ | wherein X₁ | X₁ is A | ID NO: | or F | X₁ is T |
| | is W or | is N or S; | or W; | 147) | (SEQ ID | or M; X₂ |
| | R; and | X₂ is K or | and X₂ | | NO: 182) | is L or |
| | X₃ is S | S; X₃ is | is D or | | | I; X₃ is |
| | or N | Q or S; | S (SEQ | | | E or G; |
| | (SEQ ID | N or S; X₂ | ID NO: | | | and X₄ |
| | NO: 179) | is K or S; | 181) | | | is S or |
| | | X₃ is Q or | | | | absent |
| | | S; X₄ is D | | | | (SEQ ID |
| | | or S; X₅ | | | | NO: 183) |
| | | is G or S; | | | | |
| | | X₆ is E or | | | | |
| | | Y; X₇ is K | | | | |
| | | or I; X₈ is | | | | |
| | | V or A; | | | | |
| | | and X₉ is R | | | | |
| | | or K (SEQ | | | | |
| | | ID NO: 180) | | | | |

TABLE 12
Chothia CDRs of exemplary hybridoma-derived anti-BCMA molecules

| Chothia | HCDR1 | HCDR2 | HCDR3 | LCDR1 | LCDR2 | LCDR3 |
|---|---|---|---|---|---|---|
| Hy03 | GFTFSGF | KQDGSE | ALDYYGM | SQSLLDS | TLS | RLEFPSI |
| | (SEQ ID | (SEQ ID NO: | DV (SEQ ID | DDGNTY | (SEQ | (SEQ ID |
| | NO: 140) | 141) | NO: 139) | (SEQ ID | ID NO: | NO: 152) |
| | | | | NO: 150) | 151) | |
| Hy52 | GFTFSSF | SSSSSY (SEQ | WLSYYGM | SQSLLDS | TLS | RIGFPI |
| | (SEQ ID | ID NO: 164) | DV (SEQ ID | DDGNTY | (SEQ | (SEQ ID |
| | NO: 163) | | NO: 162) | (SEQ ID | ID NO: | NO: 172) |
| | | | | NO: 150) | 151) | |
| Consensus | GFTFSXF, | X₁X₂X₃X₄SX₅, | X₁LX₂YYG | SQSLLDS | TLS | RX₁X₂FP |
| | wherein X | wherein X₁ is | MDV, | DDGNTY | (SEQ | X₃I, |
| | is G or S | K or S; X₂ is Q | wherein X₁ is | (SEQ ID | ID NO: | wherein |
| | (SEQ ID | or S; X₃ is D or | A or W; and | NO: 150) | 151) | X₁ is L or |
| | NO: 184) | S; X₄ is G or S; | X₂ is D or S | | | I; X₂ is E |
| | | and X₅ is E or | (SEQ ID NO: | | | or G; and |
| | | Y (SEQ ID | 181) | | | X₃ is S or |
| | | NO: 185) | | | | absent |
| | | | | | | (SEQ ID |
| | | | | | | NO: 186) |

TABLE 13
IMGT CDRs of exemplary hybridoma-derived anti-BCMA molecules

| IMGT | HCDR1 | HCDR2 | HCDR3 | LCDR1 | LCDR2 | LCDR3 |
|---|---|---|---|---|---|---|
| Hy03 | GFTFSGF | IKQDGSEK | ARALDYY | QSLLDSD | TLS | TQRLEFP |
| | W (SEQ ID | (SEQ ID | GMDV | DGNTY | (SEQ ID | SIT (SEQ |
| | NO: 142) | NO: 143) | (SEQ ID | (SEQ ID | NO: | ID NO: |
| | | | NO: 144) | NO: 153) | 151) | 149) |
| Hy52 | GFTFSSF | ISSSSSYI | ARWLSYY | QSLLDSD | TLS | MQRIGFP |
| | R (SEQ ID | (SEQ ID | GMDV | DGNTY | (SEQ ID | IT (SEQ |
| | NO: 165) | NO: 166) | (SEQ ID | (SEQ ID | NO: | ID NO: |
| | | | NO: 167) | NO: 153) | 151) | 171) |
| Consensus | GFTFSX₁F | IX₁X₂X₃X₄S | ARX₁LX₂Y | QSLLDSD | TLS | X₁QRX₂X₃ |
| | X₂, | X₅X₆, | YGMDV, | DGNTY | (SEQ ID | FPX₄IT, |
| | wherein X₁ | wherein X₁ is | wherein X₁ is | (SEQ ID | NO: | wherein X₁ |
| | is G or S; | K or S; X₂ is | A or W; and | NO: 153) | 151) | is T or M; |
| | and X₂ is | Q or S; X₃ is | X₂ is D or S | | | X₂ is L or |
| | W or R | D or S; X₄ is | (SEQ ID | | | I; X₃ is E |
| | (SEQ ID | G or S; X₅ is | NO: 189) | | | or G; and |
| | NO: 187) | E or Y; and | | | | X₄ is S or |
| | | X₆ is K or I | | | | absent |
| | | (SEQ ID | | | | (SEQ ID |
| | | NO: 188) | | | | NO: 183) |

In some embodiments, BCMA CARs may be generated using the VH and VL sequences from WO2012/0163805 (the contents of which are hereby incorporated by reference in its entirety).

In some embodiments, BCMA CARs may be generated using the CDRs, VHs, VLs, scFvs, or full-CAR sequences from WO2019/241426 (the contents of which are hereby incorporated by reference in its entirety).

### Other Exemplary Targets

Further non-limiting exemplary tumor antigens include CD20, CD22, EGFR, CD123, and CLL-1.

CARs that bind to CD20 are known in the art. For example, those disclosed in WO2018/067992 or WO2016/164731, incorporated by reference herein. Any known CD20 CAR, for example, the CD20 antigen binding domain of any known CD20 CAR, in the art can be used in accordance with the present disclosure. Exemplary CD20-binding sequences or CD20 CAR sequences are disclosed in, for example, Tables 1-5 of WO2018/067992, incorporated by reference. In some embodiments, the CD20 CAR comprises a CDR, variable region, scFv, or full-length sequence of a CD20 CAR disclosed in WO2018/067992 or WO2016/164731, both incorporated by reference herein.

CARs that bind to CD22 are known in the art. For example, those disclosed in WO2018/067992 or WO2016/164731. Any known CD22 CAR, for example, the CD22 antigen binding domain of any known CD22 CAR, in the art can be used in accordance with the present disclosure.

Exemplary CD22-binding sequences or CD22 CAR sequences are disclosed in, for example, Tables 6A, 6B, 7A, 7B, 7C, 8A, 8B, 9A, 9B, 10A, and 10B of WO2016164731 and Tables 6-10 of WO2018067992. In some embodiments, the CD22 CAR sequences comprise a CDR, variable region, scFv or full-length sequence of a CD22 CAR disclosed in WO2018067992 or WO2016164731.

In embodiments, the CAR comprises an antigen binding domain that binds to CD22 (CD22 CAR). In some embodiments, the antigen binding domain targets human CD22. In some embodiments, the antigen binding domain includes a single chain Fv sequence as described herein.

The sequences of human CD22 CAR are provided below. In some embodiments, a human CD22 CAR is CAR22-65.

Human CD22 CAR scFv sequence
(SEQ ID NO: 671)
EVQLQQSGPGLVKPSQTLSLTCAISGDSMLSNSDTWNWIRQSPSRGLE
WLGRTYHRSTWYDDYASSVRGRVSINVDTSKNQYSLQLNAVTPEDTGV
YYCARVRLQDGNSWSDAFDVWGQGTMVTVSSGGGGSGGGGSGGGGSQS
ALTQPASASGSPGQSVTISCTGTSSDVGGYNYVSWYQQHPGKAPKLMI
YDVSNRPSGVSNRFSGSKSGNTASLTISGLQAEDEADYYCSSYTSSST
LYVFGTGTQLTVL
Human CD22 CAR heavy chain variable region
(SEQ ID NO 672)
EVQLQQSGPGLVKPSQTLSLTCAISGDSMLSNSDTWNWIRQSPSRGLE
WLGRTYHRSTWYDDYASSVRGRVSINVDTSKNQYSLQLNAVTPEDTGV
YYCARVRLQDGNSWSDAFDVWGQGTMVTVSS
Human CD22 CAR light chain variable region
(SEQ ID NO 673)
QSALTQPASASGSPGQSVTISCTGTSSDVGGYNYVSWYQQHPGKAPKL
MIYDVSNRPSGVSNRFSGSKSGNTASLTISGLQAEDEADYYCSSYTSS
STLYVFGTGTQLTVL

TABLE 15
Heavy Chain Variable Domain CDRs of CD22 CAR (CAR22-65)

| Candidate | HCDR1 | SEQ ID NO: | HCDR2 | SEQ ID NO: | HCDR3 | SEQ ID NO: |
|---|---|---|---|---|---|---|
| CAR22-65 | GDSMLSNS | 498 | RTYHRST | 509 | VRLQDGN | 520 |
| Combined | DTWN | | WYDDYAS | | SWSDAFD | |
| | | | SVRG | | V | |
| CAR22-65 | SNSDTWN | 499 | RTYHRST | 510 | VRLQDGN | 521 |
| Kabat | | | WYDDYAS | | SWSDAFD | |
| | | | SVRG | | V | |

TABLE 16
Light Chain Variable Domain CDRs of CD22 CAR (CAR22-65). The
LC CDR sequences in this table have the same sequence
under the Kabat or combined definitions.

| Candidate | LCDR1 | SEQ ID NO: | LCDR2 | SEQ ID NO: | LCDR3 | SEQ ID NO: |
|---|---|---|---|---|---|---|
| CAR22-65 | TGTSSDVG | 648 | DVSNRPS | 659 | SSYTSSSTL | 670 |
| Combined | GYNYVS | | | | YV | |

CARs that bind to EGFR are known in the art. For example, those disclosed in WO2014/130657, incorporated by reference herein. Any known EGFR CAR, for example, the EGFR antigen binding domain of any known EGFR CAR, in the art can be used in accordance with the present disclosure. Exemplary EGFRvIII CARs can include a CDR, a variable region, an scFv, or a full-length CAR sequence disclosed in WO2014/130657, for example, Table 2 of WO2014/130657, incorporated herein by reference.

CARs that bind to CD123 are known in the art. For example, those disclosed in WO2014/130635 or WO2016/028896. Any known CD123 CAR, for example, the CD123 antigen binding domain of any known CD123 CAR, in the art can be used in accordance with the present disclosure. For example, CAR1 to CAR8 disclosed in WO2014/130635; or CAR123-1 to CAR123-4 and hzCAR123-1 to hzCAR123-32, disclosed in WO2016/028896. The amino acid and nucleotide sequences encoding the CD123 CAR molecules and antigen binding domains (for example, including one, two, three VH CDRs; and one, two, three VL CDRs according to Kabat or Chothia), are specified in WO 2014/130635 and WO2016/028896.

CARs that bind to CLL-1 are known in the art. For example, those disclosed in US2016/0051651A1, incorporated herein by reference. Any known CLL-1 CAR, for example, the CLL-1 antigen binding domain of any known CLL-1 CAR, in the art can be used in accordance with the present disclosure.

In some embodiments, the CAR comprises a CLL-1 CAR or antigen binding domain according to Table 2 of WO2016/014535, incorporated herein by reference. The amino acid and nucleotide sequences encoding the CLL-1 CAR molecules and antigen binding domains (for example, including one, two, three VH CDRs; and one, two, three VL CDRs according to Kabat or Chothia), are specified in WO2016/014535.

CARs that bind to CD33 are known in the art. For example, those disclosed in US2016/0096892A1 and WO2016/014576, incorporated by reference herein. Any known CD33 CAR, for example, the CD33 antigen binding domain of any known CD33 CAR, in the art can be used in accordance with the present disclosure. For example, CAR33-1 to CAR33-9 disclosed in WO2016/014576.

In some embodiments, the CAR comprises a CD33 CAR or antigen binding domain according to Table 2 or 9 of WO2016/014576, incorporated herein by reference. The amino acid and nucleotide sequences encoding the CD33 CAR molecules and antigen binding domains (for example, including one, two, three VH CDRs; and one, two, three VL CDRs according to Kabat or Chothia), are specified in WO2016/014576.

CARs that bind to mesothelin are known in the art. For example, those disclosed in WO2015090230 and WO2017112741, for example, Tables 2, 3, 4, and 5 of WO2017112741, incorporated herein by reference, that bind human mesothelin. Any known mesothelin CAR, for example, the mesothelin antigen binding domain of any known mesothelin CAR, in the art can be used in accordance with the present disclosure.

CARs that bind to GFR ALPHA-4 are known in the art. For example, those disclosed in WO2016/025880. Any known GFR ALPHA-4 CAR, for example, the GFR ALPHA-4 antigen binding domain of any known GFR ALPHA-4 CAR, in the art can be used in accordance with the present disclosure. The amino acid and nucleotide sequences encoding the GFR ALPHA-4 CAR molecules and antigen binding domains (for example, including one, two, three VH CDRs; and one, two, three VL CDRs according to Kabat or Chothia), are specified in WO2016/025880.

Antigen Binding Domain Structures

In some embodiments, the antigen binding domain of the encoded CAR molecule comprises an antibody, an antibody fragment, an scFv, a Fv, a Fab, a (Fab′)2, a single domain antibody (SDAB), a VH or VL domain, a camelid VHH domain or a bi-functional (e.g. bi-specific) hybrid antibody (e.g., Lanzavecchia et al., Eur. J. Immunol. 17, 105 (1987)).

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site.

For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly4Ser)n, where n is a positive integer equal to or greater than 1 (SEQ ID NO:22). In some embodiments, the linker can be (Gly4Ser)4 (SEQ ID NO:29) or (Gly4Ser)3 (SEQ ID NO:30). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellar, however, a fragment of such antigen (peptide) is presented on the surface of the cancer cells by MHC.

In certain embodiments, the encoded antigen binding domain has a binding affinity KD of 10⁻⁴M to 10⁻⁸M.

In some embodiments, the encoded CAR molecule comprises an antigen binding domain that has a binding affinity KD of 10⁻⁴M to 10⁻⁸M, e.g., 10⁻⁵ M to 10⁻⁷ M, e.g., 10⁻⁶M or 10⁻⁷ M, for the target antigen. In some embodiments, the antigen binding domain has a binding affinity that is at least five-fold, 10-fold, 20-fold, 30-fold, 50-fold, 100-fold or 1,000-fold less than a reference antibody, e.g., an antibody described herein. In some embodiments, the encoded antigen binding domain has a binding affinity at least 5-fold less than a reference antibody (e.g., an antibody from which the antigen binding domain is derived). In some aspects such antibody fragments are functional in that they provide a biological response that can include, but is not limited to, activation of an immune response, inhibition of signal-transduction origination from its target antigen, inhibition of kinase activity, and the like, as will be understood by a skilled artisan.

In some aspects, the antigen binding domain of the CAR is a scFv antibody fragment that is humanized compared to the murine sequence of the scFv from which it is derived.

In some aspects, the antigen binding domain of a CAR described herein (e.g., a scFv) is encoded by a nucleic acid molecule whose sequence has been codon optimized for expression in a mammalian cell. In some aspects, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

Specific antigen antibody pairs are known in the art. Non-limiting exemplary embodiments of antigen antibody pairs and components thereof are provided herein above in the section titled Targets and below.

CD19

In some embodiments, the antigen binding domain binds to CD19 and has the same or a similar binding specificity as the FMC63 scFv fragment described in Nicholson et al. Mol. Immun. 34 (16-17): 1157-1165 (1997). In some embodiments, the antigen binding domain binds to CD19 and includes the scFv fragment described in Nicholson et al. Mol. Immun. 34 (16-17): 1157-1165 (1997).

In some embodiments, the antigen binding domain (for example, a humanized antigen binding domain) binds to CD19 and comprises a sequence from Table 3 of WO2014/153270, incorporated herein by reference. WO2014/153270 also describes methods of assaying the binding and efficacy of various CAR constructs.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, the antigen binding domain comprises the parental murine scFv sequence of the CAR19 construct provided in WO2012/079000 (incorporated herein by reference). In some embodiments, the antigen binding domain binds CD19 and comprises a scFv described in WO2012/079000.

BCMA

Exemplary antigen binding domains that bind BCMA are disclosed in WO2012/0163805, WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, the antigen binding domain of one or more of the BCMA antigen binding domains disclosed therein.

In some embodiments, the antigen binding domain comprises a human antibody or a human antibody fragment that binds BCMA. In some embodiments, the antigen binding domain comprises one or more (for example, all three) LC CDR1, LC CDR2, and LC CDR3 of a human anti-BCMA binding domain described herein (for example, in Tables 2-14), and/or one or more (for example, all three) HC CDR1, HC CDR2, and HC CDR3 of a human anti-BCMA binding domain described herein (for example, in Tables 2-14). In some embodiments, the human anti-BCMA binding domain comprises a human VL described herein (for example, in Tables 2, 6, and 10) and/or a human VH described herein (for example, in Tables 2, 6, and 10). In some embodiments, the antigen binding domain is a scFv comprising a VL and a VH of an amino acid sequence of Tables 2, 6, and 10. In some embodiments, the antigen binding domain (for example, an scFv) comprises: a VL comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 2, 6, and 10, or a sequence with 95-99% identity with an amino acid sequence of Tables 2, 6, and 10; and/or a VH comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 2, 6, and 10, or a sequence with 95-99% identity to an amino acid sequence of Tables 2, 6, and 10.

In certain embodiments, the antigen binding domain described herein includes:

(1) one, two, or three light chain (LC) CDRs chosen from:

(i) a LC CDR1 of SEQ ID NO: 54, LC CDR2 of SEQ ID NO: 55 and LC CDR3 of SEQ ID NO: 56; and/or

(2) one, two, or three heavy chain (HC) CDRs from one of the following:

(i) a HC CDR1 of SEQ ID NO: 44, HC CDR2 of SEQ ID NO: 45 and HC CDR3 of SEQ ID NO: 84; (ii) a HC CDR1 of SEQ ID NO: 44, HC CDR2 of SEQ ID NO: 45 and HC CDR3 of SEQ ID NO: 46; (iii) a HC CDR1 of SEQ ID NO: 44, HC CDR2 of SEQ ID NO: 45 and HC CDR3 of SEQ ID NO: 68; or (iv) a HC CDR1 of SEQ ID NO: 44, HC CDR2 of SEQ ID NO: 45 and HC CDR3 of SEQ ID NO: 76.

In certain embodiments, the antigen binding domain described herein includes:

(1) one, two, or three light chain (LC) CDRs from one of the following:

(i) a LC CDR1 of SEQ ID NO: 95, LC CDR2 of SEQ ID NO: 131 and LC CDR3 of SEQ ID NO: 132; (ii) a LC CDR1 of SEQ ID NO: 95, LC CDR2 of SEQ ID NO: 96 and LC CDR3 of

SEQ ID NO: 97; (iii) a LC CDR1 of SEQ ID NO: 95, LC CDR2 of SEQ ID NO: 114 and LC CDR3 of SEQ ID NO: 115; or (iv) a LC CDR1 of SEQ ID NO: 95, LC CDR2 of SEQ ID NO: 114 and LC CDR3 of SEQ ID NO: 97; and/or

(2) one, two, or three heavy chain (HC) CDRs from one of the following:

(i) a HC CDR1 of SEQ ID NO: 86, HC CDR2 of SEQ ID NO: 130 and HC CDR3 of SEQ ID NO: 88; (ii) a HC CDR1 of SEQ ID NO: 86, HC CDR2 of SEQ ID NO: 87 and HC CDR3 of SEQ ID NO: 88; or (iii) a HC CDR1 of SEQ ID NO: 86, HC CDR2 of SEQ ID NO: 109 and HC CDR3 of SEQ ID NO: 88.

In certain embodiments, the antigen binding domain described herein includes:

(1) one, two, or three light chain (LC) CDRs from one of the following:

(i) a LC CDR1 of SEQ ID NO: 147, LC CDR2 of SEQ ID NO: 182 and LC CDR3 of SEQ ID NO: 183; (ii) a LC CDR1 of SEQ ID NO: 147, LC CDR2 of SEQ ID NO: 148 and LC CDR3 of SEQ ID NO: 149; or (iii) a LC CDR1 of SEQ ID NO: 147, LC CDR2 of SEQ ID NO: 170 and LC CDR3 of SEQ ID NO: 171; and/or

(2) one, two, or three heavy chain (HC) CDRs from one of the following:

(i) a HC CDR1 of SEQ ID NO: 179, HC CDR2 of SEQ ID NO: 180 and HC CDR3 of SEQ ID NO: 181; (ii) a HC CDR1 of SEQ ID NO: 137, HC CDR2 of SEQ ID NO: 138 and HC CDR3 of SEQ ID NO: 139; or (iii) a HC CDR1 of SEQ ID NO: 160, HC CDR2 of SEQ ID NO: 161 and HC CDR3 of SEQ ID NO: 162.

In some embodiments, the HC CDR1, HC CDR2, HC CDR3, LC CDR1, LC CDR2, and LC CDR3 comprise the amino acid sequences of SEQ ID NOs: 44, 45, 84, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 46, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 68, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 76, 54, 55, and 56, respectively.

In some embodiments, the HC CDR1, HC CDR2, HC CDR3, LC CDR1, LC CDR2, and LC CDR3 comprise the amino acid sequences of SEQ ID NOs: 47, 48, 84, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 46, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 68, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 76, 57, 58, and 59, respectively.

In some embodiments, the HC CDR1, HC CDR2, HC CDR3, LC CDR1, LC CDR2, and LC CDR3 comprise the amino acid sequences of SEQ ID NOs: 49, 50, 85, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 51, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 69, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 77, 60, 58, and 56, respectively.

Other Exemplary Targets

Exemplary antigen binding domains that bind CD20 are described in WO2016/164731 and WO2018/067992, incorporated herein by reference. In some embodiments, the antigen binding domain of one or more of the CD20 antigen binding domains disclosed therein.

Exemplary antigen binding domains that bind CD22 are described in WO2016/164731 and WO2018/067992, incorporated herein by reference.

In some embodiments, the antigen binding domain comprises a HC CDR1, a HC CDR2, and a HC CDR3 of any heavy chain binding domain amino acid sequences listed in Table 15. In embodiments, the antigen binding domain further comprises a LC CDR1, a LC CDR2, and a LC CDR3. In embodiments, the antigen binding domain comprises a LC CDR1, a LC CDR2, and a LC CDR3 amino acid sequences listed in Table 16.

In some embodiments, the antigen binding domain comprises one, two or all of LC CDR1, LC CDR2, and LC CDR3 of any light chain binding domain amino acid sequences listed in Table 16, and one, two or all of HC CDR1, HC CDR2, and HC CDR3 of any heavy chain binding domain amino acid sequences listed in Table 15.

Exemplary antigen binding domains that bind EGFRvIII are described in in WO2014/130657.

Exemplary antigen binding domains that bind CD123 are described in WO 2014/130635 and WO2016/028896, incorporated herein by reference.

In some embodiments, the antigen binding domain comprises a sequence from Tables 1-2 of WO2014/130635, incorporated herein by reference.

In some embodiments, the antigen binding domain comprises a sequence from Tables 2, 6, and 9 of WO2016/028896, incorporated herein by reference.

Exemplary antigen binding domains that bind CLL-1 are disclosed in WO2016/014535, incorporated herein by reference.

In some embodiments, the antigen binding domain comprises one, two three (for example, all three) heavy chain CDRs, HC CDR1, HC CDR2 and HC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (for example, all three) light chain CDRs, LC CDR1, LC CDR2 and LC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In some embodiments, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In some embodiments, the antigen binding domain of any of the CARs described herein (for example, any of CD19, CD123, EGFRvIII, CD33, mesothelin, BCMA, and GFR ALPHA-4) comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antigen binding domain listed above. In some embodiments, the antigen binding domain comprises a heavy chain variable region and/or a variable light chain region of an antibody listed or described above.

In some embodiments, the antigen binding domain comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

Bispecific CARs

In certain embodiments, the antigen binding domain is a bi- or multi-specific molecule (e.g., a multispecific antibody molecule). In some embodiments a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments the first and second epitopes are on the same antigen, e.g., the same protein (or subunit of a multimeric protein). In some embodiments the first and second epitopes overlap. In some embodiments the first and second epitopes do not overlap. In some embodiments the first and second epitopes are on different antigens, e.g., different proteins (or different subunits of a multimeric protein). In some embodiments a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments a bispecific antibody molecule comprises a half antibody having binding specificity for a first epitope and a half antibody having binding specificity for a second epitope. In some embodiments a bispecific antibody molecule comprises a half antibody, or fragment thereof, having binding specificity for a first epitope and a half antibody, or fragment thereof, having binding specificity for a second epitope. In some embodiments a bispecific antibody molecule comprises a scFv, or fragment thereof, have binding specificity for a first epitope and a scFv, or fragment thereof, have binding specificity for a second epitope.

In some embodiments, the antibody molecule is a multi-specific (e.g., a bispecific or a trispecific) antibody molecule. Such molecules include bispecific fusion proteins, e.g., an expression construct containing two scFvs with a hydrophilic helical peptide linker between them and a full constant region, as described in, e.g., U.S. Pat. No. 5,637,481; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH1) upstream of its VL (VL1) and the downstream antibody or antibody fragment (e.g., scFv) is arranged with its VL (VL2) upstream of its VH (VH2), such that the overall bispecific antibody molecule has the arrangement VH1-VL1-VL2-VH2. In other embodiments, the upstream antibody or antibody fragment (e.g., scFv) is arranged with its VL (VL1) upstream of its VH (VH1) and the downstream antibody or antibody fragment (e.g., scFv) is arranged with its VH (VH2) upstream of its VL (VL2), such that the overall bispecific antibody molecule has the arrangement VL1-VH1-VH2-VL2. Optionally, a linker is disposed between the two antibodies or antibody fragments (e.g., scFvs), e.g., between VL1 and VL2 if the construct is arranged as VH1-VL1-VL2-VH2, or between VH1 and VH2 if the construct is arranged as VL1-VH1-VH2-VL2. The linker may be a linker as described herein, e.g., a (Gly4-Ser)n linker, wherein n is 1, 2, 3, 4, 5, or 6, preferably 4 (SEQ ID NO: 691). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

1. Transmembrane Domains

With respect to the transmembrane domain, in various embodiments, a chimeric molecule as described herein (e.g., a CAR) can be designed to comprise a transmembrane domain that is attached to the extracellular domain of the chimeric molecule. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some aspects, the transmembrane domain is one that is associated with one of the other domains of the chimeric protein (e.g., CAR) e.g., in some embodiments, the transmembrane domain may be from the same protein that the signaling domain, costimulatory domain or the hinge domain is derived from. In another aspect, the transmembrane domain is not derived from the same protein that any other domain of the chimeric protein (e.g., CAR) is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some aspects, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In some aspects the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C.

In some instances, the transmembrane domain can be attached to the extracellular region of the CAR, e.g., the antigen binding domain of the CAR, via a hinge, e.g., a hinge from a human protein. For example, in some embodiments, the hinge can be a human Ig (immunoglobulin) hinge (e.g., an IgG4 hinge, an IgD hinge), a GS linker (e.g., a GS linker described herein), a KIR2DS2 hinge or a CD8a hinge. In some embodiments, the hinge or spacer comprises (e.g., consists of) the amino acid sequence of SEQ ID NO:4. In some aspects, the transmembrane domain comprises (e.g., consists of) a transmembrane domain of SEQ ID NO: 12.

In some embodiments, the encoded transmembrane domain comprises an amino acid sequence of a CD8 transmembrane domain having at least one, two or three modifications but not more than 20, 10 or 5 modifications of an amino acid sequence of SEQ ID NO: 12, or a sequence with 95-99% identity to an amino acid sequence of SEQ ID NO: 12. In some embodiments, the encoded transmembrane domain comprises the sequence of SEQ ID NO: 12.

In other embodiments, the nucleic acid molecule encoding the CAR comprises a nucleotide sequence of a CD8 transmembrane domain, e.g., comprising the sequence of SEQ ID NO: 13, or a sequence with 95-99% identity thereof.

In some embodiments, the encoded antigen binding domain is connected to the transmembrane domain by a hinge region. In some embodiments, the encoded hinge region comprises the amino acid sequence of a CD8 hinge, e.g., SEQ ID NO: 4; or the amino acid sequence of an IgG4 hinge, e.g., SEQ ID NO: 6, or a sequence with 95-99% identity to SEQ ID NO:4 or 6. In other embodiments, the nucleic acid sequence encoding the hinge region comprises a sequence of SEQ ID NO: 5 or SEQ ID NO: 7, corresponding to a CD8 hinge or an IgG4 hinge, respectively, or a sequence with 95-99% identity to SEQ ID NO:5 or 7.

In some aspects, the hinge or spacer comprises an IgG4 hinge. For example, in some embodiments, the hinge or spacer comprises a hinge of the amino acid sequence ESKYGPPCPPCPAPEFLGGPSVFLFPPKPKDTLMISRTPEVTCVVVDVSQEDPEVQFNWY VDGVEVHNAKTKPREEQFNSTYRVVSVLTVLHQDWLNGKEYKCKVSNKGLPSSIEKTIS KAKGQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGKM (SEQ ID NO:6). In some embodiments, the hinge or spacer comprises a hinge encoded by a nucleotide sequence of GAGAGCAAGTACGGCCCTCCCTGCCCCCCTTGCCCTGCCCCCGAGTTCCTGGGCGGA CCCAGCGTGTTCCTGTTCCCCCCCAAGCCCAAGGACACCCTGATGATCAGCCGGACC CCCGAGGTGACCTGTGTGGTGGTGGACGTGTCCCAGGAGGACCCCGAGGTCCAGTT CAACTGGTACGTGGACGGCGTGGAGGTGCACAACGCCAAGACCAAGCCCCGGGAG GAGCAGTTCAATAGCACCTACCGGGTGGTGTCCGTGCTGACCGTGCTGCACCAGGA CTGGCTGAACGGCAAGGAATACAAGTGTAAGGTGTCCAACAAGGGCCTGCCCAGCA GCATCGAGAAAACCATCAGCAAGGCCAAGGGCCAGCCTCGGGAGCCCCAGGTGTAC ACCCTGCCCCCTAGCCAAGAGGAGATGACCAAGAACCAGGTGTCCCTGACCTGCCT GGTGAAGGGCTTCTACCCCAGCGACATCGCCGTGGAGTGGGAGAGCAACGGCCAGC CCGAGAACAACTACAAGACCACCCCCCCTGTGCTGGACAGCGACGGCAGCTTCTTC CTGTACAGCCGGCTGACCGTGGACAAGAGCCGGTGGCAGGAGGGCAACGTCTTTAG CTGCTCCGTGATGCACGAGGCCCTGCACAACCACTACACCCAGAAGAGCCTGAGCC TGTCCCTGGGCAAGATG (SEQ ID NO:7).

In some aspects, the hinge or spacer comprises an IgD hinge. For example, in some embodiments, the hinge or spacer comprises a hinge of the amino acid sequence RWPESPKAQASSVPTAQPQAEGSLAKATTAPATTRNTGRGGEEKKKEKEKEEQEERETK TPECPSHTQPLGVYLLTPAVQDLWLRDKATFTCFVVGSDLKDAHLTWEVAGKVPTGGV EEGLLERHSNGSQSQHSRLTLPRSLWNAGTSVTCTLNHPSLPPQRLMALREPAAQAPVK LSLNLLASSDPPEAASWLLCEVSGFSPPNILLMWLEDQREVNTSGFAPARPPPQPGSTTF WAWSVLRVPAPPSPQPATYTCVVSHEDSRTLLNASRSLEVSYVTDH (SEQ ID NO:8). In some embodiments, the hinge or spacer comprises a hinge encoded by a nucleotide sequence of AGGTGGCCCGAAAGTCCCAAGGCCCAGGCATCTAGTGTTCCTACTGCACAGCCCCA GGCAGAAGGCAGCCTAGCCAAAGCTACTACTGCACCTGCCACTACGCGCAATACTG GCCGTGGCGGGGAGGAGAAGAAAAAGGAGAAAGAGAAAGAAGAACAGGAAGAGA GGGAGACCAAGACCCCTGAATGTCCATCCCATACCCAGCCGCTGGGCGTCTATCTCT TGACTCCCGCAGTACAGGACTTGTGGCTTAGAGATAAGGCCACCTTTACATGTTTCG TCGTGGGCTCTGACCTGAAGGATGCCCATTTGACTTGGGAGGTTGCCGGAAAGGTAC CCACAGGGGGGGTTGAGGAAGGGTTGCTGGAGCGCCATTCCAATGGCTCTCAGAGC CAGCACTCAAGACTCACCCTTCCGAGATCCCTGTGGAACGCCGGGACCTCTGTCACA TGTACTCTAAATCATCCTAGCCTGCCCCCACAGCGTCTGATGGCCCTTAGAGAGCCA GCCGCCCAGGCACCAGTTAAGCTTAGCCTGAATCTGCTCGCCAGTAGTGATCCCCCA GAGGCCGCCAGCTGGCTCTTATGCGAAGTGTCCGGCTTTAGCCCGCCCAACATCTTG CTCATGTGGCTGGAGGACCAGCGAGAAGTGAACACCAGCGGCTTCGCTCCAGCCCG GCCCCCACCCCAGCCGGGTTCTACCACATTCTGGGCCTGGAGTGTCTTAAGGGTCCC AGCACCACCTAGCCCCCAGCCAGCCACATACACCTGTGTTGTGTCCCATGAAGATAG CAGGACCCTGCTAAATGCTTCTAGGAGTCTGGAGGTTTCCTACGTGACTGACCATT (SEQ ID NO:9).

In some aspects, the transmembrane domain may be recombinant, in which case it will comprise predominantly hydrophobic residues such as leucine and valine. In some aspects a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in some aspects, the linker comprises the amino acid sequence of GGGGSGGGGS (SEQ ID NO:10). In some embodiments, the linker is encoded by a nucleotide sequence of

(SEQ ID NO: 11)
GGTGGCGGAGGTTCTGGAGGTGGAGGTTCC.

In some aspects, the hinge or spacer comprises a KIR2DS2 hinge.

2. Signaling Domains

In embodiments of the invention having an intracellular signaling domain, such a domain can contain, e.g., one or more of a primary signaling domain and/or a costimulatory signaling domain. In some embodiments, the intracellular signaling domain comprises a sequence encoding a primary signaling domain. In some embodiments, the intracellular signaling domain comprises a costimulatory signaling domain. In some embodiments, the intracellular signaling domain comprises a primary signaling domain and a costimulatory signaling domain.

The intracellular signaling sequences within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In some embodiments, a glycine-serine doublet can be used as a suitable linker. In some embodiments, a single amino acid, e.g., an alanine, a glycine, can be used as a suitable linker.

In some aspects, the intracellular signaling domain is designed to comprise two or more, e.g., 2, 3, 4, 5, or more, costimulatory signaling domains. In some embodiments, the two or more, e.g., 2, 3, 4, 5, or more, costimulatory signaling domains, are separated by a linker molecule, e.g., a linker molecule described herein. In some embodiments, the intracellular signaling domain comprises two costimulatory signaling domains. In some embodiments, the linker molecule is a glycine residue. In some embodiments, the linker is an alanine residue.

Primary Signaling Domains

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. In CARs such domains are used for the same purpose.

Examples of ITAM containing primary intracellular signaling domains that are of particular use in the invention include those of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In some embodiments, a CAR of the invention comprises an intracellular signaling domain, e.g., a primary signaling domain of CD3-zeta.

In some embodiments, the encoded primary signaling domain comprises a functional signaling domain of CD3 zeta. The encoded CD3 zeta primary signaling domain can comprise an amino acid sequence having at least one, two or three modifications but not more than 20, 10 or 5 modifications of an amino acid sequence of SEQ ID NO: 18 or SEQ ID NO: 20, or a sequence with 95-99% identity to an amino acid sequence of SEQ ID NO:18 or SEQ ID NO: 20. In some embodiments, the encoded primary signaling domain comprises a sequence of SEQ ID NO:18 or SEQ ID NO: 20. In other embodiments, the nucleic acid sequence encoding the primary signaling domain comprises a sequence of SEQ ID NO:19 or SEQ ID NO: 21, or a sequence with 95-99% identity thereof.

Costimulatory Signaling Domains

In some embodiments, the encoded intracellular signaling domain comprises a costimulatory signaling domain. For example, the intracellular signaling domain can comprise a primary signaling domain and a costimulatory signaling domain. In some embodiments, the encoded costimulatory signaling domain comprises a functional signaling domain of a protein chosen from one or more of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the encoded costimulatory signaling domain comprises an amino acid sequence having at least one, two or three modifications but not more than 20, 10 or 5 modifications of an amino acid sequence of SEQ ID NO:14 or SEQ ID NO: 16, or a sequence with 95-99% identity to an amino acid sequence of SEQ ID NO:14 or SEQ ID NO: 16. In some embodiments, the encoded costimulatory signaling domain comprises a sequence of SEQ ID NO: 14 or SEQ ID NO: 16. In other embodiments, the nucleic acid sequence encoding the costimulatory signaling domain comprises a sequence of SEQ ID NO:15 or SEQ ID NO: 17, or a sequence with 95-99% identity thereof.

In other embodiments, the encoded intracellular domain comprises the sequence of SEQ ID NO: 14 or SEQ ID NO: 16, and the sequence of SEQ ID NO: 18 or SEQ ID NO: 20, wherein the sequences comprising the intracellular signaling domain are expressed in the same frame and as a single polypeptide chain.

In some embodiments, the nucleic acid sequence encoding the intracellular signaling domain comprises a sequence of SEQ ID NO:15 or SEQ ID NO: 17, or a sequence with 95-99% identity thereof, and a sequence of SEQ ID NO:19 or SEQ ID NO:21, or a sequence with 95-99% identity thereof.

In some embodiments, the nucleic acid molecule further encodes a leader sequence. In some embodiments, the leader sequence comprises the sequence of SEQ ID NO: 2.

In some aspects, the intracellular signaling domain is designed to comprise the signaling domain of CD3-zeta and the signaling domain of CD28. In some aspects, the intracellular signaling domain is designed to comprise the signaling domain of CD3-zeta and the signaling domain of 4-1BB. In some aspects, the signaling domain of 4-1BB is a signaling domain of SEQ ID NO: 14. In some aspects, the signaling domain of CD3-zeta is a signaling domain of SEQ ID NO: 18.

In some aspects, the intracellular signaling domain is designed to comprise the signaling domain of CD3-zeta and the signaling domain of CD27. In some aspects, the signaling domain of CD27 comprises an amino acid sequence of QRRKYRSNKGESPVEPAEPCRYSCPREEEGSTIPIQEDYRKPEPACSP (SEQ ID NO:16). In some aspects, the signaling domain of CD27 is encoded by a nucleic acid sequence of

(SEQ ID NO: 17)
AGGAGTAAGAGGAGCAGGCTCCTGCACAGTGACTACATGAACATGACT
CCCCGCCGCCCCGGGCCCACCCGCAAGCATTACCAGCCCTATGCCCCA
CCACGCGACTTCGCAGCCTATCGCTCC.

Inhibitory Domains

In some embodiments, the vector comprises a nucleic acid sequence that encodes a CAR, e.g., a CAR described herein, and a nucleic acid sequence that encodes an inhibitory molecule comprising: an inhKIR cytoplasmic domain; a transmembrane domain, e.g., a KIR transmembrane domain; and an inhibitor cytoplasmic domain, e.g., an ITIM domain, e.g., an inhKIR ITIM domain. In some embodiments the inhibitory molecule is a naturally occurring inhKIR, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring inhKIR.

In some embodiments, the nucleic acid sequence that encodes an inhibitory molecule comprises: a SLAM family cytoplasmic domain; a transmembrane domain, e.g., a SLAM family transmembrane domain; and an inhibitor cytoplasmic domain, e.g., a SLAM family domain, e.g., an SLAM family ITIM domain. In some embodiments the inhibitory molecule is a naturally occurring SLAM family member, or a sequence sharing at least 50, 60, 70, 80, 85, 90, 95, or 99% homology with, or that differs by no more than 1, 2, 3, 4, 5, 6, 7, 8, 9, 10, 15, or 20 residues from, a naturally occurring SLAM family member.

In some embodiments, the vector is an in vitro transcribed vector, e.g., a vector that transcribes RNA of a nucleic acid molecule described herein. In some embodiments, the nucleic acid sequence in the vector further comprises a poly(A) tail, e.g., a poly A tail. In some embodiments, the nucleic acid sequence in the vector further comprises a 3′UTR, e.g., a 3′ UTR described herein, e.g., comprising at least one repeat of a 3′UTR derived from human beta-globulin. In some embodiments, the nucleic acid sequence in the vector further comprises promoter, e.g., a T2A promoter.

Promoters

In some embodiments, the vector further comprises a promoter. In some embodiments, the promoter is chosen from an EF-1 promoter, a CMV IE gene promoter, an EF-1a promoter, an ubiquitin C promoter, or a phosphoglycerate kinase (PGK) promoter. In some embodiments, the promoter is an EF-1 promoter. In some embodiments, the EF-1 promoter comprises a sequence of SEQ ID NO: 1.

In some aspects of the present invention, immune effector cells, e.g., T cells, can be obtained from a unit of blood collected from a subject using any number of techniques known to the skilled artisan, such as Ficoll™ separation. In some aspects, cells from the circulating blood of an individual are obtained by apheresis. The apheresis product typically contains lymphocytes, including T cells, monocytes, granulocytes, B cells, other nucleated white blood cells, red blood cells, and platelets. In some aspects, the cells collected by apheresis may be washed to remove the plasma fraction and, optionally, to suspend the cells in a buffer or medium for subsequent processing steps. In some embodiments, the cells are washed with phosphate buffered saline (PBS). In an alternative embodiment, the wash solution lacks calcium and may lack magnesium or may lack many if not all divalent cations.

TABLE 17
Sequences of various components of CAR (aa - amino acids, na - nucleic acids that
encodes the corresponding protein)

| SEQ | | |
|---|---|---|
| ID | | |
| NO | description | Sequence |
| 1 | EF-1 | CGTGAGGCTCCGGTGCCCGTCAGTGGGCAGAGCGCACATCGC |
| | promoter | CCACAGTCCCCGAGAAGTTGGGGGGAGGGGTCGGCAATTGA |
| | | ACCGGTGCCTAGAGAAGGTGGCGCGGGGTAAACTGGGAAAG |
| | | TGATGTCGTGTACTGGCTCCGCCTTTTTCCCGAGGGTGGGGG |
| | | AGAACCGTATATAAGTGCAGTAGTCGCCGTGAACGTTCTTTT |
| | | TCGCAACGGGTTTGCCGCCAGAACACAGGTAAGTGCCGTGTG |
| | | TGGTTCCCGCGGGCCTGGCCTCTTTACGGGTTATGGCCCTTGC |
| | | GTGCCTTGAATTACTTCCACCTGGCTGCAGTACGTGATTCTTG |
| | | ATCCCGAGCTTCGGGTTGGAAGTGGGTGGGAGAGTTCGAGGC |
| | | CTTGCGCTTAAGGAGCCCCTTCGCCTCGTGCTTGAGTTGAGGC |
| | | CTGGCCTGGGCGCTGGGGCCGCCGCGTGCGAATCTGGTGGCA |
| | | CCTTCGCGCCTGTCTCGCTGCTTTCGATAAGTCTCTAGCCATT |
| | | TAAAATTTTTGATGACCTGCTGCGACGCTTTTTTTCTGGCAAG |
| | | ATAGTCTTGTAAATGCGGGCCAAGATCTGCACACTGGTATTT |
| | | CGGTTTTTGGGGCCGCGGGCGGCGACGGGGCCCGTGCGTCCC |
| | | AGCGCACATGTTCGGCGAGGCGGGGCCTGCGAGCGCGGCCA |
| | | CCGAGAATCGGACGGGGGTAGTCTCAAGCTGGCCGGCCTGCT |
| | | CTGGTGCCTGGCCTCGCGCCGCCGTGTATCGCCCCGCCCTGG |
| | | GCGGCAAGGCTGGCCCGGTCGGCACCAGTTGCGTGAGCGGA |
| | | AAGATGGCCGCTTCCCGGCCCTGCTGCAGGGAGCTCAAAATG |
| | | GAGGACGCGGCGCTCGGGAGAGCGGGCGGGTGAGTCACCCA |
| | | CACAAAGGAAAAGGGCCTTTCCGTCCTCAGCCGTCGCTTCAT |
| | | GTGACTCCACGGAGTACCGGGCGCCGTCCAGGCACCTCGATT |
| | | AGTTCTCGAGCTTTTGGAGTACGTCGTCTTTAGGTTGGGGGG |
| | | AGGGGTTTTATGCGATGGAGTTTCCCCACACTGAGTGGGTGG |
| | | AGACTGAAGTTAGGCCAGCTTGGCACTTGATGTAATTCTCCTT |
| | | GGAATTTGCCCTTTTTGAGTTTGGATCTTGGTTCATTCTCAAG |
| | | CCTCAGACAGTGGTTCAAAGTTTTTTTCTTCCATTTCAGGTGT |
| | | CGTGA |
| 2 | Leader (aa) | MALPVTALLLPLALLLHAARP |
| 3 | Leader (na) | ATGGCCCTGCCTGTGACAGCCCTGCTGCTGCCTCTGGCTCTGC |
| | | TGCTGCATGCCGCTAGACCC |
| 4 | CD 8 hinge | TTTPAPRPPTPAPTIASQPLSLRPEACRPAAGGAVHTRGLDFACD |
| | (aa) | |
| 5 | CD8 hinge | ACCACGACGCCAGCGCCGCGACCACCAACACCGGCGCCCAC |
| | (na) | CATCGCGTCGCAGCCCCTGTCCCTGCGCCCAGAGGCGTGCCG |
| | | GCCAGCGGCGGGGGGCGCAGTGCACACGAGGGGGCTGGACT |
| | | TCGCCTGTGAT |
| 6 | Ig4 hinge | ESKYGPPCPPCPAPEFLGGPSVFLFPPKPKDTLMISRTPEVTCVVV |
| | (aa) | DVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTP |
| | | PVLDSDGSFFLYSRLTVDKSRWQEGNVFSCSVMHEALHNHYTQ |
| | | KSLSLSLGKM |
| 7 | Ig4 hinge | GAGAGCAAGTACGGCCCTCCCTGCCCCCCTTGCCCTGCCCCC |
| | (na) | GAGTTCCTGGGCGGACCCAGCGTGTTCCTGTTCCCCCCCAAG |
| | | CCCAAGGACACCCTGATGATCAGCCGGACCCCCGAGGTGACC |
| | | TGTGTGGTGGTGGACGTGTCCCAGGAGGACCCCGAGGTCCAG |
| | | TTCAACTGGTACGTGGACGGCGTGGAGGTGCACAACGCCAAG |
| | | ACCAAGCCCCGGGAGGAGCAGTTCAATAGCACCTACCGGGT |
| | | GGTGTCCGTGCTGACCGTGCTGCACCAGGACTGGCTGAACGG |
| | | CAAGGAATACAAGTGTAAGGTGTCCAACAAGGGCCTGCCCA |
| | | GCAGCATCGAGAAAACCATCAGCAAGGCCAAGGGCCAGCCT |
| | | CGGGAGCCCCAGGTGTACACCCTGCCCCCTAGCCAAGAGGAG |
| | | ATGACCAAGAACCAGGTGTCCCTGACCTGCCTGGTGAAGGGC |
| | | TTCTACCCCAGCGACATCGCCGTGGAGTGGGAGAGCAACGGC |
| | | CAGCCCGAGAACAACTACAAGACCACCCCCCCTGTGCTGGAC |
| | | AGCGACGGCAGCTTCTTCCTGTACAGCCGGCTGACCGTGGAC |
| | | AAGAGCCGGTGGCAGGAGGGCAACGTCTTTAGCTGCTCCGTG |
| | | ATGCACGAGGCCCTGCACAACCACTACACCCAGAAGAGCCTG |
| | | AGCCTGTCCCTGGGCAAGATG |
| 8 | IgD hinge | RWPESPKAQASSVPTAQPQAEGSLAKATTAPATTRNTGRGGEE |
| | (aa) | KKKEKEKEEQEERETKTPECPSHTQPLGVYLLTPAVQDLWLRD |
| | | KATFTCFVVGSDLKDAHLTWEVAGKVPTGGVEEGLLERHSNGS |
| | | QSQHSRLTLPRSLWNAGTSVTCTLNHPSLPPQRLMALREPAAQA |
| | | PVKLSLNLLASSDPPEAASWLLCEVSGFSPPNILLMWLEDQREV |
| | | NTSGFAPARPPPQPGSTTFWAWSVLRVPAPPSPQPATYTCVVSH |
| | | EDSRTLLNASRSLEVSYVTDH |
| 9 | IgD hinge | AGGTGGCCCGAAAGTCCCAAGGCCCAGGCATCTAGTGTTCCT |
| | (na) | ACTGCACAGCCCCAGGCAGAAGGCAGCCTAGCCAAAGCTAC |
| | | TACTGCACCTGCCACTACGCGCAATACTGGCCGTGGCGGGGA |
| | | GGAGAAGAAAAAGGAGAAAGAGAAAGAAGAACAGGAAGAG |
| | | AGGGAGACCAAGACCCCTGAATGTCCATCCCATACCCAGCCG |
| | | CTGGGCGTCTATCTCTTGACTCCCGCAGTACAGGACTTGTGGC |
| | | TTAGAGATAAGGCCACCTTTACATGTTTCGTCGTGGGCTCTGA |
| | | CCTGAAGGATGCCCATTTGACTTGGGAGGTTGCCGGAAAGGT |
| | | ACCCACAGGGGGGGTTGAGGAAGGGTTGCTGGAGCGCCATT |
| | | CCAATGGCTCTCAGAGCCAGCACTCAAGACTCACCCTTCCGA |
| | | GATCCCTGTGGAACGCCGGGACCTCTGTCACATGTACTCTAA |
| | | ATCATCCTAGCCTGCCCCCACAGCGTCTGATGGCCCTTAGAG |
| | | AGCCAGCCGCCCAGGCACCAGTTAAGCTTAGCCTGAATCTGC |
| | | TCGCCAGTAGTGATCCCCCAGAGGCCGCCAGCTGGCTCTTAT |
| | | GCGAAGTGTCCGGCTTTAGCCCGCCCAACATCTTGCTCATGT |
| | | GGCTGGAGGACCAGCGAGAAGTGAACACCAGCGGCTTCGCT |
| | | CCAGCCCGGCCCCCACCCCAGCCGGGTTCTACCACATTCTGG |
| | | GCCTGGAGTGTCTTAAGGGTCCCAGCACCACCTAGCCCCCAG |
| | | CCAGCCACATACACCTGTGTTGTGTCCCATGAAGATAGCAGG |
| | | ACCCTGCTAAATGCTTCTAGGAGTCTGGAGGTTTCCTACGTG |
| | | ACTGACCATT |
| 10 | GS | GGGGSGGGGS |
| | hinge/linker | |
| | (aa) | |
| 11 | GS | GGTGGCGGAGGTTCTGGAGGTGGAGGTTCC |
| | hinge/linker | |
| | (na) | |
| 12 | CD8 TM | IYIWAPLAGTCGVLLLSLVITLYC |
| | (aa) | |
| 13 | CD8 TM | ATCTACATCTGGGCGCCCTTGGCCGGGACTTGTGGGGTCCTTC |
| | (na) | TCCTGTCACTGGTTATCACCCTTTACTGC |
| 14 | 4-1BB | KRGRKKLLYIFKQPFMRPVQTTQEEDGCSCRFPEEEEGGCEL |
| | intracellular | |
| | domain | |
| | (aa) | |
| 15 | 4-1BB | AAACGGGGCAGAAAGAAACTCCTGTATATATTCAAACAACC |
| | intracellular | ATTTATGAGACCAGTACAAACTACTCAAGAGGAAGATGGCTG |
| | domain | TAGCTGCCGATTTCCAGAAGAAGAAGAAGGAGGATGTGAAC |
| | (na) | TG |
| 16 | CD27 (aa) | QRRKYRSNKGESPVEPAEPCRYSCPREEEGSTIPIQEDYRKPEPA |
| | | CSP |
| 17 | CD27 (na) | AGGAGTAAGAGGAGCAGGCTCCTGCACAGTGACTACATGAA |
| | | CATGACTCCCCGCCGCCCCGGGCCCACCCGCAAGCATTACCA |
| | | GCCCTATGCCCCACCACGCGACTTCGCAGCCTATCGCTCC |
| 18 | CD3-zeta | RVKFSRSADAPAYKQGQNQLYNELNLGRREEYDVLDKRRGRD |
| | (aa) | PEMGGKPRRKNPQEGLYNELQKDKMAEAYSEIGMKGERRRGK |
| | | GHDGLYQGLSTATKDTYDALHMQALPPR |
| 19 | CD3-zeta | AGAGTGAAGTTCAGCAGGAGCGCAGACGCCCCCGCGTACAA |
| | (na) | GCAGGGCCAGAACCAGCTCTATAACGAGCTCAATCTAGGACG |
| | | AAGAGAGGAGTACGATGTTTTGGACAAGAGACGTGGCCGGG |
| | | ACCCTGAGATGGGGGGAAAGCCGAGAAGGAAGAACCCTCAG |
| | | GAAGGCCTGTACAATGAACTGCAGAAAGATAAGATGGCGGA |
| | | GGCCTACAGTGAGATTGGGATGAAAGGCGAGCGCCGGAGGG |
| | | GCAAGGGGCACGATGGCCTTTACCAGGGTCTCAGTACAGCCA |
| | | CCAAGGACACCTACGACGCCCTTCACATGCAGGCCCTGCCCC |
| | | CTCGC |
| 20 | CD3-zeta | RVKFSRSADAPAYQQGQNQLYNELNLGRREEYDVLDKRRGRD |
| | (aa) | PEMGGKPRRKNPQEGLYNELQKDKMAEAYSEIGMKGERRRGK |
| | | GHDGLYQGLSTATKDTYDALHMQALPPR |
| 21 | CD3-zeta | AGAGTGAAGTTCAGCAGGAGCGCAGACGCCCCCGCGTACCA |
| | (na) | GCAGGGCCAG |
| | | AACCAGCTCTATAACGAGCTCAATCTAGGACGAAGAGAGGA |
| | | GTACGATGTTT |
| | | TGGACAAGAGACGTGGCCGGGACCCTGAGATGGGGGGAAAG |
| | | CCGAGAAGGA |
| | | AGAACCCTCAGGAAGGCCTGTACAATGAACTGCAGAAAGAT |
| | | AAGATGGCGG |
| | | AGGCCTACAGTGAGATTGGGATGAAAGGCGAGCGCCGGAGG |
| | | GGCAAGGGGC |
| | | ACGATGGCCTTTACCAGGGTCTCAGTACAGCCACCAAGGACA |
| | | CCTACGACGC |
| | | CCTTCACATGCAGGCCCTGCCCCCTCGC |
| 22 | linker | GGGGS |
| 23 | linker | GGTGGCGGAGGTTCTGGAGGTGGAGGTTCC |
| 24 | PD-1 | Pgwfldspdrpwnpptfspallvvtegdnatftcsfsntsesfylnwyrmspsnqtdklaafpedr |
| | extracellular | sqpgqdcrfrvtqlpngrdfhmsvvrarrndsgtylcgaislapkaqikeslraelrvterraevpta |
| | domain | hpspsprpagqfqtlv |
| | (aa) | |
| 25 | PD-1 | Cccggatggtttctggactctccggatcgcccgtggaatcccccaaccttctcaccggcactcttggtt |
| | extracellular | gtgactgagggcgataatgcgaccttcacgtgctcgttctccaacacctccgaatcattcgtgctgaact |
| | domain | ggtaccgcatgagcccgtcaaaccagaccgacaagctcgccgcgtttccggaagatcggtcgcaac |
| | (na) | cgggacaggattgtcggttccgcgtgactcaactgccgaatggcagagacttccacatgagcgtggt |
| | | ccgcgctaggcgaaacgactccgggacctacctgtgcggagccatctcgctggcgcctaaggccca |
| | | aatcaaagagagcttgagggccgaactgagagtgaccgagcgcagagctgaggtgccaactgcac |
| | | atccatccccatcgcctcggcctgcggggcagtttcagaccctggtc |
| 26 | PD-1 CAR | Malpvtalllplalllhaarppgwfldspdrpwnpptfspallvvtegdnatftcsfsntsesfylnw |
| | (aa) with | yrmspsnqtdklaafpedrsqpgqdcrfrvtqlpngrdfhmsvvrarrndsgtylcgaislapkaq |
| | signal | ikeslraelrvterraevptahpspsprpagqfqtlvtttpaprpptpaptiasqplslrpeacrpaagg |
| | | avhtrgldfacdiyiwaplagtcgvlllslvitlyckrgrkkllyifkqpfmrpvqttqeedgcscrfp |
| | | eeeeggcelrvkfsrsadapaykqgqnqlynelnlgrreeydvldkrrgrdpemggkprrknpqe |
| | | glynelqkdkmaeayseigmkgerrrgkghdglyqglstatkdtydalhmqalppr |
| 27 | PD-1 CAR | Atggccctccctgtcactgccctgcttctccccctcgcactcctgctccacgccgctagaccacccgg |
| | (na) | atggtttctggactctccggatcgcccgtggaatcccccaaccttctcaccggcactcttggttgtgact |
| | | gagggcgataatgcgaccttcacgtgctcgttctccaacacctccgaatcattcgtgctgaactggtac |
| | | cgcatgagcccgtcaaaccagaccgacaagctcgccgcgtttccggaagatcggtcgcaaccggg |
| | | acaggattgtcggttccgcgtgactcaactgccgaatggcagagacttccacatgagcgtggtccgc |
| | | gctaggcgaaacgactccgggacctacctgtgcggagccatctcgctggcgcctaaggcccaaatc |
| | | aaagagagcttgagggccgaactgagagtgaccgagcgcagagctgaggtgccaactgcacatcc |
| | | atccccatcgcctcggcctgcggggcagtttcagaccctggtcacgaccactccggcgccgcgccc |
| | | accgactccggccccaactatcgcgagccagcccctgtcgctgaggccggaagcatgccgccctgc |
| | | cgccggaggtgctgtgcatacccggggattggacttcgcatgcgacatctacatttgggctcctctcgc |
| | | cggaacttgtggcgtgctccttctgtccctggtcatcaccctgtactgcaagcggggtcggaaaaagct |
| | | tctgtacattttcaagcagccatcatgaggcccgtgcaaaccacccaggaggaggacggttgctcct |
| | | gccggttccccgaagaggaagaaggaggttgcgagctgcgcgtgaagttctcccggagcgccgac |
| | | gcccccgcctataagcagggccagaaccagctgtacaacgaactgaacctgggacggcgggaaga |
| | | gtacgatgtgctggacaagcggcgcggccgggaccccgaaatgggcgggaagcctagaagaaag |
| | | aaccctcaggaaggcctgtataacgagctgcagaaggacaagatggccgaggcctactccgaaatt |
| | | gggatgaagggagagcggcggaggggaaaggggcacgacggcctgtaccaaggactgtccacc |
| | | gccaccaaggacacatacgatgccctgcacatgcaggcccttccccctcgc |
| 28 | linker | (Gly-Gly-Gly-Ser)n, where n = 1-10 |
| 29 | linker | (Gly4 Ser)4 |
| 30 | linker | (Gly4 Ser)3 |
| 31 | linker | (Gly3Ser) |
| 39 | PD1 CAR | Pgwfldspdrpwnpptfspallvvtegdnatftcsfsntsesfvlnwyrmspsnqtdklaafpedr |
| | (aa) | sqpgqdcrfrvtqlpngrdfhmsvvrarrndsgtylcgaislapkaqikeslraelrvterraevpta |
| | | hpspsprpagqfqtlvtttpaprpptpaptiasqplslrpeacrpaaggavhtrgldfacdiyiwapla |
| | | gtcgvlllslvitlyckrgrkkllyifkqpfmrpvqttqeedgcscrfpeeeeggcelrvkfsrsadap |
| | | aykqgqnqlynelnlgrreeydvldkrrgrdpemggkprrknpqeglynelqkdkmaeaysei |
| | | gmkgerrrgkghdglyqglstatkdtydalhmqalppr |

In Vitro CAR-T Manufacture

While methods contemplated herein relate to in vivo transduction of cells, the challenges of in vitro manufacture are also appreciated.

In some embodiments, cells transduced the viral vector as described herein, are expanded, e.g., by a method described herein. In some embodiments, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In some embodiments, the cells are expanded for a period of 4 to 9 days. In some embodiments, the cells are expanded for a period of 8 days or less, e.g., 7, 6 or 5 days. In some embodiments, the cells are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In some embodiments, the cells are expanded for 5 days show at least a one, two, three or four fold increase in cells doublings upon antigen stimulation as compared to the same cells expanded in culture for 9 days under the same culture conditions. In some embodiments, the cells are expanded in culture for 5 days, and the resulting cells exhibit higher proinflammatory cytokine production, e.g., IFN-γ and/or GM-CSF levels, as compared to the same cells expanded in culture for 9 days under the same culture conditions. In some embodiments, the cells expanded for 5 days show at least a one, two, three, four, five, ten fold or more increase in μg/ml of proinflammatory cytokine production, e.g., IFN-γ and/or GM-CSF levels, as compared to the same cells expanded in culture for 9 days under the same culture conditions.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the in vitro methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In some aspects, T cells are isolated from peripheral blood lymphocytes by lysing the red blood cells and depleting the monocytes, for example, by centrifugation through a PERCOLL′ gradient or by counterflow centrifugal elutriation. The isolated T cells may be further used in the methods described herein.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In some embodiments, T regulatory cells, e.g., CD25+ T cells, are removed from the population using an anti-CD25 antibody, or fragment thereof, or a CD25-binding ligand, IL-2. In some embodiments, the anti-CD25 antibody, or fragment thereof, or CD25-binding ligand is conjugated to a substrate, e.g., a bead, or is otherwise coated on a substrate, e.g., a bead. In some embodiments, the anti-CD25 antibody, or fragment thereof, is conjugated to a substrate as described herein.

In some embodiments, the T regulatory cells, e.g., CD25+ T cells, are removed from the population using CD25 depletion reagent from Miltenyi™. In some embodiments, the ratio of cells to CD25 depletion reagent is 1×10⁷ cells to 20 μL, or 1×10⁷ cells to 15 μL, or 1×10⁷ cells to 10 μL, or 1×10⁷ cells to 5 μL, or 1×10⁷ cells to 2.5 μL, or 1×10⁷ cells to 1.25 μL. In some embodiments, e.g., for T regulatory cells, e.g., CD25+ depletion, greater than 500 million cells/ml is used. In a further aspect, a concentration of cells of 600, 700, 800, or 900 million cells/ml is used.

In some embodiments, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In some embodiments, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In some embodiments, the T regulatory cells, e.g., CD25+ cells, are removed from the population using the CliniMAC system with a depletion tubing set, such as, e.g., tubing 162-01. In some embodiments, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., TREG cells), in a subject prior to apheresis or during manufacturing of a CAR-expressing cell product can reduce the risk of subject relapse. For example, methods of depleting TREG cells are known in the art. Methods of decreasing TREG cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) TREG cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete TREG cells prior to manufacturing of the CAR-expressing cell (e.g., T cell, NK cell) product.

In some embodiments, a subject is pre-treated with one or more therapies that reduce TREG cells prior to collection of cells for CAR-expressing cell product manufacturing, thereby reducing the risk of subject relapse to CAR-expressing cell treatment. In some embodiments, methods of decreasing TREG cells include, but are not limited to, administration to the subject of one or more of cyclophosphamide, anti-GITR antibody, CD25-depletion, or a combination thereof.

Administration of one or more of cyclophosphamide, anti-GITR antibody, CD25-depletion, or a combination thereof, can occur before, during or after an infusion of the CAR-expressing cell product.

In some embodiments, a subject is pre-treated with cyclophosphamide prior to collection of cells for CAR-expressing cell product manufacturing, thereby reducing the risk of subject relapse to CAR-expressing cell treatment. In some embodiments, a subject is pre-treated with an anti-GITR antibody prior to collection of cells for CAR-expressing cell product manufacturing, thereby reducing the risk of subject relapse to CAR-expressing cell treatment.

In some embodiments, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In some embodiments, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In some embodiments, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B7-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In some embodiments, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can isolated by incubation with anti-CD3/anti-CD28 (e.g., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. In some embodiments, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In some aspects, it may be desirable to significantly decrease the volume in which beads and cells are mixed together (e.g., increase the concentration of cells), to ensure maximum contact of cells and beads. For example, in some aspects, a concentration of 10 billion cells/ml, 9 billion/ml, 8 billion/ml, 7 billion/ml, 6 billion/ml, or 5 billion/ml is used. In some aspects, a concentration of 1 billion cells/ml is used. In yet some aspects, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a some embodiments, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In some aspects, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, can be isolated and frozen for later use in immune effector cell therapy for any number of diseases or conditions that would benefit from immune effector cell therapy, such as those described herein. In some aspects a blood sample or an apheresis is taken from a generally healthy subject. In some aspects, a blood sample or an apheresis is taken from a generally healthy subject who is at risk of developing a disease, but who has not yet developed a disease, and the cells of interest are isolated and frozen for later use.

In some aspects, the T cells may be expanded, frozen, and used at a later time. In some aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In some embodiments, the NK cells are obtained from the subject. In another embodiment, the NK cells are an NK cell line, e.g., NK-92 cell line (Conkwest).

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods described herein. Subjects in need thereof may subsequently undergo standard treatment with high dose chemotherapy followed by peripheral blood stem cell transplantation. In some aspects, following or concurrent with the transplant, subjects receive an infusion of the expanded CART cells as prepared by the methods of the present invention. In an additional aspect, expanded cells are administered before or following surgery.

Additional Expressed Agents

In the embodiments contemplated herein, it is appreciated that additional agents may be encoded in the vectors described herein above. Accordingly, these agents are described below in relation to the CAR-expressing cell.

In another embodiment, a CAR-expressing immune effector cell described herein can further express another agent, e.g., an agent which enhances the activity of a CAR-expressing cell. For example, in some embodiments, the agent can be an agent which inhibits an inhibitory molecule. Examples of inhibitory molecules include PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGFR beta, e.g., as described herein. In some embodiments, the agent that inhibits an inhibitory molecule comprises a first polypeptide, e.g., an inhibitory molecule, associated with a second polypeptide that provides a positive signal to the cell, e.g., an intracellular signaling domain described herein. In some embodiments, the agent comprises a first polypeptide, e.g., of an inhibitory molecule such as PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 or TGFR beta, or a fragment of any of these, and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In some embodiments, the agent comprises a first polypeptide of PD-1 or a fragment thereof, and a second polypeptide of an intracellular signaling domain described herein (e.g., a CD28, CD27, OX40 or 4-IBB signaling domain described herein and/or a CD3 zeta signaling domain described herein).

In some embodiments, the CAR-expressing immune effector cell described herein can further comprise a second CAR, e.g., a second CAR that includes a different antigen binding domain, e.g., to the same target (e.g., a target described above) or a different target. In some embodiments, the second CAR includes an antigen binding domain to a target expressed on the same cancer cell type as the target of the first CAR. In some embodiments, the CAR-expressing immune effector cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-IBB, CD28, CD27 or OX-40, onto the first CAR, and the primary signaling domain, e.g., CD3 zeta, on the second CAR can limit the CAR activity to cells where both targets are expressed. In some embodiments, the CAR expressing immune effector cell comprises a first CAR that includes an antigen binding domain that targets, e.g., a target described above, a transmembrane domain and a costimulatory domain and a second CAR that targets an antigen other than antigen targeted by the first CAR (e.g., an antigen expressed on the same cancer cell type as the first target) and includes an antigen binding domain, a transmembrane domain and a primary signaling domain. In another embodiment, the CAR expressing immune effector cell comprises a first CAR that includes an antigen binding domain that targets, e.g., a target described above, a transmembrane domain and a primary signaling domain and a second CAR that targets an antigen other than antigen targeted by the first CAR (e.g., an antigen expressed on the same cancer cell type as the first target) and includes an antigen binding domain to the antigen, a transmembrane domain and a costimulatory signaling domain.

In some embodiments, the CAR-expressing immune effector cell comprises a CAR described herein, e.g., a CAR to a target described above, and an inhibitory CAR. In some embodiments, the inhibitory CAR comprises an antigen binding domain that binds an antigen found on normal cells but not cancer cells, e.g., normal cells that also express the target. In some embodiments, the inhibitory CAR comprises the antigen binding domain, a transmembrane domain and an intracellular domain of an inhibitory molecule. For example, the intracellular domain of the inhibitory CAR can be an intracellular domain of PD1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 or TGFR beta.

In some embodiments, an immune effector cell (e.g., T cell, NK cell) comprises a first CAR comprising an antigen binding domain that binds to a tumor antigen as described herein, and a second CAR comprising a PD1 extracellular domain or a fragment thereof.

In some embodiments, the cell further comprises an inhibitory molecule as described above.

In some embodiments, the second CAR in the cell is an inhibitory CAR, wherein the inhibitory CAR comprises an antigen binding domain, a transmembrane domain, and an intracellular domain of an inhibitory molecule. The inhibitory molecule can be chosen from one or more of: PD1, PD-L1, CTLA-4, TIM-3, LAG-3, VISTA, BTLA, TIGIT, LAIR1, CD160, 2B4, TGFR beta, CEACAM-1, CEACAM-3, and CEACAM-5. In some embodiments, the second CAR molecule comprises the extracellular domain of PD1 or a fragment thereof.

In embodiments, the second CAR molecule in the cell further comprises an intracellular signaling domain comprising a primary signaling domain and/or an intracellular signaling domain.

In other embodiments, the intracellular signaling domain in the cell comprises a primary signaling domain comprising the functional domain of CD3 zeta and a costimulatory signaling domain comprising the functional domain of 4-1BB.

In some embodiments, the antigen binding domain of the first CAR molecule comprises a scFv and the antigen binding domain of the second CAR molecule does not comprise a scFv. For example, the antigen binding domain of the first CAR molecule comprises a scFv and the antigen binding domain of the second CAR molecule comprises a camelid VHH domain.

Conformation of CARs

In the embodiments contemplated herein, it is appreciated that the conformation of one or more CARs could be modulated by the vectors described herein above. Accordingly, these conformations are described below in relation to the CAR-expressing cell.

Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

Multiple CAR

In some aspects, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein). In some embodiments, the second CAR includes an antigen binding domain to a target expressed the same cancer cell type as the cancer associated antigen. In some embodiments, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, CD27 or OX-40, onto the first CAR, and the primary signaling domain, e.g., CD3 zeta, on the second CAR can limit the CAR activity to cells where both targets are expressed. In some embodiments, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In some embodiments, the claimed invention comprises a first and second CAR, wherein the antigen binding domain of one of said first CAR said second CAR does not comprise a variable light domain and a variable heavy domain. In some embodiments, the antigen binding domain of one of said first CAR said second CAR is an scFv, and the other is not an scFv. In some embodiments, the antigen binding domain of one of said first CAR said second CAR comprises a single VH domain, e.g., a camelid, shark, or lamprey single VH domain, or a single VH domain derived from a human or mouse sequence. In some embodiments, the antigen binding domain of one of said first CAR said second CAR comprises a nanobody. In some embodiments, the antigen binding domain of one of said first CAR said second CAR comprises a camelid VHH domain.

Once the methods described herein are performed, various assays can be used to evaluate the activity of, for e.g., the ability to expand T cells following antigen stimulation, sustain T cell expansion in the absence of re-stimulation, and anti-cancer activities in appropriate in vitro and animal models. Assays to evaluate the effects of a CAR of the present invention are known to those of skill in the art and generally described below.

Western blot analysis of CAR expression in primary T cells can be used to detect the presence of monomers and dimers. See, e.g., Milone et al., Molecular Therapy 17(8): 1453-1464 (2009). Very briefly, T cells (1:1 mixture of CD4⁺ and CD8⁺ T cells) expressing the CARs are expanded in vitro for more than 10 days followed by lysis and SDS-PAGE under reducing conditions. CARs containing the full length TCR-ζ cytoplasmic domain and the endogenous TCR-ζ chain are detected by western blotting using an antibody to the TCR-ζ chain. The same T cell subsets are used for SDS-PAGE analysis under non-reducing conditions to permit evaluation of covalent dimer formation.

In vitro expansion of CAR⁺ T cells following antigen stimulation can be measured by flow cytometry.

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR on day 1.

Animal models can also be used to measure a CART activity. For example, xenograft model using human a cancer associated antigen described herein-specific CAR′ T cells to treat a primary human pre-B ALL in immunodeficient mice can be used. See, e.g., Milone et al., Molecular Therapy 17(8): 1453-1464 (2009).

Dose dependent CAR treatment response can be evaluated. See, e.g., Milone et al., Molecular Therapy 17(8): 1453-1464 (2009). For example, peripheral blood is obtained 35-70 days after establishing leukemia in mice injected on day 21 with CAR T cells, an equivalent number of mock-transduced T cells, or no T cells. Mice from each group are randomly bled for determination of peripheral blood a cancer associate antigen as described herein⁺ ALL blast counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009).

Cytotoxicity can be assessed by a standard 51Cr-release assay. See, e.g., Milone et al., Molecular Therapy 17(8): 1453-1464 (2009

Imaging technologies can be used to evaluate specific trafficking and proliferation of CARs in tumor-bearing animal models. Such assays have been described, for example, in Barrett et al., Human Gene Therapy 22:1575-1586 (2011).

Other assays, including those described in the Example section herein as well as those that are known in the art can also be used to evaluate the CARs described herein.

Methods of Treatment

In some embodiments, the invention is a method of treating a subject having a disease, disorder, or condition associated with an elevated expression of a tumor antigen, the method comprising: administering to a subject a composition comprising a first population of mesoporous silica particles and a viral vector;

wherein the viral vector comprises an expression vector comprising a recombinant polynucleotide comprising an expression control sequence operatively linked to a nucleotide sequence that expresses a chimeric antigen receptor (CAR) that is engineered to target the tumor antigen.

In yet another aspect, the invention features a method of treating a subject having a disease associated with expression of a tumor antigen (e.g., an antigen described herein), comprising administering to the subject an effective amount of a composition comprising mesoporous silica particles and a viral vector as described herein, wherein the viral vector comprises an expression vector comprising a recombinant polynucleotide comprising an expression control sequence operatively linked to a nucleotide sequence that expresses a chimeric antigen receptor (CAR) that is engineered to target the tumor antigen.

In some embodiments, the MSPs are rod shaped (MSRs). In some embodiments, the MSPs (e.g., MSRs) further comprise a plurality of functional groups adsorbed or covalently bonded onto the surfaces lining the pores and/or nanochannels or the surface of the MSPs or MSRs. As used herein, the “functional group” defines a chemical moiety linked to the surface of the MSR (e.g., MSP), either directly, or via a linker. In some embodiments, the functional group is a —OH (hydroxyl), amine, carboxylic acid, phosphonate, halide, azide, alkyne, epoxide, sulfhydryl, disulfide, polyethyleneimine, hydrophobic moiety or salts thereof. In some embodiments, the functional group (i.e. —OH (hydroxyl), amine, carboxylic acid, phosphonate, halide, azide, alkyne, epoxide, sulfhydryl, disulfide, polyethyleneimine, hydrophobic moiety, or salts thereof) may be separated from the silica surface by a linker. In some embodiments, the functional group is covalently bonded to the MSP surface via a C₁ to C₂₀ alkyl linker. In other embodiments, the functional group is covalently bonded to the MSP surface via a polyethyleneglycol linker. In particular embodiments, the polyethylene glycol linker has the formula (—O(CH₂—CH₂—)₁₋₂₅. In particular embodiments, the surface modification is a C₁ to C₂₀ alkyl perhaloalkyl or a C₁ to C₂₀ alkyl perfluoroalkyl.

In some embodiments of the described method, the electrostatic conjugation between the mesoporous silica particles and the viral vector is due to oppositely charged viral vectors and mesoporous silica particles. For example and without being bound by theory, mesoporous silica particles (e.g., MSRs) that are surface modified by polyethylene imine or primary, secondary, tertiary, or quarternary ammonium groups that are positively charged can be conjugated to or associated with negatively charged viral vectors. Thus in some embodiments, the viral vector is negatively charged and the mesoporous silica particles (e.g., MSRs) are positively charged. In some embodiments, the covalent conjugation between the mesoporous silica particles (e.g., MSRs) and the viral vector is achieved by methods known to those of skill in the art, either via linkers or without linkers. For example and without limitation, the linkers may be polyethylene glycol, alkyl groups, polymers, polyamide linkages, etc.

In some embodiments of the method, the composition further comprises a T cell stimulating compound or tumor antigen conjugated to or adsorbed on the first population of mesoporous silica particles or a second population of mesoporous silica particles, or both populations of MSPs (e.g., MSRs). Alternatively, the method includes administering a second population of mesoporous silica particles in combination with, e.g., simultaneously or shortly after, administration of the first population of MSPs (e.g., MSRs). Alternatively, the second population of MSPs (e.g., MSRs) may be administered after a prolonged period of time after administration of the first population of MSPs.

In some embodiments, the method comprises administering a cytokine, wherein the cytokine is conjugated to or adsorbed on the first or second population of mesoporous silica particles.

In some embodiments, the second population of MSPs (e.g., MSRs) is administered to the subject simultaneously (e.g., administered on the same day) with or shortly after administration (e.g., administered 1 day, 2 days, 3 days, 4 days, 5 days, 6 days, or 7 days after administration) of the first population of MSPs. In other embodiments, the cytokine is administered to the subject after a prolonged period of time (e.g., e.g., at least 2 weeks, 3 weeks, 4 weeks, 6 weeks, 8 weeks, 10 weeks, or more) after administration of the first population of MSPs.

In some aspects, in the methods recited herein, the mesoporous silica particles can be surface modified as described herein. In some embodiments, the MSPs (e.g., MSRs) further comprise a plurality of functional groups adsorbed or covalently bonded onto the surfaces lining the pores and/or nanochannels or the surface of the MSPs or MSRs. As used herein, the “functional group” defines a chemical moiety linked to the surface of the MSR or MSP, either directly, or via a linker. In some embodiments, the functional group is a —OH (hydroxyl), amine, carboxylic acid, phosphonate, halide, azide, alkyne, epoxide, sulfhydryl, disulfide, polyethyleneimine, hydrophobic moiety or salts thereof. In some embodiments, the functional group (i.e. —OH (hydroxyl), amine, carboxylic acid, phosphonate, halide, azide, alkyne, epoxide, sulfhydryl, disulfide, polyethyleneimine, hydrophobic moiety, or salts thereof) may be separated from the silica surface by a linker. In some embodiments, the functional group is covalently bonded to the MSP (e.g., MSR) surface via a C₁ to C₂₀ alkyl linker. In other embodiments, the functional group is covalently bonded to the MSP (e.g., MSR) surface via a polyethyleneglycol linker. In particular embodiments, the polyethylene glycol linker has the formula (—O(CH2-CH₂—)₁₋₂₅. In particular embodiments, the surface modification is a C₁ to C₂₀ alkyl perhaloalkyl or a C₁ to C₂₀ alkyl perfluoroalkyl.

In another aspect, in the methods described herein, the viral vector can be conjugated to the mesoporous silica particles (e.g., MSRs) as described herein. In some embodiments, the electrostatic conjugation between the mesoporous silica particles and the viral vector is due to oppositely charged viral vectors and mesoporous silica particles. For example and without being bound by theory, mesoporous silica particles (e.g., MSRs) that are surface modified by polyethylene imine or primary, secondary, tertiary, or quarternary ammonium groups that are positively charged can be conjugated to or associated with negatively charged viral vectors. Thus in some embodiments, the viral vector is negatively charged and the mesoporous silica particles are positively charged. In some embodiments, the covalent conjugation between the mesoporous silica particles and the viral vector is achieved by methods known to those of skill in the art, either via linkers or without linkers. For example and without limitation, the linkers may be polyethylene glycol, alkyl groups, polymers, polyamide linkages, etc.

In particular embodiments, the viral vector includes a comprises an expression vector comprising a recombinant polynucleotide comprising an expression control sequence operatively linked to a nucleotide sequence that expresses a chimeric antigen receptor (CAR) that is engineered to target the tumor antigen. Exemplary CARS are described herein.

In some embodiments of any of the aforesaid methods or uses, the disease associated with a tumor antigen, e.g., a tumor antigen described herein, is selected from a proliferative disease such as a cancer or malignancy or a precancerous condition such as a myelodysplasia, a myelodysplastic syndrome or a preleukemia, or is a non-cancer related indication associated with expression of a tumor antigen described herein. In some embodiments, the disease is a cancer described herein, e.g., a cancer described herein as being associated with a target described herein. In some embodiments, the disease is a hematologic cancer. In some embodiments, the hematologic cancer is leukemia. In some embodiments, the cancer is selected from the group consisting of one or more acute leukemias including but not limited to B-cell acute lymphoid leukemia (“BALL”), T-cell acute lymphoid leukemia (“TALL”), acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and to disease associated with expression of a tumor antigen described herein include, but not limited to, atypical and/or non-classical cancers, malignancies, precancerous conditions or proliferative diseases expressing a tumor antigen as described herein; and any combination thereof. In another embodiment, the disease associated with a tumor antigen described herein is a solid tumor.

In embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin [symbol] Disease, non-Hodgkin [symbol] lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi sarcoma, epidermoid cancer, squamous cell cancer, T-cell lymphoma, environmentally induced cancers, combinations of said cancers, and metastatic lesions of said cancers.

In some embodiments, a cancer that can be treated with CAR-expressing cell of the present invention is multiple myeloma. Generally, myeloma cells are thought to be negative for a cancer associate antigen as described herein expression by flow cytometry. Thus, in some embodiments, a CD19 CAR, e.g., as described herein, may be used to target myeloma cells. In some embodiments, cars of the present invention therapy can be used in combination with one or more additional therapies, e.g., lenalidomide treatment.

In various aspects, the immune effector cells (e.g., T cells, NK cells) generated by the methods described herein and administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell or NK cell to the patient.

The invention also includes a type of cellular therapy where immune effector cells (e.g., T cells, NK cells) are modified, e.g., by in vitro or in vivo transcribed RNA, to transiently express a chimeric antigen receptor (CAR). The resultant cells are able to kill tumor cells in the subject or patient. Thus, in various aspects, the immune effector cells (e.g., T cells, NK cells) are present for less than one month, e.g., three weeks, two weeks, one week, after administration of the compositions as described herein.

Without wishing to be bound by any particular theory, the anti-tumor immunity response elicited by the CAR-modified immune effector cells (e.g., T cells, NK cells) may be an active or a passive immune response, or alternatively may be due to a direct vs indirect immune response. In some aspects, the CAR transduced immune effector cells (e.g., T cells, NK cells) exhibit specific proinflammatory cytokine secretion and potent cytolytic activity in response to human cancer cells expressing the a cancer associate antigen as described herein, resist soluble a cancer associate antigen as described herein inhibition, mediate bystander killing and mediate regression of an established human tumor. For example, antigen-less tumor cells within a heterogeneous field of a cancer associate antigen as described herein-expressing tumor may be susceptible to indirect destruction by a cancer associate antigen as described herein-redirected immune effector cells (e.g., T cells, NK cells) that has previously reacted against adjacent antigen-positive cancer cells.

In some aspects, the fully-human CAR-modified immune effector cells (e.g., T cells, NK cells) of the invention may be a type of vaccine for ex vivo immunization and/or in vivo therapy in a mammal. In some aspects, the mammal is a human.

In some aspects the CAR-expressing cells of the inventions may be used to treat a proliferative disease such as a cancer or malignancy or is a precancerous condition such as a myelodysplasia, a myelodysplastic syndrome or a preleukemia. Further a disease associated with a cancer associate antigen as described herein expression include, but not limited to, e.g., atypical and/or non-classical cancers, malignancies, precancerous conditions or proliferative diseases expressing a cancer associated antigen as described herein. Non-cancer related indications associated with expression of a cancer associate antigen as described herein include, but are not limited to, e.g., autoimmune disease, (e.g., lupus), inflammatory disorders (allergy and asthma) and transplantation.

The CAR-modified immune effector cells (e.g., T cells, NK cells) of the present invention may be administered either alone, or as a pharmaceutical composition in combination with diluents and/or with other components such as IL-2 or other cytokines or cell populations.

Hematologic Cancer

Hematological cancer conditions are the types of cancer such as leukemia, lymphoma, and malignant lymphoproliferative conditions that affect blood, bone marrow and the lymphatic system.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

The present invention also provides methods for inhibiting the proliferation or reducing a cancer associated antigen as described herein, the methods comprising contacting a population of cells comprising a cancer associated antigen as described herein with a composition comprising a mesoporous silica particles and a viral vector. In a specific aspect, the MSPs are surface modified as described herein. In other embodiments, the viral vector comprises an expression vector comprising a recombinant polynucleotide comprising an expression control sequence operatively linked to a nucleotide sequence to be expressed. Exemplary nucleotide sequences express a chimeric antigen receptor (CAR), engineered TCR, cytokines, chemokines, shRNA to block an inhibitory molecule, or mRNA to induce expression of a protein. In some aspects, a CAR-expressing T cell or NK cell of the invention reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for myeloid leukemia or another cancer associated with a cancer associated antigen as described herein-expressing cells relative to a negative control. In some aspects, the subject is a human.

Combination Therapies

Administered “in combination”, as used herein, means that two (or more) different treatments are delivered to the subject during the course of the subject affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the methods or uses are carried out in combination with an agent that increases the efficacy of the immune effector cell, e.g., an agent as described herein.

In some embodiments of the methods or uses described herein, the mesoporous silica rod composition is administered in combination with an agent that increases the efficacy of the immune effector cell, e.g., one or more of a protein phosphatase inhibitor, a kinase inhibitor, a cytokine, an inhibitor of an immune inhibitory molecule; or an agent that decreases the level or activity of a TREG cell.

In some embodiments of the methods or uses described herein, the protein phosphatase inhibitor is a SHP-1 inhibitor and/or an SHP-2 inhibitor.

In other embodiments of the methods or uses described herein, kinase inhibitor is chosen from one or more of a CDK4 inhibitor, a CDK4/6 inhibitor (e.g., palbociclib), a BTK inhibitor (e.g., ibrutinib or RN-486), an mTOR inhibitor (e.g., rapamycin or everolimus (RAD001)), an MNK inhibitor, or a dual PI3K/mTOR inhibitor. In some embodiments, the BTK inhibitor does not reduce or inhibit the kinase activity of interleukin-2-inducible kinase (ITK).

In other embodiments of the methods or uses described herein, the agent that inhibits the immune inhibitory molecule comprises an antibody or antibody fragment, an inhibitory nucleic acid, a clustered regularly interspaced short palindromic repeats (CRISPR), a transcription-activator like effector nuclease (TALEN), or a zinc finger endonuclease (ZFN) that inhibits the expression of the inhibitory molecule.

In other embodiments of the methods or uses described herein, the agent that decreases the level or activity of the TREG cells is chosen from cyclophosphamide, anti-GITR antibody, CD25-depletion, or a combination thereof.

In some embodiments of the methods or uses described herein, the immune inhibitory molecule is selected from the group consisting of PD1, PD-L1, CTLA-4, TIM-3, LAG-3, VISTA, BTLA, TIGIT, LAIR1, CD160, 2B4, TGFR beta, CEACAM-1, CEACAM-3, and CEACAM-5.

In other embodiments, the agent that inhibits the inhibitory molecule comprises a first polypeptide comprising an inhibitory molecule or a fragment thereof and a second polypeptide that provides a positive signal to the cell, and wherein the first and second polypeptides are expressed on the CAR-containing immune cells, wherein (i) the first polypeptide comprises PD1, PD-L1, CTLA-4, TIM-3, LAG3, VISTA, BTLA, TIGIT, LAIR1, CD160, 2B4, TGFR beta, CEACAM-1, CEACAM-3, and CEACAM-5 or a fragment thereof; and/or (ii) the second polypeptide comprises an intracellular signaling domain comprising a primary signaling domain and/or a costimulatory signaling domain. In some embodiments, the primary signaling domain comprises a functional domain of CD3 zeta; and/or the costimulatory signaling domain comprises a functional domain of a protein selected from 41BB, CD27 and CD28.

In other embodiments, cytokine is chosen from IL-7, IL-15 or IL-21, or combinations thereof.

In other embodiments, the immune effector cell comprising the CAR molecule and a second, e.g., any of the combination therapies disclosed herein (e.g., the agent that that increases the efficacy of the immune effector cell) are administered substantially simultaneously or sequentially.

In other embodiments, the immune cell comprising the CAR molecule is administered in combination with a molecule that targets GITR and/or modulates GITR function. In some embodiments, the molecule targeting GITR and/or modulating GITR function is administered prior to the CAR-expressing cell or population of cells, or prior to apheresis.

In some embodiments, lymphocyte infusion, for example allogeneic lymphocyte infusion, is used in the treatment of the cancer, wherein the lymphocyte infusion comprises at least one CAR-expressing cell of the present invention. In some embodiments, autologous lymphocyte infusion is used in the treatment of the cancer, wherein the autologous lymphocyte infusion comprises at least one CAR-expressing cell described herein.

In some embodiments, the cell is a T cell and the T cell is diaglycerol kinase (DGK) deficient. In some embodiments, the cell is a T cell and the T cell is Ikaros deficient. In some embodiments, the cell is a T cell and the T cell is both DGK and Ikaros deficient.

In embodiments of any of the aforeseaid methods or uses, there may be a further administration of an agent that treats the disease associated with expression of the tumor antigen, e.g., any of the second or third therapies disclosed herein. Additional exemplary combinations include one or more of the following.

In another embodiment, there may be a further administration of another agent, e.g., a kinase inhibitor and/or checkpoint inhibitor described herein. For example, there may be a further administration of an agent which enhances the activity of a CAR-expressing cell.

For example, in some embodiments, the agent that enhances the activity of a CAR-expressing cell can be an agent which inhibits an inhibitory molecule (e.g., an immune inhibitor molecule). Examples of inhibitory molecules include PD1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGFR beta.

In some embodiments, the agent that inhibits the inhibitory molecule is an inhibitory nucleic acid is a dsRNA, a siRNA, or a shRNA. In embodiments, the inhibitory nucleic acid is linked to the nucleic acid that encodes a component of the CAR molecule. For example, the inhibitory molecule can be expressed on the CAR-expressing cell.

In another embodiment, the agent which inhibits an inhibitory molecule, e.g., is a molecule described herein, e.g., an agent that comprises a first polypeptide, e.g., an inhibitory molecule, associated with a second polypeptide that provides a positive signal to the cell, e.g., an intracellular signaling domain described herein. In some embodiments, the agent comprises a first polypeptide, e.g., of an inhibitory molecule such as PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 or TGFR beta, or a fragment of any of these (e.g., at least a portion of the extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In some embodiments, the agent comprises a first polypeptide of PD1 or a fragment thereof (e.g., at least a portion of the extracellular domain of PD1), and a second polypeptide of an intracellular signaling domain described herein (e.g., a CD28 signaling domain described herein and/or a CD3 zeta signaling domain described herein).

In some embodiments, the CAR-expressing immune effector cell of the present invention, e.g., T cell or NK cell, is administered to a subject that has received a previous stem cell transplantation, e.g., autologous stem cell transplantation.

In some embodiments, the CAR-expressing immune effector cell of the present invention, e.g., T cell or NK cells, is administered to a subject that has received a previous dose of melphalan.

In some embodiments, the cell expressing a CAR molecule, e.g., a CAR molecule described herein, is administered in combination with an agent that increases the efficacy of a cell expressing a CAR molecule, e.g., an agent described herein.

In some embodiments, the cell expressing a CAR molecule, e.g., a CAR molecule described herein, is administered in combination with an agent that ameliorates one or more side effect associated with administration of a cell expressing a CAR molecule, e.g., an agent described herein.

In some embodiments, the cell expressing a CAR molecule, e.g., a CAR molecule described herein, is administered in combination with an agent that treats the disease associated with a cancer associated antigen as described herein, e.g., an agent described herein.

In some embodiments, a cell expressing two or more CAR molecules, e.g., as described herein, is administered to a subject in need thereof to treat cancer. In some embodiments, a population of cells including a CAR expressing cell, e.g., as described herein, is administered to a subject in need thereof to treat cancer.

In some embodiments of the methods or uses described herein, the CAR molecule is administered in combination with another agent. In some embodiments, the agent can be a kinase inhibitor, e.g., a CDK4/6 inhibitor, a BTK inhibitor, an mTOR inhibitor, a MNK inhibitor, or a dual PI3K/mTOR inhibitor, and combinations thereof. In some embodiments, the kinase inhibitor is a CDK4 inhibitor, e.g., a CDK4 inhibitor described herein, e.g., a CD4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In some embodiments, the kinase inhibitor is a BTK inhibitor, e.g., a BTK inhibitor described herein, such as, e.g., ibrutinib. In some embodiments, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In some embodiments, the kinase inhibitor is a MNK inhibitor, e.g., a MNK inhibitor described herein, such as, e.g., 4-amino-5-(4-fluoroanilino)-pyrazolo[3,4-d] pyrimidine. The MNK inhibitor can be, e.g., a MNK1a, MNK1b, MNK2a and/or MNK2b inhibitor. The dual PI3K/mTOR inhibitor can be, e.g., PF-04695102.

In some embodiments of the methods or uses described herein, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N-[5-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 5-[3-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N-[4-(methylsulfonyl)phenyl]-2-pyrimidinamine (AZD5438); and XL281 (BMS908662).

In some embodiments of the methods or uses described herein, the kinase inhibitor is a CDK4 inhibitor, e.g., palbociclib (PD0332991), and the palbociclib is administered at a dose of about 50 mg, 60 mg, 70 mg, 75 mg, 80 mg, 90 mg, 100 mg, 105 mg, 110 mg, 115 mg, 120 mg, 125 mg, 130 mg, 135 mg (e.g., 75 mg, 100 mg or 125 mg) daily for a period of time, e.g., daily for 14-21 days of a 28 day cycle, or daily for 7-12 days of a 21 day cycle. In some embodiments, 1, 2, 3, 4, 5, 6, 7, 8, 9, 10, 11, 12 or more cycles of palbociclib are administered.

In some embodiments of the methods or uses described herein, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In some embodiments, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In some embodiments of the methods or uses described herein, the kinase inhibitor is a BTK inhibitor, e.g., ibrutinib (PCI-32765), and the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In some embodiments, 1, 2, 3, 4, 5, 6, 7, 8, 9, 10, 11, 12 or more cycles of ibrutinib are administered.

In some embodiments of the methods or uses described herein, the kinase inhibitor is a BTK inhibitor that does not inhibit the kinase activity of ITK, e.g., RN-486, and RN-486 is administered at a dose of about 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg (e.g., 150 mg, 200 mg or 250 mg) daily for a period of time, e.g., daily a 28 day cycle. In some embodiments, 1, 2, 3, 4, 5, 6, 7, or more cycles of RN-486 are administered.

In some embodiments of the methods or uses described herein, the kinase inhibitor is an mTOR inhibitor selected from temsirolimus; ridaforolimus (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N²-[1,4-dioxo-4-[[4-(4-oxo-8-phenyl-4H-1-benzopyran-2-yl)morpholinium-4-yl]methoxy]butyl]-L-arginylglycyl-L-α-aspartyl L-serine− (SEQ ID NO: 692), inner salt (SF1126); and XL765.

In some embodiments of the methods or uses described herein, the kinase inhibitor is an mTOR inhibitor, e.g., rapamycin, and the rapamycin is administered at a dose of about 3 mg, 4 mg, 5 mg, 6 mg, 7 mg, 8 mg, 9 mg, 10 mg (e.g., 6 mg) daily for a period of time, e.g., daily for 21 day cycle, or daily for 28 day cycle. In some embodiments, 1, 2, 3, 4, 5, 6, 7, 8, 9, 10, 11, 12 or more cycles of rapamycin are administered. In some embodiments, the kinase inhibitor is an mTOR inhibitor, e.g., everolimus and the everolimus is administered at a dose of about 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg (e.g., 10 mg) daily for a period of time, e.g., daily for 28 day cycle. In some embodiments, 1, 2, 3, 4, 5, 6, 7, 8, 9, 10, 11, 12 or more cycles of everolimus are administered.

In some embodiments of the methods or uses described herein, the kinase inhibitor is an MNK inhibitor selected from CGP052088; 4-amino-3-(p-fluorophenylamino)-pyrazolo[3,4-d] pyrimidine (CGP57380); cercosporamide; ETC-1780445-2; and 4-amino-5-(4-fluoroanilino)-pyrazolo[3,4-d] pyrimidine.

In some embodiments of the methods or uses described herein, the kinase inhibitor is a dual phosphatidylinositol 3-kinase (PI3K) and mTOR inhibitor selected from 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF-04691502); N-[4-[[4-(Dimethylamino)-1-piperidinyl]carbonyl]phenyl]-N-[4-(4,6-di-4-morpholinyl-1,3,5-triazin-2-yl)phenyl]urea (PF-05212384, PKI-587); 2-Methyl-2-{4-[3-methyl-2-oxo-8-(quinolin-3-yl)-2,3-dihydro-1H-imidazo[4,5-c] quinolin-1-yl]phenyl}propanenitrile (BEZ-235); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo[4,5-c]quinolin-2(3H)-one Maleic acid (NVP-BGT226); 3-[4-(4-Morpholinylpyrido[3,2:4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); and N-[2-[(3,5-Dimethoxyphenyl)amino]quinoxalin-3-yl]-4-[(4-methyl-3-methoxyphenyl)carbonyl]aminophenylsulfonamide (XL765).

In some embodiments of the methods or uses described herein, there may be a further administration of a protein tyrosine phosphatase inhibitor, e.g., a protein tyrosine phosphatase inhibitor described herein. In some embodiments, the protein tyrosine phosphatase inhibitor is an SHP-1 inhibitor, e.g., an SHP-1 inhibitor described herein, such as, e.g., sodium stibogluconate. In some embodiments, the protein tyrosine phosphatase inhibitor is an SHP-2 inhibitor.

In some embodiments of the methods or uses described herein, there may be a further administration of another agent, and the agent is a cytokine. The cytokine can be, e.g., IL-7, IL-15, IL-21, or a combination thereof. In another embodiment, the CAR molecule is administered in combination with a checkpoint inhibitor, e.g., a checkpoint inhibitor described herein. For example, in some embodiments, the check point inhibitor inhibits an inhibitory molecule selected from PD-1, PD-L1, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGFR beta.

In other embodiments of the methods or uses described herein, there may be a further administration of an agent that ameliorates one or more side effects associated with a cell expressing a CAR molecule. Side effects associated with the CAR-expressing cell can be chosen from cytokine release syndrome (CRS) or hemophagocytic lymphohistiocytosis (HLH).

The present invention also provides methods for preventing, treating and/or managing a disease associated with a cancer associated antigen as described herein-expressing cells (e.g., a hematologic cancer or atypical cancer expressing a cancer associated antigen as described herein), the method comprising administering to a subject a composition comprising a first population of mesoporous silica particles and a viral vector, and wherein the viral vector comprises an expression vector comprising a recombinant polynucleotide comprising an expression control sequence operatively linked to a nucleotide sequence that expresses a chimeric antigen receptor (CAR) that is engineered to target the tumor antigen. In some aspects, the subject is a human. Non-limiting examples of disorders associated with a cancer associated antigen as described herein-expressing cells include autoimmune disorders (such as lupus), inflammatory disorders (such as allergies and asthma) and cancers (such as hematological cancers or atypical cancers expressing a cancer associated antigen as described herein).

The present invention also provides methods for preventing, treating and/or managing a disease associated with a cancer associated antigen as described herein-expressing cells, the methods comprising administering to a subject a composition comprising a first population of mesoporous silica particles and a viral vector, and wherein the viral vector comprises an expression vector comprising a recombinant polynucleotide comprising an expression control sequence operatively linked to a nucleotide sequence that expresses a chimeric antigen receptor (CAR) that is engineered to target the tumor antigen. In some aspects, the subject is a human.

The present invention provides methods for preventing relapse of cancer associated with a cancer associated antigen as described herein-expressing cells, the methods comprising administering to a subject a composition comprising a first population of mesoporous silica particles and a viral vector, and wherein the viral vector comprises an expression vector comprising a recombinant polynucleotide comprising an expression control sequence operatively linked to a nucleotide sequence that expresses a chimeric antigen receptor (CAR) that is engineered to target the tumor antigen.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject).

In some aspects, it may be desired to administer activated immune effector cells (e.g., T cells, NK cells) to a subject and then subsequently redraw blood (or have an apheresis performed), activate and expand immune effector cells (e.g., T cells, NK cells) according to the present invention, and reinfuse the patient with these activated and expanded immune effector cells (e.g., T cells, NK cells). This process can be carried out multiple times every few weeks. In some aspects, immune effector cells (e.g., T cells, NK cells) can be activated from blood draws of from 10cc to 400cc. In some aspects, immune effector cells (e.g., T cells, NK cells) are activated from blood draws of 20cc, 30cc, 40cc, 50cc, 60cc, 70cc, 80cc, 90cc, or 100cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In some aspects, the MSP (e.g., MSR) compositions of the present invention are administered to a patient by intradermal or subcutaneous injection. In some aspects, the T cell compositions of the present invention are administered parenterally. The term “parenteral” administration of an T cell composition includes, e.g., intrathecal, epidural, intracranial, subcutaneous (s.c.), intravenous (i.v.), intramuscular (i.m.), or intrasternal injection, intratumoral, or infusion techniques. In particular embodiments, the T cell composition is administered intravenously. In some embodiments, the compositions of MSPs (e.g., MSRs) and viral vectors may be injected directly into a tumor, lymph node, or site of infection.

### EXAMPLES

### Example A. Synthesis and Post-Functionalization of Mesoporous Silica Particles

Unless otherwise noted, all reagents were obtained from commercial sources and used as is.

### 1. Exemplary Synthesis of Mesoporous Silica Particles

Poly(ethylene glycol)-block-polypropylene glycol)-block-poly(ethylene glycol) avg Mn ˜5,800 (Pluronic P-123, 80.0 g, 487 mmol; Sigma) surfactant was dissolved in 3 L of 1.6M HCl at room temperature, heated to 40 degrees Celsius in a 5 L jacketed flask, and was mechanically stirred via and overhead stirrer at a rate of 0-600 rpm (but most commonly 300 rpm). Tetraethyl orthosilicate (TEOS, 184 mL, 826 mmol; Sigma) was added in one portion over <5 min and was heated at 40 degrees Celsius with maintained stirring for at least 2 hours but most commonly 20 hours. The resulting slurry was heated to 80-130 degrees Celsius (most commonly 100 degrees Celsius) for 6-72 hours (but most commonly 24 hours) for hydrothermal treatment before being cooled to room temperature. The slurry was filtered in a Buchner funnel and was washed with deionized water followed by ethanol and air dried at room temperature. The resulting silica material was calcined in a furnace with a slow ramp temperature from room temperature to 550 degrees Celsius over 8 hours and then maintaining at 550 degrees Celsius for another 8 hours before cooling to room temperature to afford 47g of mesoporous silica particles.

Changes in the stir rate may have changes in the microparticle aspect ratio. Varying the conditions of the hydrothermal temperature and duration are common pore size controllers for mesoporous materials. For more information, see J. Chem. Educ. 2017, 94, 91-94 and references within.

Final mesoporous materials were characterized by light microscopy, Malvern Morphologi G3, scanning electron microscopy (SEM), thermal gravimetric analysis (TGA).

### 2. Post-Modification of Silica Microparticles

### Example 2(a): Diethyl Ethylphosphonate Functionalized Microparticles

Diethyl ethylphosphonate functionalized silica microparticles were prepared by a modified method reported in New J. Chem., 2014, 38, 3853, with some modifications. Diethylphosphatoethyltriethoxysilane (4.15 mL, 13.03 mmol) was added to a slurry of 2.0 g of mesoporous silica microparticles suspended in 300 mL of toluene. The slurry was stirred and refluxed at 110 degrees Celsius for 14 hours before cooling to room temperature and filtered. The particles were washed with deionized water followed by ethanol and then dried in an oven at 100 degrees Celsius for 20 hours to afford diethyl ethylphosphonate functionalized particles.

### Example 2(b): Ethylphosphonic Acid Functionalized Microparticles

Ethylphosphonic acid functionalized microparticles were prepared by a modified method to the procedure reported in New J. Chem., 2014, 38, 3853. Trimethylsilylchlorosilane (1.388 mL, 10.86 mmol) was added to a slurry of 2.0 g of diethyl ethylphosphonate functionalized microparticles suspended in 150 mL of toluene and heated to 110 degrees Celsius for 24 hours. The slurry was cooled to room temperature and filtered, washing with dionized water and ethanol before drying in a oven at 100 degrees Celsius for 24 hours. The mesoporous silica particles were then suspended in 100 mL of 12M HCl and heated to 100 degrees Celsius for 18 hours. The slurry was cooled to room temperature, filtered and washed with deionized water and ethanol before drying in an oven at 100 degrees Celsius for 24 hours to afford ethylphosphonic acid functionalized microparticles.

### Example 2(c): Propylamine Functionalized Microparticles

Propylamine functionalized microparticles were prepared by a modified method to the procedure reported in Langmuir 2015, 31, 6457-6462. (3-aminopropyl)trimethoxysilane (3.05 ml, 19.54 mmol; APTMS, Sigma) was added to a slurry of 3.0 grams of mesoporous silica microparticles in 150 mL of reagent grade ethanol. The slurry was refluxed at 75 degrees Celsius for 7 hours. After cooling to room temperature and the slurry was filtered and the particles were washed with deionized water followed by ethanol and then dried in an oven at 100 degrees Celsius for 24 hours.

### Example 2(d): Biotin Functionalized Microparticles

(+)-Biotin N-succinimidyl ester (246 mg, 0.720 mmol) was added to a slurry of 1.0 g of propylamine-functionalized microparticles in 10.0 mL of pH 7.4 adjusted PBS buffer and stirred at room temperature for 18 hours. The slurry was filtered and washed with deionized water and ethanol before drying in an oven at 100 degrees Celsius for 24 hours to afford Biotin-functionalized microparticles.

### Example 2(e): Biotin-PEG4 Functionalized Microparticles

PEG4-Biotin N-hydroxysuccinimide (106 mg, 0.180 mmol; ThermoFischer EZ-Link NHS-PEG4-biotin) was added to a slurry of 0.25 g of propylamine-functionalized microparticles in 2.5 mL of pH 7.4 adjusted PBS buffer and stirred at room temperature for 18 hours. The slurry was filtered and washed with deionized water and ethanol before drying in an oven at 100 degrees Celsius for 24 hours to afford Biotin-PEG4 functionalized microparticles.

### Example 2(f): 3(2-pyridyldithio)propionamido)hexanoate Functionalized Microparticles

Succinimidyl 6-(3(2-pyridyldithio)propionamido)hexanoate (112 mg, 0.360 mmol; LC-SPDP, ThermoFischer) was added to a slurry of 0.50 g of propylamine-functionalized microparticles in 2.5 mL of pH 7.4 adjusted PBS buffer and stirred at room temperature for 18 hours. The slurry was filtered and washed with deionized water and ethanol before drying in an oven at 100 degrees Celsius for 24 hours to afford 3(2-pyridyldithio)propionamido)hexanoate-functionalized microparticles.

### Example 2(g): 4-oxo-4-(propylamino)butanoic Acid Functionalized Microparticles

Succinic anhydride (4 g, 40.0 mmol) was added to a slurry of 1.0 g of propylamine-functionalized microparticles in anhydrous DMF and was stirred at room temperature for 24 hours. The slurry was filtered and washed with deionized water and ethanol before drying in an oven at 100 degrees Celsius for 24 hours to afford 4-oxo-4-(propylamino)butanoic acid functionalized microparticles.

### Example 2(h): Propyldiethylenetriamine Functionalized Microparticles

Trimethoxysilylpropyldiethylenetriamine (1.678 mL, 6.51 mmol) was added to 1.0 g of mesoporous silica microparticles were suspended in 150 mL of reagent grade ethanol. The slurry was stirred at 75 degrees Celsius for 7 hours. After cooling to room temperature and the slurry was filtered and the particles were washed with deionized water followed by ethanol and then dried in an oven at 100 degrees Celsius for 20 hours to afford propyldiethylenetriamine functionalized microparticles.

### Example 2(i): 3-propyldihydrofuran-2,5-dione Functionalized Microparticles (Succinic Anhydride)

3-(3-(triethoxysilyl)propyl)dihydrofuran-2,5-dione (4.94 mL, 17.37 mmol) was added to a slurry of 3.0 g of mesoporous silica microparticles in 300 mL of toluene. The slurry was heated to 110 degrees Celsius for 20 hours and was then cooled to room temperature, filtered and washed with dionizied water and ethanol. The functionalized microparticles were dried in an oven at 100 degrees Celsius for 24 hours.

### Example 2(j): Branched, Polyethylenimine Functionalized Microparticles

Polyethylenimine (25.1 g, 47.0 mmol; Branched, avg Mw 25,000, Sigma) was dissolved in 600 mL of anhydrous DMF and 6.0 g of 3-propyldihydrofuran-2,5-dione functionalized microparticles were added and stirred at room temperature for 20 hours. The slurry was filtered and the particles were washed with deionized water followed by ethanol and then dried in an oven at 100 degrees Celsius for 20 hours to afford branched, polyethylenimine functionalized microparticles.

### Example 2(k): N,N,N-trimethylpropan-1-ammonium Functionalized Microparticles

Trimethoxysilylpropyltrimethylammonium chloride (3.61 mL, 6.51 mmol; 50% solution in methanol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford N,N,N-trimethylpropan-1-ammonium functionalized microparticles.

The procedure above was repeated at varying ratios of trimethoxysilylpropyltrimethylammonium chloride to silica microparticles (0.25 mmol trimethoxysilyltrimethylammonium chloride per gram of microparticles) to affect varying ratios of functional density.

### Example 2(l): Octyl Functionalized Microparticles

Triethyoxy(octyl)silane (2.05 mL, 6.51 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford octyl functionalized microparticles.

### Example 2(m): Hexadecyl Functionalized Microparticles

Hexadecyltrimethoxysilane (2.54 mL, 6.51 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford hexadecyl functionalized microparticles.

### Example 2(n): 11-azidoundecyl Functionalized Microparticles

(11-azidoundecyl)trimethoxysilane (1.0 g, 3.15 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford 11-azidoundecyl functionalized microparticles.

### Example 2(o): 3-azidopropyl Functionalized Microparticles

(3-azidopropyl)trimethoxysilane (1.0 g, 4.87 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford 3-azidopropyl functionalized microparticles.

### Example 2(p): 3,3,4,4,5,5,6,6,7,7,8,8,8-tridecafluorooctyl Functionalized Microparticles

Triethoxy(3,3,4,4,5,5,6,6,7,7,8,8,8-tridecafluorooctyl)silane (2.499 mL, 6.51 mmol) was added to a slurry of 1.0 g mesoporous silica microparticles in 150 mL reagent ethanol and was heated to 75 degrees Celsius for 7 hours. After cooling to room temperature and the slurry was filtered and the particles were washed with deionized water followed by ethanol and then dried in an oven at 100 degrees Celsius for 20 hours to afford 3,3,4,4,5,5,6,6,7,7,8,8,8-tridecafluorooctyl functionalized microparticles.

### Example B. Testing MSR Surface Modifications for Virus Binding

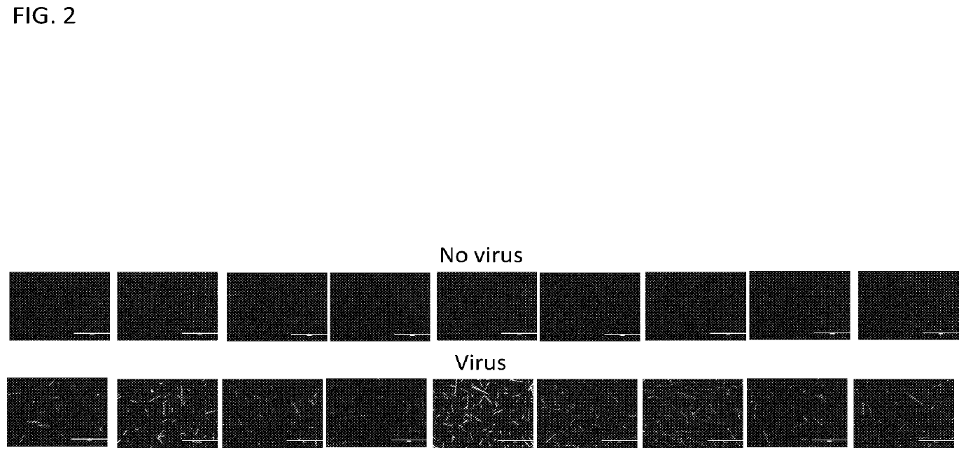

To test the binding of lentivirus to MSPs, a variety of MSPs was prepared with varying surface chemistries (FIG. 1). Dry MSR batches were resuspended at 10 mg/ml in ice-cold Tris-NaCl-EDTA buffer pH 7.5 (NTE buffer). A stock solution of green fluorescent protein (GFP) expressing lentivirus (FCT067, Kerafast) was diluted in ice-cold NTE buffer to a titer of 3×10⁶/ml. The MSR suspension and the diluted virus were combined at ratios of 1:1 vol/vol and incubated on ice for 30 minutes. Control particles were incubated 1:1 vol/vol with NTE buffer without virus. Following the incubation, samples were washed once with 1% bovine serum albumin (BSA) in phosphate buffered saline (PBS) at 4° C., and then with PBS at 4° C. Samples were then fixed with 4.2% paraformaldehyde in PBS. Samples were stained with an antibody against the viral envelope (anti-VSV-G from Kerafast, 8G5F11; 1:50 dilution) followed by an anti-mouse IgG labeled with Dylight-488 (Invitrogen). Samples were washed twice with PBS and imaged using an Evos fluorescent microscope equipped with a GFP LED light cube (FIG. 2). Imaging showed no detectable binding of the staining reagents to MSRs bearing no virus. Virus-conjugated rods show varying levels of quantitative binding, with the trimethylammonium and amine functionalities showing maximum binding.

### Example C. In Vitro Assay for T Cell Transduction with GFP Lentivirus Using MSR

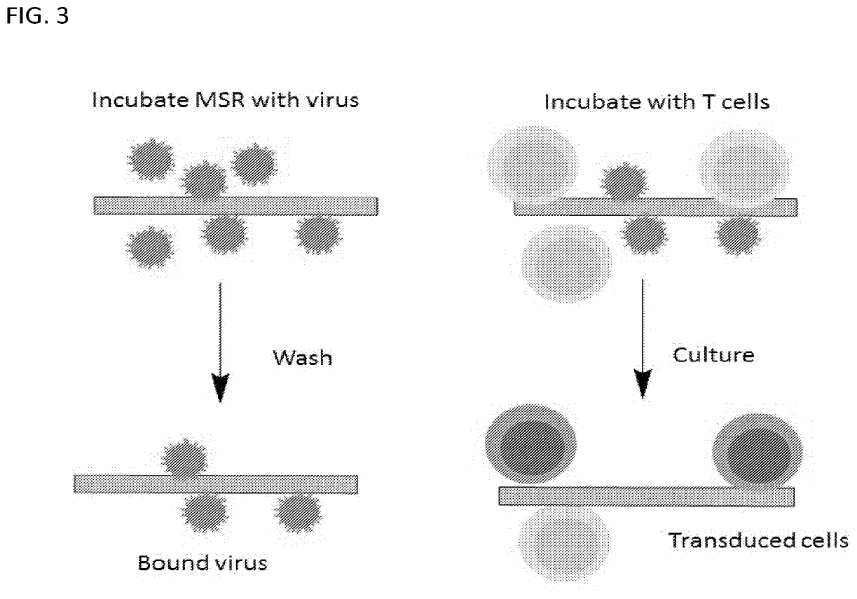

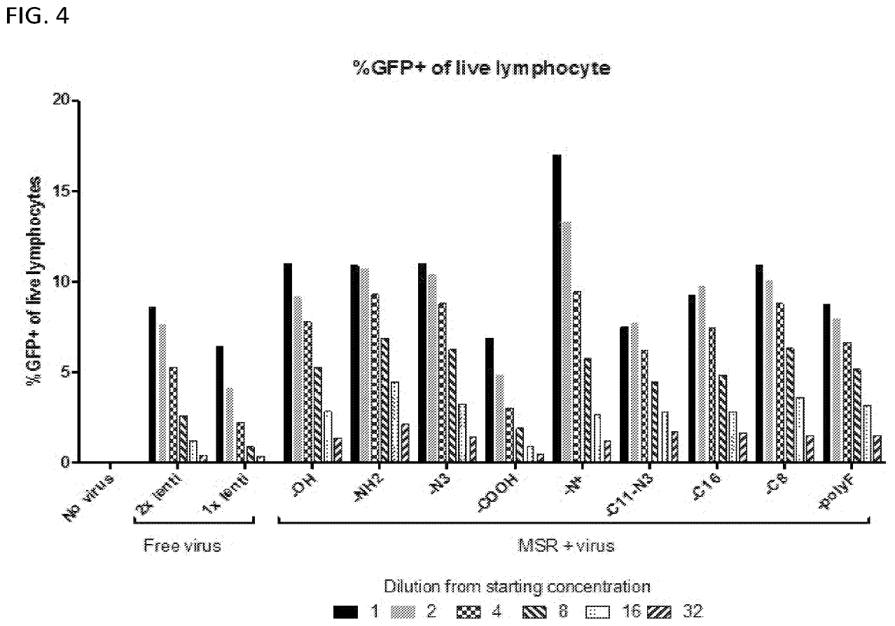

A schematic representation of the use of MSRs for virus transduction of T cells is shown in FIG. 3. Naïve human T cells were stimulated with Dynabead T cell activator beads at a 3:1 bead:cell ratio for two days. Beads were removed using a magnet, and cells were transferred to fresh culture medium. Virus-conjugated MSRs were prepared as noted above and resuspended at 80 μg/ml in cell culture medium. Serial dilutions of this were made as indicated in FIG. 3. This suspension was combined 1:1 with T cells 5×10⁵/ml and incubated for four days. GFP expression was assessed in live, singlet, cells in culture to assess the transduction efficiency. Results (FIG. 4) indicate that the transduction of MSR-conjugated virus occurred at greater levels than virus given in culture media only. The trimethylammonium functionalized MSRs provided the highest level of transduction.

### Example D. Interaction of T Cells with MSRs Presenting CD3/CD28 Agonistic Antibodies, EGFRvIII Peptides, or BCMA Protein

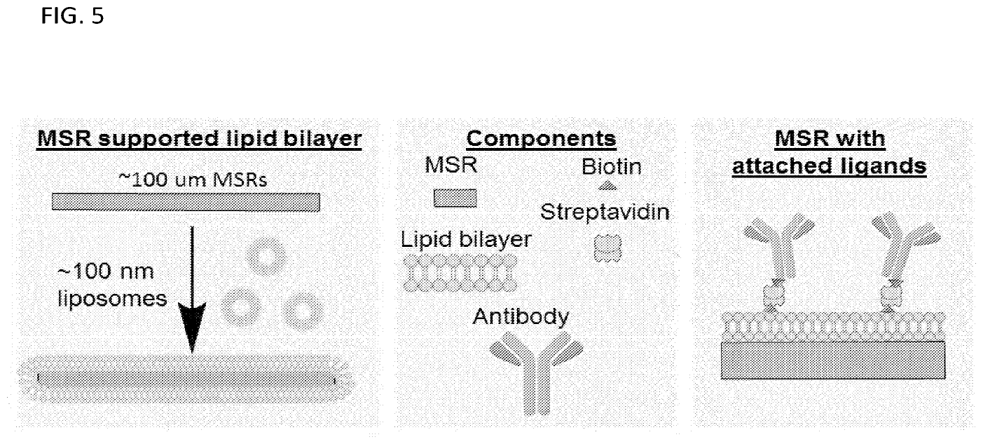

MSRs with surface-immobilized ligands were prepared as described in Cheung, A. S., et al., Scaffolds that mimic antigen-presenting cells enable ex vivo expansion of primary T cells. Nature Biotechnology, 36(2), 160-169. A schematic of this process is shown in FIG. 5.

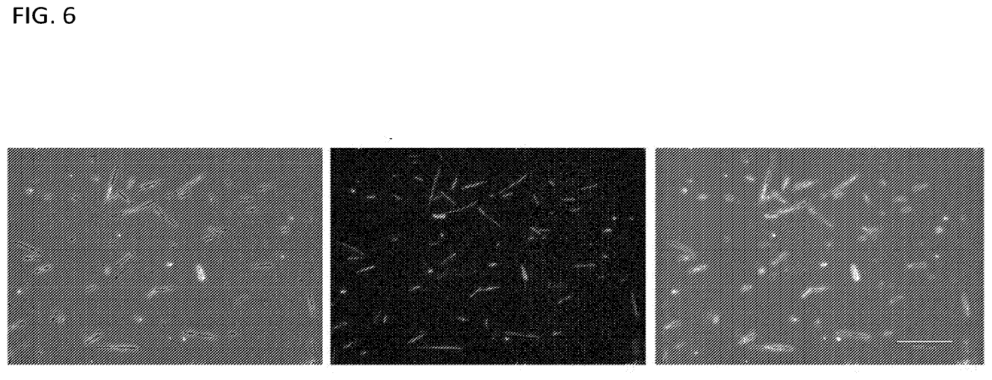

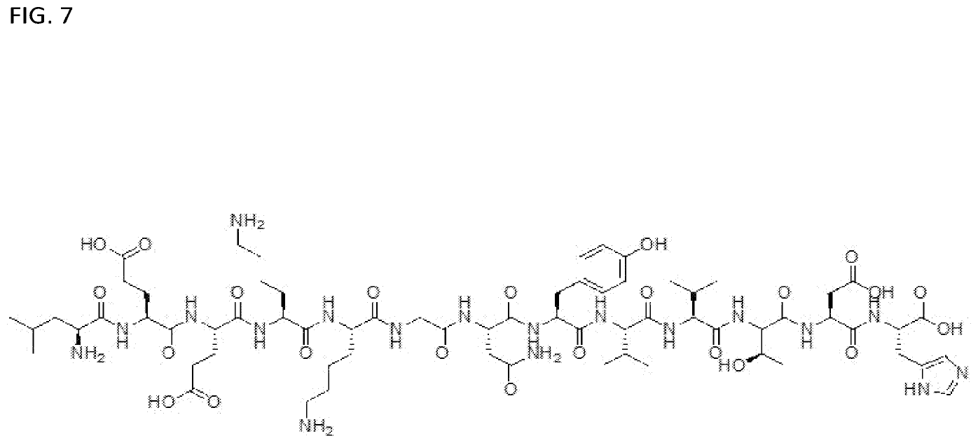

Briefly, liposomes primarily composed of POPC with 1 mol % PE-biotin were formed using a thin film rehydration method and extrusion through a 100 nm polycarbonate membrane. Hydroxyl functionalized MSRs were incubated with the liposomes to allow the formation of a supported lipid bilayer on the MSR surface (FIG. 6). To functionalize the MSRs with CD3 and CD28 agonistic antibodies, MSRs were washed several times with PBS, incubated with streptavidin, and then tethered with biotinylated CD3 and CD28 antibodies. For MSR-immobilization of EGFRvIII CAR-binding peptides, a biotinylated EGFRvIII CAR-binding peptide was used (FIG. 7). For BCMA CART stimulation, recombinant BCMAFc protein was biotinylated using biotin-NHS and similarly coupled to the MSR surface.

After incubation with the desired ligands, MSRs were washed several times with PBS and resuspended in culture medium at various concentrations and incubated with T cells. T cell proliferation was read out using CFSE labeling of the T cells and assessing dye dilution by flow cytometry. Cytokine production was assessed using a multiplex cytokine analysis method (Mesoscale Delivery V-Plex).

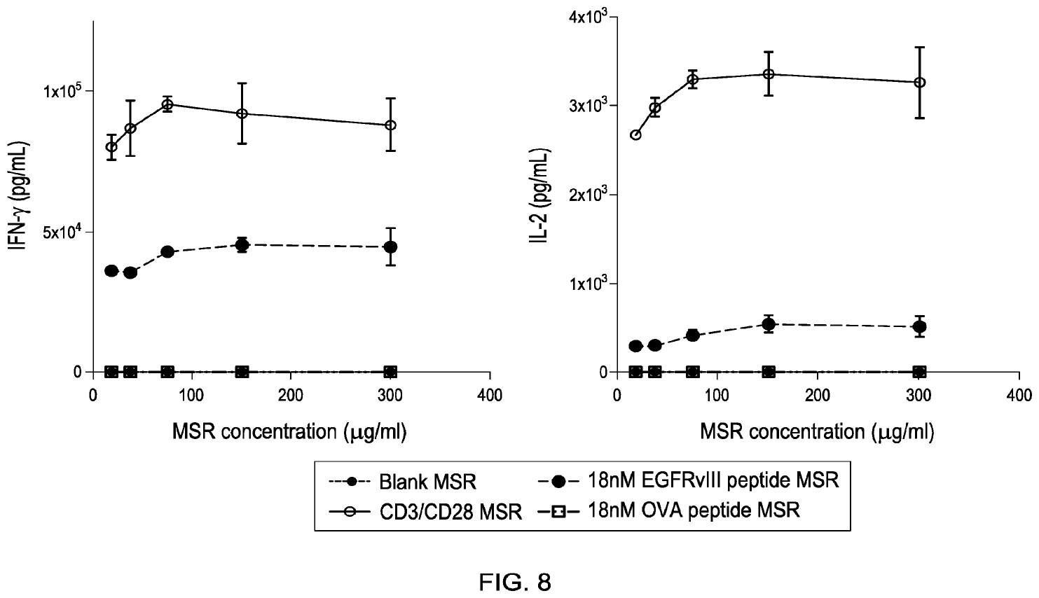

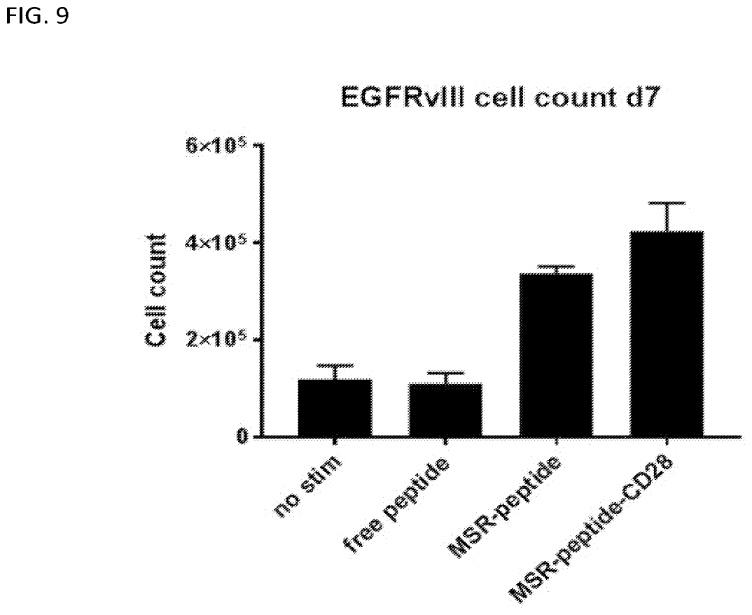

EGFRvIII CARTs produced interferon gamma and IL-2 in response to EGFRvIII CAR-binding peptide bound to the surface of the MSRs, while free EGFRvIII CAR-binding peptide in solution, a non-stimulating peptide (OVA) presented on the MSRs, or undecorated MSRs gave no response from the CARTs (FIG. 8). In another experiment, the proliferation of EGFRvIII CARTs were monitored in response to various stimuli using cell counting (FIG. 9).

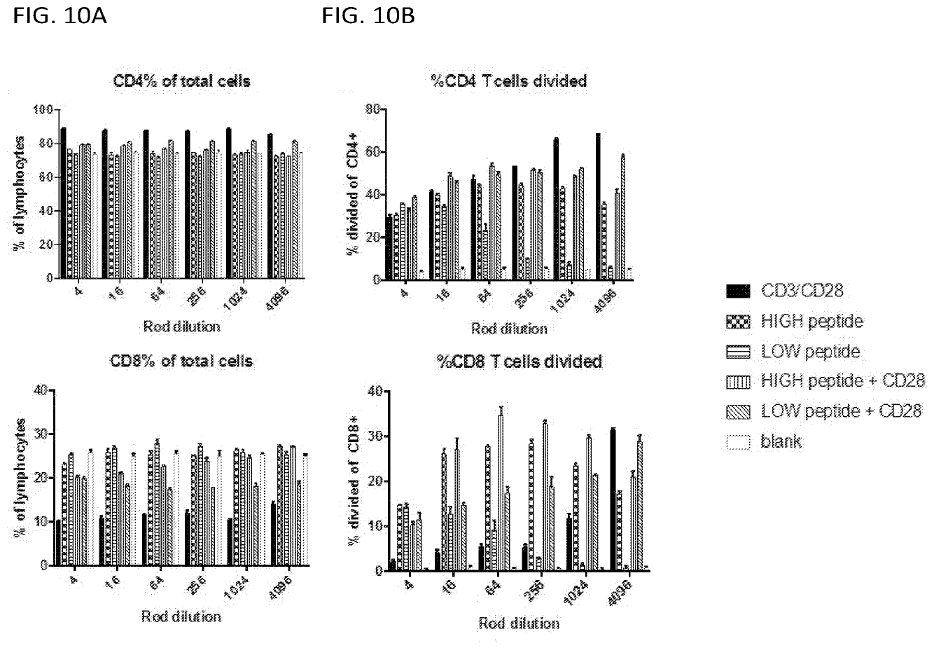

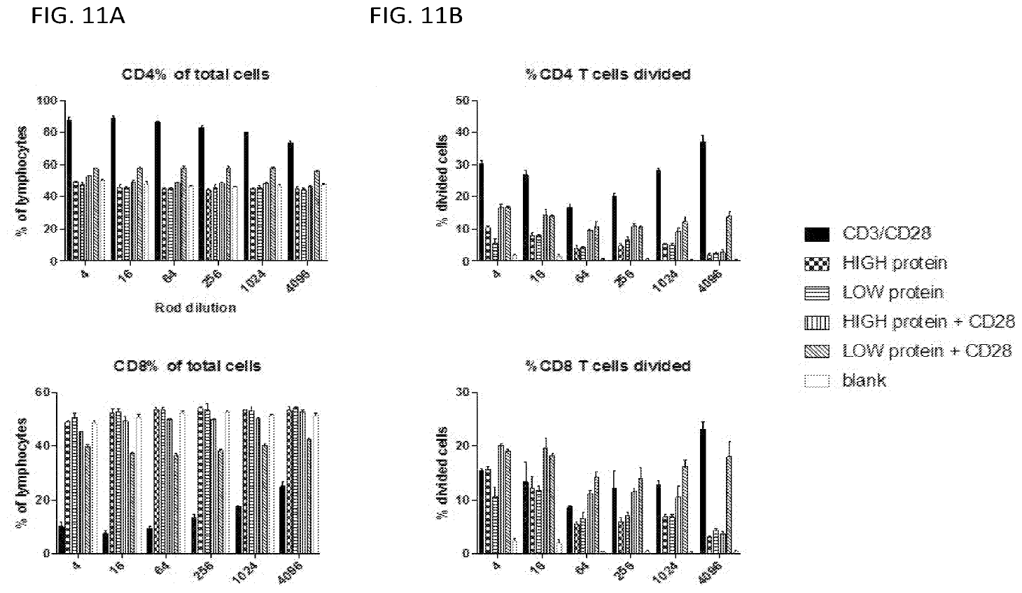

To further analyze the phenotype expansion of different T cell subsets, proliferation of EGFRvIII CARTs was assessed using flow cytometry. CARTs were stained with CFSE and monitored for dye dilution to indicate proliferation by flow cytometry (FIG. 10). Similar experiments were conducted using MSRs functionalized with BCMAFc protein antigen present on the MSR surface (FIG. 11).

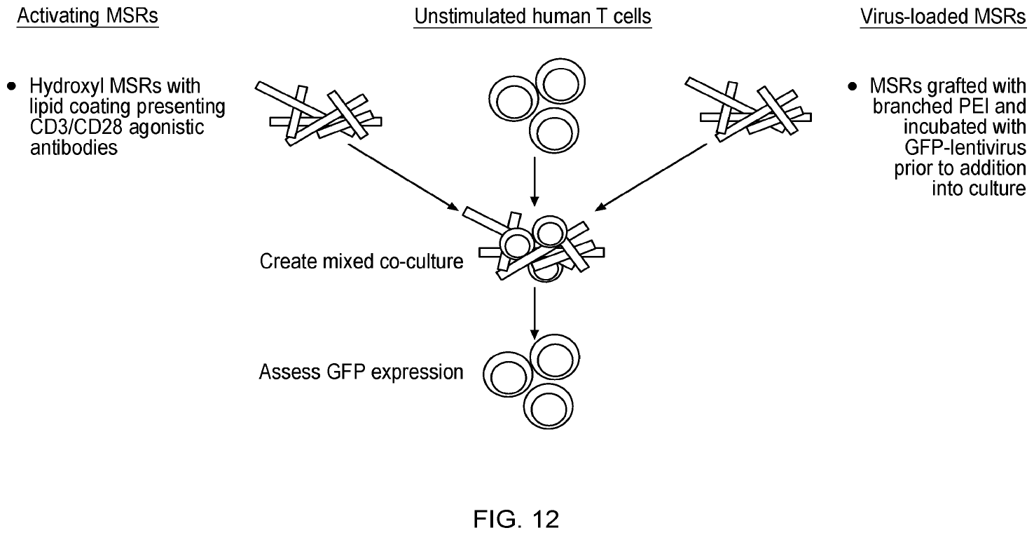

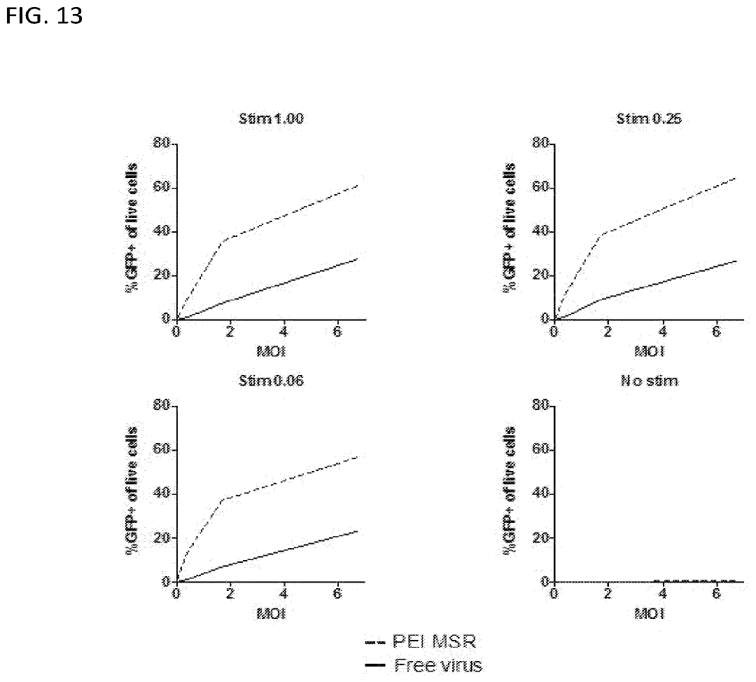

To test the simultaneous stimulation and transduction of T cells with virus using two types of MSRs (MSRs bearing stimulatory cues, and MSRs mixed with lentivirus), the experimental schema shown in FIG. 12 was used. One population of MSRs were coated with a lipid bilayer and grafted with anti-CD3/CD28 antibodies as described above. A second population of MSRs were incubated with lentivirus. Results shown in FIG. 13 indicated a superior transduction level when T cells were stimulated with anti-CD3/CD28 agonistic antibodies and were exposed to virus that was incubated with PEI-MSRs compared to free virus in solution.

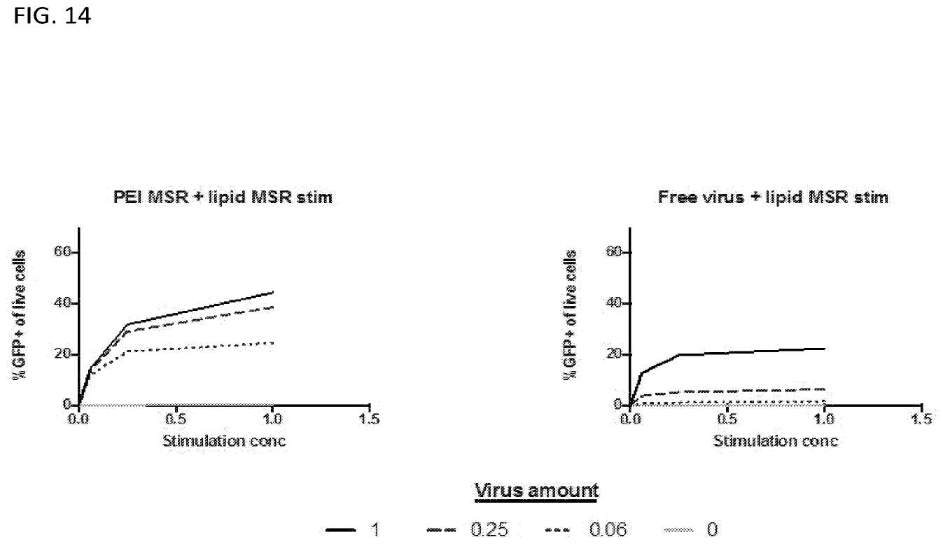

To test the simultaneous stimulation and transduction of T cells with both cues on the same population of MSRs, T cells were exposed to either (1) anti-CD3/CD28 agonistic antibody-bearing lipid-coated stimulating MSRs, and virus in media, (2) anti-CD3/CD28 agonistic antibody-bearing lipid-coated stimulating MSRs, and PEI-MSRs pre-incubated with virus, or (3) PEI MSRS adsorbed with anti-CD3/CD28 agonistic antibodies, and then incubated with virus. After three days of culture, T cells were assessed for transduction efficiency. FIG. 14 shows the effect of stimulatory MSR concentration on the MSRs of conditions (1) and (2) above at various amounts of virus. As shown in the FIG. 14, overall transduction is enhanced under condition (2) where PEI-MSRs are incubated with virus.

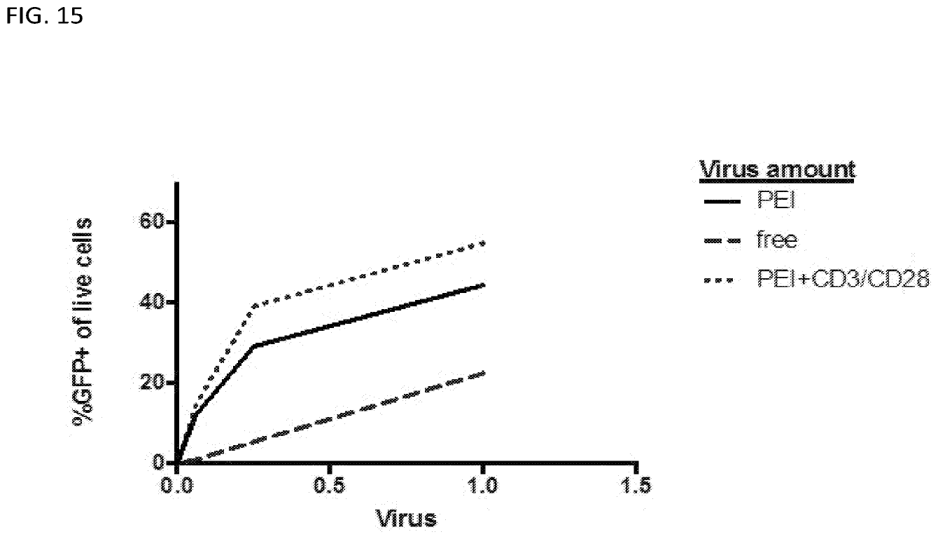

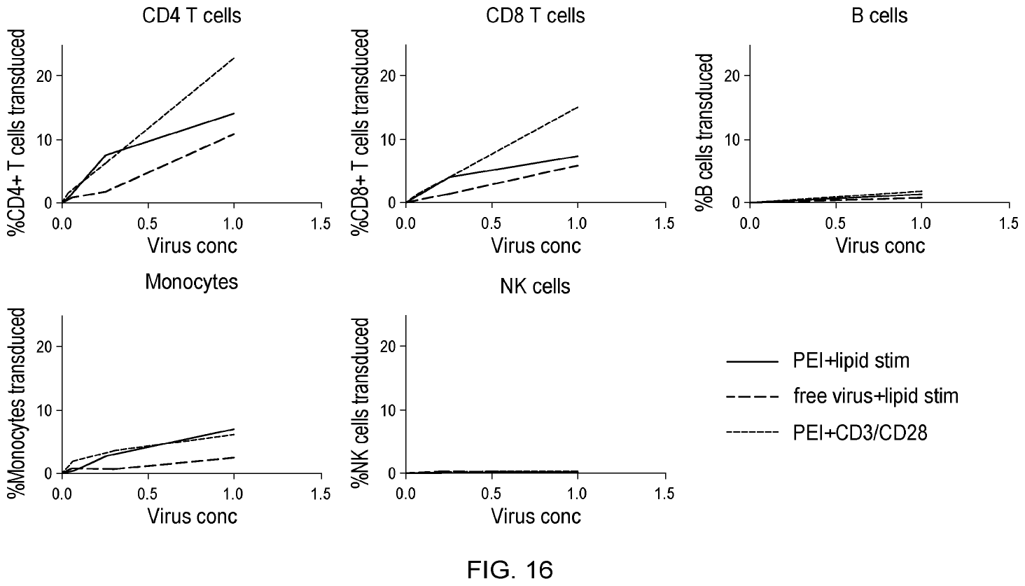

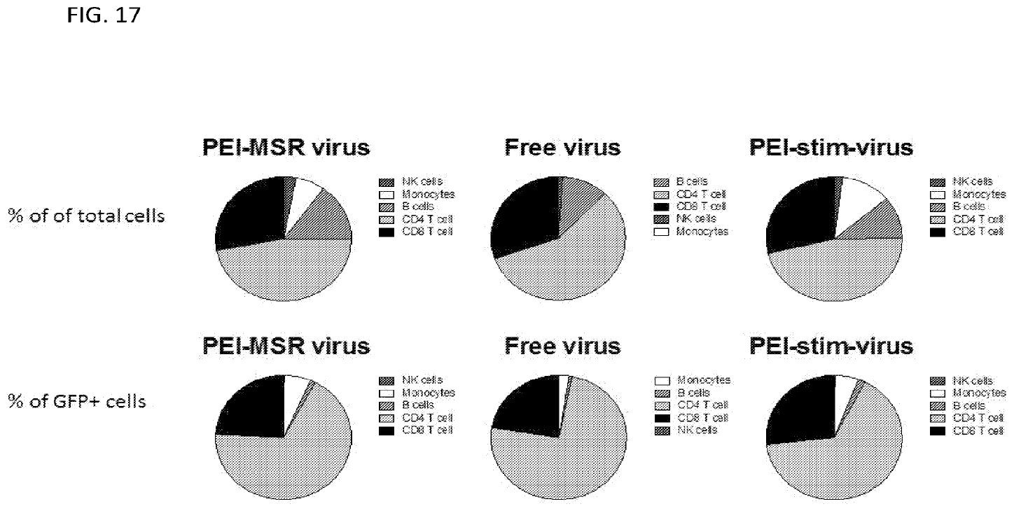

FIG. 15 compares all three conditions, where conditions (1) and (2) are at the highest concentration of stimulatory MSRs. As seen in FIG. 15, condition (3) where stimulatory cues are bound to the PEI-MSRs produces the highest relative transduction efficiency. The same formulations were used to study MSR-mediated transduction with human peripheral blood mononuclear cells (PBMCs). In FIG. 16, the transduction in various cell populations as a function of virus concentration is shown at the highest level of stimulation for conditions (1) and (2). FIG. 17 shows the proportion of each cell population present in the total GFP+ transduced cell fraction, and in the total cell population collected at the highest level of stimulation for conditions (1) and (2).

### Example E. In Vivo Study of MSR Induced T Cell Transduction

A composition of mesoporous silica particle conjugated to viral vectors is injected under the skin of mice. Approximately 5-7 days later, MSRs adsorbed with a virus encoding an anti-mouse CD19 CAR is injected at this site. The depletion of CD19+ B cells in the blood of mice will be monitored as an indication that anti-CD19 CARTs have been generated. The presence of these CARTs is confirmed in the blood and bone marrow. Detailed histological assessment of the injection site as well as draining lymph nodes, the spleen, and liver, using in situ hybridization for the CAR transgene is conducted to assess the leakage of the virus to unwanted sites.

### Example F. Drug Loading onto Mesoporous Silica Microparticles

A variety of drugs may be loaded onto the mesoporous silica microparticles.

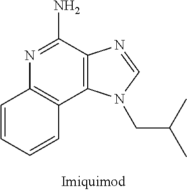 [SMILES: CC(C)Cn1cnc2c(N)nc3ccccc3c21]

### 1. Example 1: Loading of TLR7 Agonist onto Mesoporous Silica Microparticles

A solution of Imiquimod, in chloroform is added to a slurry of 100 mg silica microparticles in 2.0 mL chloroform (a concentration of 100-500 μg of imiquimod per 10 mg of mesoporous silica particles) and shake at 500 rpm at 40 degrees Celsius for 72 hours. The MSPs are centrifuged at 1000 rpm for 3 min and the remaining solution is removed. The MSPs are washed with 2.0 mL of chloroform followed by centrifugation and removal of the supernatant. The wash steps are repeated with ethanol to remove excess and unabsorbed imiquimod. The final microparticles are slurried in water and lyophilized.

### 2. Example 2: In Vitro Drug Release from Mesoporous Silica Particles

10.0 mg (or the equivalent of 300 μg of drug-loaded material) drug-loaded MSPs are suspended in 1.0 mL of pH 7.4 (0.0067M) phosphate buffer and left at 37 degrees Celsius. Samples are collected at 1 h, 3 h, 6 h, 24 h, 2 days, and 5 days; analysis of these samples is performed by UPLC and plotted to a standard analytical curve. Supernatant is removed and replaced with fresh buffer at each timepoint.

The foregoing written specification is considered to be sufficient to enable one skilled in the art to practice the invention. The present invention is not to be limited in scope by the constructs deposited, since the deposited embodiments are intended to illustrate only certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims. Indeed, various modifications of the invention in addition to those shown and described herein will become apparent to those skilled in the art from the foregoing description and fall within the scope of the appended claims.

It is understood that the application of the teachings of the present invention to a specific problem or situation will be within the capabilities of one having ordinary skill in the art in light of the teachings contained herein.

The disclosures of each and every citation in the specification are expressly incorporated herein by reference.

### Example G. In Vivo Study of MSR Induced CAR-T Generation

Mice engrafted with human T cells and B cells (human CD34+ stem cell humanized mice or human peripheral blood mononuclear cell-injected mice) are established using known methods. A composition of mesoporous silica particle conjugated to CAR19 lentivirus is injected under the skin of mice to transduce T cells. The presence of CAR19-expressing T cells (using anti-CAR19 idiotype antibody staining) and the depletion of CD19+ B cells in the blood of mice treated with MSR-CAR19 lentivirus conjugates is monitored using flow cytometry on serial blood collection samples (day 0 pre-injection and twice weekly between day 1 to day 21 following MSR-virus injection) and compared to control mice injected with MSR-GFP lentivirus as an indication that anti-CD19 CARTs have been generated and are functional in killing their target. The concentration of human interferon-gamma and tumor necrosis factor alpha is determined from the same blood samples as a second biomarker for CD19 CAR T cell generation and activation. Detailed histological assessment of the injection site as well as lymph nodes, bone marrow, the spleen, and liver using in situ hybridization for the CAR transgene is conducted to assess the leakage of the virus to unwanted sites and study trafficking of the generated CAR19 T cells to these sites.

In another experiment, human T and B cell-containing mice are intravenously injected with a CD19-expressing Nalm6 leukemia tumor that expresses a luciferase reporter gene. Cohorts of mice are injected under the skin with a single injection of a composition of mesoporous silica particles conjugated to CAR19 or GFP lentivirus from 7 days before to 7 days following tumor injection to transduce T cells. The Nalm6 tumor burden is monitored by luciferase signal on IVIS imaging to study anti-tumor efficacy of the generated anti-CD19 CARTs. The presence of CAR19-expressing T cells and the depletion of CD19+ B cells in the blood of mice treated with MSR-CAR19 lentivirus conjugates is monitored on serial blood collection samples (day 0 pre-injection and twice weekly between day 1 to day 21 following MSR-virus injection) and compared to control mice injected with MSR-GFP lentivirus. The concentration of human interferon-gamma and tumor necrosis factor alpha is determined from the same blood samples as a second biomarker for CD19 CAR T cell generation and activation.

These studies are repeated with MSR-lentivurs conjugates for other cancer/tumor targets, including but not limited to BCMA, CD20, CD22, CD123, EGFRvIII, CLL-1, and combinations thereof (with each other and/or CD19).

1. A composition, comprising a first population of mesoporous silica particles and a viral vector.

2. The composition according to claim 1, wherein the viral vector is conjugated to the first population of mesoporous silica particles.

3. The composition according to claim 2, wherein the viral vector is electrostatically or covalently conjugated to the first population of mesoporous silica particles.

4. The composition according to claim 2, wherein the first population of mesoporous silica particles are surface modified.

5. The composition according to claim 4, wherein the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH2-CH₂—)₁₋₂₅ linker.

6. The composition according to claim 5, wherein the surface modification on the first population of mesoporous silica particles is a primary, secondary, tertiary, or quarternary amine.

7. The composition according to claim 5, wherein the surface modification on the first population of mesoporous silica particles is a polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC).

8. The composition according to any of the preceding claims, wherein the viral vector is a retrovirus, adenovirus, adeno-associated virus, herpes virus, or lentivirus.

9. The composition according to any of the preceding claims, wherein the viral vector comprises an expression vector comprising a recombinant polynucleotide comprising an expression control sequence operatively linked to a nucleotide sequence to be expressed.

10. The composition according to claim 9, wherein the nucleotide sequence encodes a chimeric antigen receptor (CAR), an engineered TCR, one or more cytokines, one or more chemokines, an shRNA to block an inhibitory molecule, or wherein the nucleotide sequence comprises an mRNA to induce expression of a protein.

11. The composition according to claim 10, wherein the nucleotide sequence encodes a polypeptide engineered to target a tumor antigen.

12. The composition according to claim 11, wherein the polypeptide targets a tumor antigen selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof.

13. The composition according to any of claims 10 to 12, wherein the protein is a CAR that comprises an antigen binding domain, a transmembrane domain, a costimulatory signaling region, and a signaling domain.

14. The composition according to claim 13, wherein the signaling domain is a CD3 zeta signaling domain.

15. The composition of any of the preceding claims, further comprising a T cell stimulating compound or tumor antigen.

16. The composition according to claim 15, wherein the T cell stimulating compound or the tumor antigen is conjugated to or adsorbed on the first population of mesoporous silica particles or a second population of mesoporous silica particles, and wherein the T-cell stimulating compound is IL-2, IL-15, anti-CD2 mAb, anti-CD3 mAb, anti-CD28 mAb, neo-antigen peptides from shared antigens such as TRP2, gp100, tumor cell lysate, CD19, CD20, CD22, ROR1, mesothelin, CD33/IL3Ra, c-Met, PSMA, Glycolipid F77, EGFRvIII, GD-2, NY-ESO-1 TCR, MAGE A3 TCR, or combinations thereof.

17. The composition according to claim 16, wherein the T cell stimulating compound or tumor antigen is conjugated to or adsorbed on the first population of mesoporous silica particles.

18. The composition according to claim 16, comprising the second population of mesoporous silica particles, and wherein the T cell stimulating compound or tumor antigen is conjugated to the second population of mesoporous silica particles or to a lipid envelope on the surface of the second population of mesoporous silica particles.

19. The composition of any of claims 15 to 18, further comprising a cytokine.

20. The composition of claim 19, wherein the cytokine is conjugated to or adsorbed on the first or second population of mesoporous silica particles.

21. The composition of any of claim 19 or 20, wherein the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof.

22. The composition of any of the preceding claims, wherein the mesoporous silica particles comprise pores of between 2-50 nm in diameter.

23. The composition of any of the preceding claims, wherein the mesoporous silica particles have a surface area of at least about 100 m²/g.

24. The composition of any of the preceding claims, wherein the composition is suitable for injectable use.

25. A method comprising:
contacting T lymphocytes with a composition comprising a first population of mesoporous silica particles and a viral vector;
wherein the viral vector comprises an expression vector comprising a recombinant polynucleotide comprising an expression control sequence operatively linked to a nucleotide sequence to be expressed.

26. The method according to claim 25, wherein the contacting occurs in vitro.

27. The method according to claim 25, wherein the T lymphocytes are activated before or after contacting with the first population of mesoporous silica particles.

28. The method according to any of claims 25-27, wherein the viral vector is conjugated to the first population of mesoporous silica particles.

29. The method according to claim 28, wherein the viral vector is electrostatically or covalently conjugated to the first population of mesoporous silica particles.

30. The method according to any of claims 25 to 29, wherein the first population of mesoporous silica particles are surface modified.

31. The method according to claim 30, wherein the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH₂—CH₂—)₁₋₂₅ linker.

32. The method according to claim 31, wherein the surface modification on the first population of mesoporous silica particles is a primary, secondary, tertiary, or quarternary amine.

33. The method according to claim 31, wherein the first population of mesoporous silica particles are surface modified with polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC).

34. The method according to any of claims 25 to 33, wherein the viral vector is a lentivirus, retrovirus, or adenovirus.

35. The method according to any of claims 25 to 34, wherein the nucleotide sequence encodes a chimeric antigen receptor (CAR).

36. The method according to claim 35, wherein the CAR is engineered to target a tumor antigen.

37. The method according to any of claims 25 to 36, wherein the T lymphocytes are activated by contacting the T lymphocytes with a T cell stimulating compound or tumor antigen.

38. The method according to claim 37, wherein the T cell stimulating compound or tumor antigen is conjugated to or adsorbed on the first population of mesoporous silica particles or a second population of mesoporous silica particles.

39. The method according to claim 38, wherein the T cell stimulating compound or tumor antigen is conjugated to or adsorbed on the first population of mesoporous silica particles.

40. The method according to claim 39, wherein the T cell stimulating compound or tumor antigen is conjugated directly to the second population of mesoporous silica particles or to a lipid envelope on the surface of the second population of mesoporous silica particles.

41. The method of any of claims 25 to 40, further comprising contacting the T lymphocytes with a cytokine.

42. The method of claim 41, wherein the cytokine is in the medium or conjugated to or adsorbed on the first or second population of mesoporous silica particles.

43. The method of any of claims 40 to 42, wherein the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof.

44. A method of genetically transducing T lymphocytes with a recombinant polynucleotide in vivo, comprising:
administering to a subject, having one or more T lymphocytes, a composition comprising a first population of mesoporous silica particles and a viral vector;
wherein the viral vector comprises an expression vector comprising a recombinant polynucleotide comprising an expression control sequence operatively linked to a nucleotide sequence to be expressed, and
wherein when the composition contacts one or more T lymphocytes, the T lymphocytes are genetically transduced with the recombinant polynucleotide.

45. The method according to claim 44, wherein the viral vector is conjugated to the first population of mesoporous silica particles.

46. The method according to claim 45, wherein the viral vector is electrostatically or covalently conjugated to the first population of mesoporous silica particles.

47. The method according to any of claims 44 to 46, wherein the first population of mesoporous silica particles are surface modified.

48. The method according to claim 47, wherein the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH₂—CH₂—)₁₋₂₅ linker.

49. The method according to claim 48, wherein the surface modification on the first population of mesoporous silica particles is a primary, secondary, tertiary, or quarternary amine.

50. The method according to any of claims 44 to 48, wherein the first population of mesoporous silica particles are surface modified with polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC).

51. The method according to any of claims 44 to 50, wherein the viral vector is a lentivirus, retrovirus, or adenovirus.

52. The method according to any of claims 44 to 51, wherein the nucleotide sequence encodes a chimeric antigen receptor (CAR).

53. The method according to claim 51, wherein the CAR is engineered to target a tumor antigen.

54. The method according to any of claims 44 to 53, wherein the composition further comprises a T cell stimulating compound or tumor antigen conjugated to or adsorbed on the first population of mesoporous silica particles or a second population of mesoporous silica particles.

55. The method according to claim 54, wherein the T cell stimulating compound or tumor antigen is conjugated to or adsorbed on the first population of mesoporous silica particles.

56. The method according to claim 54, wherein the composition comprises the second population of mesoporous silica particles, and wherein the T cell stimulating compound or tumor antigen is conjugated directly to the second population of mesoporous silica particles or to a lipid envelope on the surface of the second population of mesoporous silica particles.

57. The method according to any of claims 44 to 56, wherein the first or second population of mesoporous silica particles further comprises a cytokine conjugated to or adsorbed on the first or second population of mesoporous silica particles.

58. The method according to claim 57, wherein the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof.

59. The method according to any of claims 44 to 58, wherein the subject's T lymphocytes expand in vivo.

60. A method of expanding a T lymphocyte population in vitro, comprising
(a) contacting the T lymphocyte population with a composition comprising a first population of mesoporous silica particles and a viral vector to provide a transduced T lymphocyte population; and
(b) contacting the transduced T lymphocyte population with a T cell stimulating compound or tumor antigen and optionally, a cytokine;
wherein the viral vector comprises an expression vector comprising a recombinant polynucleotide comprising an expression control sequence operatively linked to a nucleotide sequence to be expressed.

61. The method according to claim 60, wherein the viral vector is conjugated to the first population of mesoporous silica particles.

62. The method according to claim 61, wherein the viral vector is electrostatically or covalently conjugated to the first population of mesoporous silica particles.

63. The method according to any of claims 60 to 62, wherein the first population of mesoporous silica particles are surface modified.

64. The method according to claim 63, wherein the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH₂—CH₂—)₁₋₂₅ linker.

65. The method according to claim 64, wherein the surface modification on the first population of mesoporous silica particles is a primary, secondary, tertiary, or quarternary amine.

66. The method according to any of claims 60 to 65, wherein the first population of mesoporous silica particles are surface modified with polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC).

67. The method according to any of claims 60 to 66, wherein the viral vector is a lentivirus, retrovirus, or adenovirus.

68. The method according to any of claims 60 to 67, wherein the nucleotide sequence encodes a chimeric antigen receptor (CAR).

69. The method according to claim 68, wherein the CAR is engineered to target a tumor antigen.

70. The method according to any of claims 60 to 69, wherein the T cell stimulating compound or tumor antigen is conjugated to or adsorbed on the first population of mesoporous silica particles or a second population of mesoporous silica particles, and wherein the T-cell stimulating compound or tumor antigen is IL-2, IL-15, anti-CD2 mAb, anti-CD3 mAb, anti-CD28 mAb, neo-antigen peptides, CD19, CD20, CD22, ROR1, mesothelin, CD33/IL3Ra, c-Met, PSMA, Glycolipid F77, EGFRvIII, GD-2, NY-ESO-1 TCR, MAGE A3 TCR, or combinations thereof.

71. The method according to claim 70, wherein the T cell stimulating compound or tumor antigen is conjugated to or adsorbed on the first population of mesoporous silica particles.

72. The method according to claim 70, comprising the second population of mesoporous silica particles, and wherein the T cell stimulating compound or tumor antigen is conjugated to the second population of mesoporous silica particles or to a lipid envelope on the surface of the second population of mesoporous silica particles.

73. The method of any of claims 60 to 72, further comprising:
(c) contacting the T lymphocytes with a cytokine;
wherein the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof.

74. A method of treating a subject having a disease, disorder, or condition associated with an elevated expression of a tumor antigen, the method comprising:
administering to the subject a composition comprising a first population of mesoporous silica particles and a viral vector,
wherein the viral vector comprises a recombinant polynucleotide comprising an expression control sequence operatively linked to a nucleotide sequence that encodes a chimeric antigen receptor (CAR) that is engineered to target the tumor antigen,
thereby treating the subject.

75. The method according to claim 74, wherein the viral vector is conjugated to the first population of mesoporous silica particles.

76. The method according to claim 75, wherein the viral vector is electrostatically or covalently conjugated to the first population of mesoporous silica particles.

77. The method according to any of claims 74 to 76, wherein the first population of mesoporous silica particles are surface modified.

78. The method according to claim 77, wherein the surface modification on the first population of mesoporous silica particles is —OH (hydroxyl), amine, carboxylic acid, phosphonate, halide, azide, alkyne, epoxide, sulfhydryl, polyethyleneimine, a hydrophobic moiety, or salts thereof, optionally using a C₁ to C₂₀ alkyl or (—O(CH₂—CH₂—)₁₋₂₅ linker.

79. The method according to claim 78, wherein the surface modification on the first population of mesoporous silica particles is a primary, secondary, tertiary, or quarternary amine.

80. The method according to any of claims 74 to 78, wherein the first population of mesoporous silica particles are surface modified with polyethyleneimine having an average molecular weight of about 1000 to 20,000 Da, about 1,200 to 15,000 Da, about 1,500 to 12,000 Da, about 2,000 Da, about 3,000 Da, about 4,000 Da, about 5,000 Da, about 6,000 Da, about 7,000 Da, about 8,000 Da, about 9,000 Da, or about 10,000 Da, as measured by gel permeation chromatography (GPC).

81. The method according to any of claims 74 to 80, wherein the viral vector is a lentivirus, retrovirus, or adenovirus.

82. The method according to any of claims 74 to 81, wherein the composition further comprises a T cell stimulating compound or tumor antigen conjugated to or adsorbed on the first population of mesoporous silica particles or a second population of mesoporous silica particles.

83. The method according to claim 82, wherein the T cell stimulating compound or tumor antigen is conjugated to or adsorbed on the first population of mesoporous silica particles.

84. The method according to claim 83, wherein the composition comprises the second population of mesoporous silica particles, and wherein the T cell stimulating compound or tumor antigen is conjugated directly to the second population of mesoporous silica particles or to a lipid envelope on the surface of the second population of mesoporous silica particles, and wherein the T-cell stimulating compound or tumor antigen is IL-2, IL-15, anti-CD2 mAb, anti-CD3 mAb, anti-CD28 mAb, neo-antigen peptides, CD19, CD20, CD22, ROR1, mesothelin, CD33/IL3Ra, c-Met, PSMA, Glycolipid F77, EGFRvIII, GD-2, NY-ESO-1 TCR, MAGE A3 TCR, or combinations thereof.

85. The method according to any of claims 74 to 84, wherein the first or second population of mesoporous silica particles further comprises a cytokine conjugated to or adsorbed on the first or second population of mesoporous silica particles.

86. The method according to claim 85, wherein the cytokine is IL-1, IL-2, IL-4, IL-5, IL-7, IL-10, IL-12, IL-15, IL-17, IL-21, or transforming growth factor beta (TGF-β), or an agonist thereof, a mimetic thereof, a variant thereof, a functional fragment thereof, or a combination thereof.

87. A method of delivering a viral vector to a desired site of action in a subject, comprising administering to the subject a composition comprising a first population of mesoporous silica particles and the viral vector.

88. The method according to claim 87, wherein the viral vector is conjugated to the first population of mesoporous silica particles.

89. The method according to claim 88, wherein the viral vector is electrostatically or covalently conjugated to the first population of mesoporous silica particles.

90. The method according to any of claims 87 to 89, wherein the first population of mesoporous silica particles are surface modified.

91. The method according to claim 90, wherein the surface modification on the first population of mesoporous silica particles is C₁₋₂₀ alkyl amine, C₁₋₂₀ carboxylic acid, C₁₋₂₀ azide, and substituted or unsubstituted C₁₋₂₀ alkyl.

92. The method according to claim 91, wherein the surface modification on the first population of mesoporous silica particles is a primary, secondary, tertiary, or quarternary amine.

93. The method according to any of claims 87 to 92, wherein the viral vector is a retrovirus, adenovirus, adeno-associated virus, herpes virus, or lentivirus.

94. The method according to any of claims 87 to 93, wherein the first population of mesoporous silica particles comprise pores of between 2-50 nm in diameter.

95. The method of any of claims 87 to 94, wherein the first population of mesoporous silica particles have a surface area of at least about 100 m²/g.

96. A method of expanding a chimeric antigen receptor (CAR) T (CAR-T) cell population, comprising contacting the CAR-T cell population with mesoporous silica particles conjugated to a targeting moiety, wherein the targeting moiety is complementary to the CAR.

97. The method according to claim 96, wherein the CAR is a protein engineered to target a tumor antigen.

98. The method according to any of claim 96 or 97, wherein the tumor antigen is selected from the group consisting of selected from the group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and any combination thereof.

99. The composition according to any of claims 1-24, or the methods according to any of claims 25-98, wherein the mesoporous silica particles are in the form of mesoporous silica rods.

100. A composition comprising mesoporous silica particles conjugated to polyethylenimine.

101. The composition of claim 100, wherein the mesoporous silica particles are in the form of mesoporous silica rods.

102. The composition of claim 100 or 101, further comprising an active agent.

103. The composition of claim 102, wherein the active agent is conjugated to or adsorbed on the mesoporous silica particles.

104. A method of delivering an active agent to a desired site of action in a subject, comprising administering to the subject the composition of claim 102 or 103.

105. The method according to claim 104, wherein the composition provides sustained delivery of the active agent to the subject.

106. A method of treating a subject having a disease, disorder, or condition, the method comprising: administering to the subject the composition of claim 102 or 103.

107. The method of claim 106, wherein the disease, disorder, or condition is associated with a tumor antigen.


SEQUENCE LISTING (692 sequences)

SEQ ID NO: 1 - DNA, 1184 bp, Artificial Sequence
  source: /note=“Description of Artificial Sequence: Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 2 - protein, 21 aa, Artificial Sequence
  source: /note=“Description of Artificial Sequence: Synthetic peptide”
MALPVTALLLPLALLLHAARP

SEQ ID NO: 3 - DNA, 63 bp, Artificial Sequence
  source: /note=“Description of Artificial Sequence: Synthetic oligonucleotide”
atggccctgcctgtgacagccctgctgctgcctctggctctgctgctgcatgccgctagaccc

SEQ ID NO: 4 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 5 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 6 - protein, 230 aa, Artificial Sequence
  source: /note=“Description of Artificial Sequence: Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 7 - DNA, 690 bp, Artificial Sequence
  source: /note=“Description of Artificial Sequence: Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 8 - protein, 282 aa, Artificial Sequence
  source: /note=“Description of Artificial Sequence: Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 9 - DNA, 847 bp, Artificial Sequence
  source: /note=“Description of Artificial Sequence: Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 10 - protein, 10 aa, Artificial Sequence
  source: /note=“Description of Artificial Sequence: Synthetic peptide”
GGGGSGGGGS

SEQ ID NO: 11 - DNA, 30 bp, Artificial Sequence
  source: /note=“Description of Artificial Sequence: Synthetic oligonucleotide”
ggtggcggaggttctggaggtggaggttcc

SEQ ID NO: 12 - protein, 24 aa, Artificial Sequence
  source: /note=“Description of Artificial Sequence: Synthetic peptide”
IYIWAPLAGTCGVLLLSLVITLYC

SEQ ID NO: 13 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 14 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 15 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 17 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 18 - protein, 112 aa, Artificial Sequence
  source: /note=“Description of Artificial Sequence: Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 336 bp, Artificial Sequence
  source: /note=“Description of Artificial Sequence: Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - protein, 112 aa, Artificial Sequence
  source: /note=“Description of Artificial Sequence: Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 336 bp, Artificial Sequence
  source: /note=“Description of Artificial Sequence: Synthetic polynucleotide”
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 5 aa, Artificial Sequence
  source: /note=“Description of Artificial Sequence: Synthetic peptide”
  source: /note=“See specification as filed for detailed description of substitutions and preferred embodiments”
GGGGS

SEQ ID NO: 23 - DNA, 30 bp, Artificial Sequence; identical to SEQ ID NO: 11
  source: /note=“Description of Artificial Sequence: Synthetic oligonucleotide”

SEQ ID NO: 24 - protein, 150 aa, Artificial Sequence [sequence omitted]

SEQ ID NO: 25 - DNA, 450 bp, Artificial Sequence
  source: /note=“Description of Artificial Sequence: Synthetic polynucleotide”
cccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtc

SEQ ID NO: 26 - protein, 394 aa, Artificial Sequence
  source: /note=“Description of Artificial Sequence: Synthetic polypeptide”
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1182 bp, Artificial Sequence
  source: /note=“Description of Artificial Sequence: Synthetic polynucleotide”
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 28 - protein, 40 aa, Artificial Sequence
  source: /note=“Description of Artificial Sequence: Synthetic polypeptide”
  SITE (1)..(40): /note=“This sequence may encompass 1-10 ′Gly Gly Gly Ser′ repeating units”
GGGSGGGSGGGSGGGSGGGSGGGSGGGSGGGSGGGSGGGS

SEQ ID NO: 29 - protein, 20 aa, Artificial Sequence
  source: /note=“Description of Artificial Sequence: Synthetic peptide”
GGGGSGGGGSGGGGSGGGGS

SEQ ID NO: 30 - protein, 15 aa, Artificial Sequence
  source: /note=“Description of Artificial Sequence: Synthetic peptide”
GGGGSGGGGSGGGGS

SEQ ID NO: 31 - protein, 4 aa, Artificial Sequence
  source: /note=“Description of Artificial Sequence: Synthetic peptide”
GGGS

SEQ ID NO: 32-33 - skipped (2 entries)

SEQ ID NO: 34 - RNA, 5000 nt, Artificial Sequence
  source: /note=“Description of Artificial Sequence: Synthetic polynucleotide”
  misc_feature (1)..(5000): /note=“This sequence may encompass 50-5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35-38 - skipped (4 entries)

SEQ ID NO: 39 - protein, 373 aa, Artificial Sequence
  source: /note=“Description of Artificial Sequence: Synthetic polypeptide”
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40-43 - skipped (4 entries)

SEQ ID NO: 44 - protein, 5 aa, Artificial Sequence
  source: /note=“Description of Artificial Sequence: Synthetic peptide”
SYAMS

SEQ ID NO: 45 - protein, 17 aa, Artificial Sequence
  source: /note=“Description of Artificial Sequence: Synthetic peptide”
AISGSGGSTYYADSVKG

SEQ ID NO: 46 - protein, 14 aa, Artificial Sequence
  source: /note=“Description of Artificial Sequence: Synthetic peptide”
REWVPYDVSWYFDY

SEQ ID NO: 47 - protein, 7 aa, Artificial Sequence
  source: /note=“Description of Artificial Sequence: Synthetic peptide”
GFTFSSY

SEQ ID NO: 48 - protein, 6 aa, Artificial Sequence
  source: /note=“Description of Artificial Sequence: Synthetic peptide”
SGSGGS

SEQ ID NO: 49 - protein, 8 aa, Artificial Sequence
  source: /note=“Description of Artificial Sequence: Synthetic peptide”
GFTFSSYA

SEQ ID NO: 50 - protein, 8 aa, Artificial Sequence
  source: /note=“Description of Artificial Sequence: Synthetic peptide”
ISGSGGST

SEQ ID NO: 51 - protein, 16 aa, Artificial Sequence
  source: /note=“Description of Artificial Sequence: Synthetic peptide”
ARREWVPYDVSWYFDY

SEQ ID NO: 52 - protein, 123 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSS

SEQ ID NO: 53 - DNA, 369 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctcc

SEQ ID NO: 54 - protein, 11 aa, Artificial Sequence
  source: /note=“Description of Artificial Sequence: Synthetic peptide”
RASQSISSYLN

SEQ ID NO: 55 - protein, 7 aa, Artificial Sequence
  source: /note=“Description of Artificial Sequence: Synthetic peptide”
AASSLQS

SEQ ID NO: 56 - protein, 9 aa, Artificial Sequence
  source: /note=“Description of Artificial Sequence: Synthetic peptide”
QQSYSTPLT

SEQ ID NO: 57 - protein, 7 aa, Artificial Sequence
  source: /note=“Description of Artificial Sequence: Synthetic peptide”
SQSISSY

SEQ ID NO: 58 - protein, 3 aa, Artificial Sequence
  source: /note=“Description of Artificial Sequence: Synthetic peptide”
AAS

SEQ ID NO: 59 - protein, 6 aa, Artificial Sequence
  source: /note=“Description of Artificial Sequence: Synthetic peptide”
SYSTPL

SEQ ID NO: 60 - protein, 6 aa, Artificial Sequence
  source: /note=“Description of Artificial Sequence: Synthetic peptide”
QSISSY

SEQ ID NO: 61 - protein, 107 aa, Artificial Sequence [sequence omitted]

SEQ ID NO: 62 - DNA, 321 bp, Artificial Sequence
  source: /note=“Description of Artificial Sequence: Synthetic polynucleotide”
gacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 63 - protein, 20 aa, Artificial Sequence
  source: /note=“Description of Artificial Sequence: Synthetic peptide”
GGGGSGGGGSGGGGSGGGGS

SEQ ID NO: 64 - protein, 250 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 65 - DNA, 750 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 66 - protein, 473 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 1419 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 68 - protein, 12 aa, Artificial Sequence
  source: /note=“Description of Artificial Sequence: Synthetic peptide”
REWWYDDWYLDY

SEQ ID NO: 69 - protein, 14 aa, Artificial Sequence
  source: /note=“Description of Artificial Sequence: Synthetic peptide”
ARREWWYDDWYLDY

SEQ ID NO: 70 - protein, 121 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSS

SEQ ID NO: 71 - DNA, 363 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctcc

SEQ ID NO: 72 - protein, 248 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 73 - DNA, 744 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 74 - protein, 471 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 1413 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 76 - protein, 12 aa, Artificial Sequence
  source: /note=“Description of Artificial Sequence: Synthetic peptide”
REWWGESWLFDY

SEQ ID NO: 77 - protein, 14 aa, Artificial Sequence
  source: /note=“Description of Artificial Sequence: Synthetic peptide”
ARREWWGESWLFDY

SEQ ID NO: 78 - protein, 121 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSS

SEQ ID NO: 79 - DNA, 363 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctcc

SEQ ID NO: 80 - protein, 248 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 81 - DNA, 744 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 82 - protein, 471 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - DNA, 1413 bp, Artificial Sequence
  source: /note=“Description of Artificial Sequence: Synthetic polynucleotide”
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 84 - protein, 14 aa, Artificial Sequence
  source: /note=“Description of Artificial Sequence: Synthetic peptide”
  VARIANT (4)..(4): /replace=“ ”
  VARIANT (5)..(5): /replace=“ ”
  VARIANT (6)..(6): /replace=“Tyr”
  VARIANT (7)..(7): /replace=“Tyr” or “Asp”
  VARIANT (8)..(8): /replace=“Asp” or “Val”
  VARIANT (9)..(9): /replace=“Asp”
  VARIANT (11)..(11): /replace=“Tyr”
  VARIANT (12)..(12): /replace=“Leu”
  SITE (1)..(14): /note=“Variant residues given in the sequence have no preference with respect to those in the annotations for variant positions”
REWVPWGESWLFDY

SEQ ID NO: 85 - protein, 16 aa, Artificial Sequence
  source: /note=“Description of Artificial Sequence: Synthetic peptide”
  VARIANT (6)..(6): /replace=“ ”
  VARIANT (7)..(7): /replace=“ ”
  VARIANT (8)..(8): /replace=“Tyr”
  VARIANT (9)..(9): /replace=“Tyr” or “Asp”
  VARIANT (10)..(10): /replace=“Asp” or “Val”
  VARIANT (11)..(11): /replace=“Asp”
  VARIANT (13)..(13): /replace=“Tyr”
  VARIANT (14)..(14): /replace=“Leu”
  SITE (1)..(16): /note=“Variant residues given in the sequence have no preference with respect to those in the annotations for variant positions”
ARREWVPWGESWLFDY

SEQ ID NO: 86 - protein, 5 aa, Artificial Sequence
  source: /note=“Description of Artificial Sequence: Synthetic peptide”
SYGMH

SEQ ID NO: 87 - protein, 17 aa, Artificial Sequence
  source: /note=“Description of Artificial Sequence: Synthetic peptide”
VISYDGSNKYYADSVKG

SEQ ID NO: 88 - protein, 14 aa, Artificial Sequence
  source: /note=“Description of Artificial Sequence: Synthetic peptide”
SGYALHDDYYGLDV

SEQ ID NO: 89 - protein, 6 aa, Artificial Sequence
  source: /note=“Description of Artificial Sequence: Synthetic peptide”
SYDGSN

SEQ ID NO: 90 - protein, 8 aa, Artificial Sequence
  source: /note=“Description of Artificial Sequence: Synthetic peptide”
GFTFSSYG

SEQ ID NO: 91 - protein, 8 aa, Artificial Sequence
  source: /note=“Description of Artificial Sequence: Synthetic peptide”
ISYDGSNK

SEQ ID NO: 92 - protein, 16 aa, Artificial Sequence
  source: /note=“Description of Artificial Sequence: Synthetic peptide”
GGSGYALHDDYYGLDV

SEQ ID NO: 93 - protein, 123 aa, Artificial Sequence
  source: /note=“Description of Artificial Sequence: Synthetic polypeptide”
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSS

SEQ ID NO: 94 - DNA, 369 bp, Artificial Sequence
  source: /note=“Description of Artificial Sequence: Synthetic polynucleotide”
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagc

SEQ ID NO: 95 - protein, 14 aa, Artificial Sequence
  source: /note=“Description of Artificial Sequence: Synthetic peptide”
TGTSSDVGGYNYVS

SEQ ID NO: 96 - protein, 7 aa, Artificial Sequence
  source: /note=“Description of Artificial Sequence: Synthetic peptide”
DVSNRPS

SEQ ID NO: 97 - protein, 11 aa, Artificial Sequence
  source: /note=“Description of Artificial Sequence: Synthetic peptide”
SSYTSSSTLYV

SEQ ID NO: 98 - protein, 10 aa, Artificial Sequence
  source: /note=“Description of Artificial Sequence: Synthetic peptide”
TSSDVGGYNY

SEQ ID NO: 99 - protein, 3 aa, Artificial Sequence
  source: /note=“Description of Artificial Sequence: Synthetic peptide”
DVS

SEQ ID NO: 100 - protein, 8 aa, Artificial Sequence
  source: /note=“Description of Artificial Sequence: Synthetic peptide”
YTSSSTLY

SEQ ID NO: 101 - protein, 9 aa, Artificial Sequence
  source: /note=“Description of Artificial Sequence: Synthetic peptide”
SSDVGGYNY

SEQ ID NO: 102 - protein, 111 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVL

SEQ ID NO: 103 - DNA, 333 bp, Artificial Sequence
  source: /note=“Description of Artificial Sequence: Synthetic polynucleotide”
cagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctg

SEQ ID NO: 104 - protein, 15 aa, Artificial Sequence
  source: /note=“Description of Artificial Sequence: Synthetic peptide”
GGGGSGGGGSGGGGS

SEQ ID NO: 105 - protein, 249 aa, Artificial Sequence
  source: /note=“Description of Artificial Sequence: Synthetic polypeptide”
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVL

SEQ ID NO: 106 - DNA, 747 bp, Artificial Sequence
  source: /note=“Description of Artificial Sequence: Synthetic polynucleotide”
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagcggtggaggaggttcgggcggaggaggatcaggagggggtggatcgcagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctg

SEQ ID NO: 107 - protein, 472 aa, Artificial Sequence
  source: /note=“Description of Artificial Sequence: Synthetic polypeptide”
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - DNA, 1416 bp, Artificial Sequence
  source: /note=“Description of Artificial Sequence: Synthetic polynucleotide”
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagcggtggaggaggttcgggcggaggaggatcaggagggggtggatcgcagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 109 - protein, 17 aa, Artificial Sequence
  source: /note=“Description of Artificial Sequence: Synthetic peptide”
VISYKGSNKYYADSVKG

SEQ ID NO: 110 - protein, 6 aa, Artificial Sequence
  source: /note=“Description of Artificial Sequence: Synthetic peptide”
SYKGSN

SEQ ID NO: 111 - protein, 8 aa, Artificial Sequence
  source: /note=“Description of Artificial Sequence: Synthetic peptide”
ISYKGSNK

SEQ ID NO: 112 - protein, 123 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSS

SEQ ID NO: 113 - DNA, 369 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctct

SEQ ID NO: 114 - protein, 7 aa, Artificial Sequence
  source: /note=“Description of Artificial Sequence: Synthetic peptide”
EVSNRLR

SEQ ID NO: 115 - protein, 11 aa, Artificial Sequence
  source: /note=“Description of Artificial Sequence: Synthetic peptide”
SSYTSSSALYV

SEQ ID NO: 116 - protein, 3 aa, Artificial Sequence
  source: /note=“Description of Artificial Sequence: Synthetic peptide”
EVS

SEQ ID NO: 117 - protein, 8 aa, Artificial Sequence
  source: /note=“Description of Artificial Sequence: Synthetic peptide”
YTSSSALY

SEQ ID NO: 118 - protein, 111 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVL

SEQ ID NO: 119 - DNA, 333 bp, Artificial Sequence
  source: /note=“Description of Artificial Sequence: Synthetic polynucleotide”
cagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctg

SEQ ID NO: 120 - protein, 254 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVL

SEQ ID NO: 121 - DNA, 762 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctg

SEQ ID NO: 122 - protein, 477 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1431 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 124 - protein, 111 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVL

SEQ ID NO: 125 - DNA, 333 bp, Artificial Sequence
  source: /note=“Description of Artificial Sequence: Synthetic polynucleotide”
cagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctg

SEQ ID NO: 126 - protein, 254 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVL

SEQ ID NO: 127 - DNA, 762 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctg

SEQ ID NO: 128 - protein, 477 aa, Artificial Sequence
  source: /note=“Description of Artificial Sequence: Synthetic polypeptide”
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 1431 bp, Artificial Sequence
  source: /note=“Description of Artificial Sequence: Synthetic polynucleotide”
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 130 - protein, 17 aa, Artificial Sequence
  source: /note=“Description of Artificial Sequence: Synthetic peptide”
  VARIANT (5)..(5): /replace=“Lys”
  SITE (1)..(17): /note=“Variant residues given in the sequence have no preference with respect to those in the annotations for variant positions”
VISYDGSNKYYADSVKG

SEQ ID NO: 131 - protein, 7 aa, Artificial Sequence
  source: /note=“Description of Artificial Sequence: Synthetic peptide”
  VARIANT (1)..(1): /replace=“Glu”
  VARIANT (6)..(6): /replace=“Leu”
  VARIANT (7)..(7): /replace=“Arg”
  SITE (1)..(7): /note=“Variant residues given in the sequence have no preference with respect to those in the annotations for variant positions”
DVSNRPS

SEQ ID NO: 132 - protein, 11 aa, Artificial Sequence
  source: /note=“Description of Artificial Sequence: Synthetic peptide”
  VARIANT (8)..(8): /replace=“Ala”
  SITE (1)..(11): /note=“Variant residues given in the sequence have no preference with respect to those in the annotations for variant positions”
SSYTSSSTLYV

SEQ ID NO: 133 - protein, 6 aa, Artificial Sequence
  source: /note=“Description of Artificial Sequence: Synthetic peptide”
  VARIANT (3)..(3): /replace=“Lys”
  SITE (1)..(6): /note=“Variant residues given in the sequence have no preference with respect to those in the annotations for variant positions”
SYDGSN

SEQ ID NO: 134 - protein, 3 aa, Artificial Sequence
  source: /note=“Description of Artificial Sequence: Synthetic peptide”
  VARIANT (1)..(1): /replace=“Glu”
  SITE (1)..(3): /note=“Variant residues given in the sequence have no preference with respect to those in the annotations for variant positions”
DVS

SEQ ID NO: 135 - protein, 8 aa, Artificial Sequence
  source: /note=“Description of Artificial Sequence: Synthetic peptide”
  VARIANT (6)..(6): /replace=“Ala”
  SITE (1)..(8): /note=“Variant residues given in the sequence have no preference with respect to those in the annotations for variant positions”
YTSSSTLY

SEQ ID NO: 136 - protein, 8 aa, Artificial Sequence
  source: /note=“Description of Artificial Sequence: Synthetic peptide”
  VARIANT (4)..(4): /replace=“Lys”
  SITE (1)..(8): /note=“Variant residues given in the sequence have no preference with respect to those in the annotations for variant positions”
ISYDGSNK

SEQ ID NO: 137 - protein, 5 aa, Artificial Sequence
  source: /note=“Description of Artificial Sequence: Synthetic peptide”
GFWMS

SEQ ID NO: 138 - protein, 17 aa, Artificial Sequence
  source: /note=“Description of Artificial Sequence: Synthetic peptide”
NIKQDGSEKYYVDSVRG

SEQ ID NO: 139 - protein, 9 aa, Artificial Sequence
  source: /note=“Description of Artificial Sequence: Synthetic peptide”
ALDYYGMDV

SEQ ID NO: 140 - protein, 7 aa, Artificial Sequence
  source: /note=“Description of Artificial Sequence: Synthetic peptide”
GFTFSGF

SEQ ID NO: 141 - protein, 6 aa, Artificial Sequence
  source: /note=“Description of Artificial Sequence: Synthetic peptide”
KQDGSE

SEQ ID NO: 142 - protein, 8 aa, Artificial Sequence
  source: /note=“Description of Artificial Sequence: Synthetic peptide”
GFTFSGFW

SEQ ID NO: 143 - protein, 8 aa, Artificial Sequence
  source: /note=“Description of Artificial Sequence: Synthetic peptide”
IKQDGSEK

SEQ ID NO: 144 - protein, 11 aa, Artificial Sequence
  source: /note=“Description of Artificial Sequence: Synthetic peptide”
ARALDYYGMDV

SEQ ID NO: 145 - protein, 118 aa, Artificial Sequence
  source: /note=“Description of Artificial Sequence: Synthetic polypeptide”
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSS

SEQ ID NO: 146 - DNA, 354 bp, Artificial Sequence
  source: /note=“Description of Artificial Sequence: Synthetic polynucleotide”
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagc

SEQ ID NO: 147 - protein, 17 aa, Artificial Sequence
  source: /note=“Description of Artificial Sequence: Synthetic peptide”
RSSQSLLDSDDGNTYLD

SEQ ID NO: 148 - protein, 7 aa, Artificial Sequence
  source: /note=“Description of Artificial Sequence: Synthetic peptide”
TLSYRAS

SEQ ID NO: 149 - protein, 10 aa, Artificial Sequence
  source: /note=“Description of Artificial Sequence: Synthetic peptide”
TQRLEFPSIT

SEQ ID NO: 150 - protein, 13 aa, Artificial Sequence
  source: /note=“Description of Artificial Sequence: Synthetic peptide”
SQSLLDSDDGNTY

SEQ ID NO: 151 - protein, 3 aa, Artificial Sequence
  source: /note=“Description of Artificial Sequence: Synthetic peptide”
TLS

SEQ ID NO: 152 - protein, 7 aa, Artificial Sequence
  source: /note=“Description of Artificial Sequence: Synthetic peptide”
RLEFPSI

SEQ ID NO: 153 - protein, 12 aa, Artificial Sequence
  source: /note=“Description of Artificial Sequence: Synthetic peptide”
QSLLDSDDGNTY

SEQ ID NO: 154 - protein, 114 aa, Artificial Sequence
  source: /note=“Description of Artificial Sequence: Synthetic polypeptide”
DIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIK

SEQ ID NO: 155 - DNA, 342 bp, Artificial Sequence
  source: /note=“Description of Artificial Sequence: Synthetic polynucleotide”
gatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaag

SEQ ID NO: 156 - protein, 252 aa, Artificial Sequence
  source: /note=“Description of Artificial Sequence: Synthetic polypeptide”
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSSGGGGSGGGGSGGGGSGGGGSDIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIK

SEQ ID NO: 157 - DNA, 756 bp, Artificial Sequence
  source: /note=“Description of Artificial Sequence: Synthetic polynucleotide”
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaag

SEQ ID NO: 158 - protein, 475 aa, Artificial Sequence
  source: /note=“Description of Artificial Sequence: Synthetic polypeptide”
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSSGGGGSGGGGSGGGGSGGGGSDIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - DNA, 1425 bp, Artificial Sequence
  source: /note=“Description of Artificial Sequence: Synthetic polynucleotide”
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 160 - protein, 5 aa, Artificial Sequence
  source: /note=“Description of Artificial Sequence: Synthetic peptide”
SFRMN

SEQ ID NO: 161 - protein, 17 aa, Artificial Sequence
  source: /note=“Description of Artificial Sequence: Synthetic peptide”
SISSSSSYIYYADSVKG

SEQ ID NO: 162 - protein, 9 aa, Artificial Sequence
  source: /note=“Description of Artificial Sequence: Synthetic peptide”
WLSYYGMDV

SEQ ID NO: 163 - protein, 7 aa, Artificial Sequence
  source: /note=“Description of Artificial Sequence: Synthetic peptide”
GFTFSSF

SEQ ID NO: 164 - protein, 6 aa, Artificial Sequence
  source: /note=“Description of Artificial Sequence: Synthetic peptide”
SSSSSY

SEQ ID NO: 165 - protein, 8 aa, Artificial Sequence
  source: /note=“Description of Artificial Sequence: Synthetic peptide”
GFTFSSFR

SEQ ID NO: 166 - protein, 8 aa, Artificial Sequence
  source: /note=“Description of Artificial Sequence: Synthetic peptide”
ISSSSSYI

SEQ ID NO: 167 - protein, 11 aa, Artificial Sequence
  source: /note=“Description of Artificial Sequence: Synthetic peptide”
ARWLSYYGMDV

SEQ ID NO: 168 - protein, 118 aa, Artificial Sequence
  source: /note=“Description of Artificial Sequence: Synthetic polypeptide”
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSS

SEQ ID NO: 169 - DNA, 354 bp, Artificial Sequence
  source: /note=“Description of Artificial Sequence: Synthetic polynucleotide”
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagc

SEQ ID NO: 170 - protein, 7 aa, Artificial Sequence
  source: /note=“Description of Artificial Sequence: Synthetic peptide”
TLSFRAS

SEQ ID NO: 171 - protein, 9 aa, Artificial Sequence
  source: /note=“Description of Artificial Sequence: Synthetic peptide”
MQRIGFPIT

SEQ ID NO: 172 - protein, 6 aa, Artificial Sequence
  source: /note=“Description of Artificial Sequence: Synthetic peptide”
RIGFPI

SEQ ID NO: 173 - protein, 113 aa, Artificial Sequence
  source: /note=“Description of Artificial Sequence: Synthetic polypeptide”
DIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIK

SEQ ID NO: 174 - DNA, 339 bp, Artificial Sequence
  source: /note=“Description of Artificial Sequence: Synthetic polynucleotide”
gatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaag

SEQ ID NO: 175 - protein, 251 aa, Artificial Sequence
  source: /note=“Description of Artificial Sequence: Synthetic polypeptide”
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSSGGGGSGGGGSGGGGSGGGGSDIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIK

SEQ ID NO: 176 - DNA, 753 bp, Artificial Sequence
  source: /note=“Description of Artificial Sequence: Synthetic polynucleotide”
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaag

SEQ ID NO: 177 - protein, 474 aa, Artificial Sequence
  source: /note=“Description of Artificial Sequence: Synthetic polypeptide”
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSSGGGGSGGGGSGGGGSGGGGSDIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - DNA, 1422 bp, Artificial Sequence
  source: /note=“Description of Artificial Sequence: Synthetic polynucleotide”
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 179 - protein, 5 aa, Artificial Sequence
  source: /note=“Description of Artificial Sequence: Synthetic peptide”
  VARIANT (1)..(1): /replace=“Ser”
  VARIANT (3)..(3): /replace=“Arg”
  VARIANT (5)..(5): /replace=“Asn”
  SITE (1)..(5): /note=“Variant residues given in the sequence have no preference with respect to those in the annotations for variant positions”
GFWMS

SEQ ID NO: 180 - protein, 17 aa, Artificial Sequence
  source: /note=“Description of Artificial Sequence: Synthetic peptide”
  VARIANT (1)..(1): /replace=“Ser”
  VARIANT (3)..(3): /replace=“Ser”
  VARIANT (4)..(4): /replace=“Ser”
  VARIANT (5)..(5): /replace=“Ser”
  VARIANT (6)..(6): /replace=“Ser”
  VARIANT (8)..(8): /replace=“Tyr”
  VARIANT (9)..(9): /replace=“Ile”
  VARIANT (12)..(12): /replace=“Ala”
  VARIANT (16)..(16): /replace=“Lys”
  SITE (1)..(17): /note=“Variant residues given in the sequence have no preference with respect to those in the annotations for variant positions”
NIKQDGSEKYYVDSVRG

SEQ ID NO: 181 - protein, 9 aa, Artificial Sequence
  source: /note=“Description of Artificial Sequence: Synthetic peptide”
  VARIANT (1)..(1): /replace=“Trp”
  VARIANT (3)..(3): /replace=“Ser”
  SITE (1)..(9): /note=“Variant residues given in the sequence have no preference with respect to those in the annotations for variant positions”
ALDYYGMDV

SEQ ID NO: 182 - protein, 7 aa, Artificial Sequence
  source: /note=“Description of Artificial Sequence: Synthetic peptide”
  VARIANT (4)..(4): /replace=“Phe”
  SITE (1)..(7): /note=“Variant residues given in the sequence have no preference with respect to those in the annotations for variant positions”
TLSYRAS

SEQ ID NO: 183 - protein, 10 aa, Artificial Sequence
  source: /note=“Description of Artificial Sequence: Synthetic peptide”
  VARIANT (1)..(1): /replace=“Met”
  VARIANT (4)..(4): /replace=“Ile”
  VARIANT (5)..(5): /replace=“Gly”
  VARIANT (8)..(8): /replace=“ ”
  SITE (1)..(10): /note=“Variant residues given in the sequence have no preference with respect to those in the annotations for variant positions”
TQRLEFPSIT

SEQ ID NO: 184 - protein, 7 aa, Artificial Sequence
  source: /note=“Description of Artificial Sequence: Synthetic peptide”
  VARIANT (6)..(6): /replace=“Ser”
  SITE (1)..(7): /note=“Variant residues given in the sequence have no preference with respect to those in the annotations for variant positions”
GFTFSGF

SEQ ID NO: 185 - protein, 6 aa, Artificial Sequence
  source: /note=“Description of Artificial Sequence: Synthetic peptide”
  VARIANT (1)..(1): /replace=“Ser”
  VARIANT (2)..(2): /replace=“Ser”
  VARIANT (3)..(3): /replace=“Ser”
  VARIANT (4)..(4): /replace=“Ser”
  VARIANT (6)..(6): /replace=“Tyr”
  SITE (1)..(6): /note=“Variant residues given in the sequence have no preference with respect to those in the annotations for variant positions”
KQDGSE

SEQ ID NO: 186 - protein, 7 aa, Artificial Sequence
  source: /note=“Description of Artificial Sequence: Synthetic peptide”
  VARIANT (2)..(2): /replace=“Ile”
  VARIANT (3)..(3): /replace=“Gly”
  VARIANT (6)..(6): /replace=“ ”
  SITE (1)..(7): /note=“Variant residues given in the sequence have no preference with respect to those in the annotations for variant positions”
RLEFPSI

SEQ ID NO: 187 - protein, 8 aa, Artificial Sequence
  source: /note=“Description of Artificial Sequence: Synthetic peptide”
  VARIANT (6)..(6): /replace=“Ser”
  VARIANT (8)..(8): /replace=“Arg”
  SITE (1)..(8): /note=“Variant residues given in the sequence have no preference with respect to those in the annotations for variant positions”
GFTFSGFW

SEQ ID NO: 188 - protein, 8 aa, Artificial Sequence
  source: /note=“Description of Artificial Sequence: Synthetic peptide”
  VARIANT (2)..(2): /replace=“Ser”
  VARIANT (3)..(3): /replace=“Ser”
  VARIANT (4)..(4): /replace=“Ser”
  VARIANT (5)..(5): /replace=“Ser”
  VARIANT (7)..(7): /replace=“Tyr”
  VARIANT (8)..(8): /replace=“Ile”
  SITE (1)..(8): /note=“Variant residues given in the sequence have no preference with respect to those in the annotations for variant positions”
IKQDGSEK

SEQ ID NO: 189 - protein, 11 aa, Artificial Sequence
  source: /note=“Description of Artificial Sequence: Synthetic peptide”
  VARIANT (3)..(3): /replace=“Trp”
  VARIANT (5)..(5): /replace=“Ser”
  SITE (1)..(11): /note=“Variant residues given in the sequence have no preference with respect to those in the annotations for variant positions”
ARALDYYGMDV

SEQ ID NO: 190-201 - skipped (12 entries)

SEQ ID NO: 202 - protein, 69 aa, Artificial Sequence
  source: /note=“Description of Artificial Sequence: Synthetic polypeptide”
TTTPAPRPPTPAPTIASQPLSLRPEACRPAAGGAVHTRGLDFACDIYIWAPLAGTCGVLLLSLVITLYC

SEQ ID NO: 203-251 - skipped (49 entries)

SEQ ID NO: 252 - DNA, 63 bp, Artificial Sequence
  source: /note=“Description of Artificial Sequence: Synthetic oligonucleotide”
atggccctccctgtcaccgctctgttgctgccgcttgctctgctgctccacgcagcgcgaccg

SEQ ID NO: 253 - DNA, 747 bp, Artificial Sequence
  source: /note=“Description of Artificial Sequence: Synthetic polynucleotide”
caggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactc

SEQ ID NO: 254 - DNA, 207 bp, Artificial Sequence
  source: /note=“Description of Artificial Sequence: Synthetic polynucleotide”
acaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgt

SEQ ID NO: 255 - DNA, 126 bp, Artificial Sequence
  source: /note=“Description of Artificial Sequence: Synthetic polynucleotide”
aaacgcgggcgaaaaaaattgctgtatatttttaagcagccatttatgaggcccgttcagacgacgcaggaggaggacggttgctcttgcaggttcccagaagaggaagaagggggctgtgaattg

SEQ ID NO: 256 - DNA, 336 bp, Artificial Sequence
  source: /note=“Description of Artificial Sequence: Synthetic polynucleotide”
cgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 257 - protein, 493 aa, Artificial Sequence
  source: /note=“Description of Artificial Sequence: Synthetic polypeptide”
MALPVTALLLPLALLLHAARPQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 258 - DNA, 1479 bp, Artificial Sequence
  source: /note=“Description of Artificial Sequence: Synthetic polynucleotide”
atggccctccctgtcaccgctctgttgctgccgcttgctctgctgctccacgcagcgcgaccgcaggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactcacaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgtaaacgcgggcgaaaaaaattgctgtatatttttaagcagccatttatgaggcccgttcagacgacgcaggaggaggacggttgctcttgcaggttcccagaagaggaagaagggggctgtgaattgcgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 259-497 - skipped (239 entries)

SEQ ID NO: 498 - protein, 12 aa, Artificial Sequence
  source: /note=“Description of Artificial Sequence: Synthetic peptide”
GDSMLSNSDTWN

SEQ ID NO: 499 - protein, 7 aa, Artificial Sequence
  source: /note=“Description of Artificial Sequence: Synthetic peptide”
SNSDTWN

SEQ ID NO: 500-508 - skipped (9 entries)

SEQ ID NO: 509 - protein, 18 aa, Artificial Sequence
  source: /note=“Description of Artificial Sequence: Synthetic peptide”
RTYHRSTWYDDYASSVRG

SEQ ID NO: 510 - protein, 18 aa, Artificial Sequence
  source: /note=“Description of Artificial Sequence: Synthetic peptide”
RTYHRSTWYDDYASSVRG

SEQ ID NO: 511-519 - skipped (9 entries)

SEQ ID NO: 520 - protein, 15 aa, Artificial Sequence
  source: /note=“Description of Artificial Sequence: Synthetic peptide”
VRLQDGNSWSDAFDV

SEQ ID NO: 521 - protein, 15 aa, Artificial Sequence
  source: /note=“Description of Artificial Sequence: Synthetic peptide”
VRLQDGNSWSDAFDV

SEQ ID NO: 522-647 - skipped (126 entries)

SEQ ID NO: 648 - protein, 14 aa, Artificial Sequence
  source: /note=“Description of Artificial Sequence: Synthetic peptide”
TGTSSDVGGYNYVS

SEQ ID NO: 649-658 - skipped (10 entries)

SEQ ID NO: 659 - protein, 7 aa, Artificial Sequence
  source: /note=“Description of Artificial Sequence: Synthetic peptide”
DVSNRPS

SEQ ID NO: 660-669 - skipped (10 entries)

SEQ ID NO: 670 - protein, 11 aa, Artificial Sequence
  source: /note=“Description of Artificial Sequence: Synthetic peptide”
SSYTSSSTLYV

SEQ ID NO: 671 - protein, 253 aa, Artificial Sequence
  source: /note=“Description of Artificial Sequence: Synthetic polypeptide”
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 672 - protein, 127 aa, Artificial Sequence
  source: /note=“Description of Artificial Sequence: Synthetic polypeptide”
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSS

SEQ ID NO: 673 - protein, 111 aa, Artificial Sequence
  source: /note=“Description of Artificial Sequence: Synthetic polypeptide”
QSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 674 - protein, 13 aa, Artificial Sequence
  source: /note=“Description of Artificial Sequence: Synthetic peptide”
LEEKKGNYVVTDH

SEQ ID NO: 675 - protein, 465 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 676 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 677 - protein, 465 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 678 - protein, 5 aa, Artificial Sequence
  source: /note=“Description of Artificial Sequence: Synthetic peptide”
DYGVS

SEQ ID NO: 679 - protein, 16 aa, Artificial Sequence
  source: /note=“Description of Artificial Sequence: Synthetic peptide”
VIWGSETTYYNSALKS

SEQ ID NO: 680 - protein, 12 aa, Artificial Sequence
  source: /note=“Description of Artificial Sequence: Synthetic peptide”
HYYYGGSYAMDY

SEQ ID NO: 681 - protein, 11 aa, Artificial Sequence
  source: /note=“Description of Artificial Sequence: Synthetic peptide”
RASQDISKYLN

SEQ ID NO: 682 - protein, 7 aa, Artificial Sequence
  source: /note=“Description of Artificial Sequence: Synthetic peptide”
HTSRLHS

SEQ ID NO: 683 - protein, 9 aa, Artificial Sequence
  source: /note=“Description of Artificial Sequence: Synthetic peptide”
QQGNTLPYT

SEQ ID NO: 684 - protein, 486 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 685 - DNA, 1458 bp, Artificial Sequence
  source: /note=“Description of Artificial Sequence: Synthetic polynucleotide”
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 686 - protein, 242 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 687 - protein, 16 aa, Artificial Sequence
  source: /note=“Description of Artificial Sequence: Synthetic peptide”
VIWGSETTYYQSSLKS

SEQ ID NO: 688 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 689 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 690 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 691 - protein, 30 aa, Artificial Sequence
  source: /note=“Description of Artificial Sequence: Synthetic polypeptide”
  SITE (1)..(30): /note=“This sequence may encompass 1-6 ′Gly Gly Gly Gly Ser′ repeating units”
  source: /note=“See specification as filed for detailed description of substitutions and preferred embodiments”
GGGGSGGGGSGGGGSGGGGSGGGGSGGGGS

SEQ ID NO: 692 - protein, 4 aa, Artificial Sequence
  source: /note=“Description of Artificial Sequence: Synthetic peptide”
RGDS